(19) United States
(12) Patent Application Publication
(10) Pub. No.: US 2003/0118991 A1
(43) Pub. Date: Jun. 26, 2003

(54) Ataxia-telangiectasia gene and its genomic organization

(76) Inventor: Yosef Shiloh, Tel Aviv (IL)

Correspondence Address: Kohn & Associates, Suite 410, 30500 Northwestern Hwy., Farmington Hills, MI, 48334, US

(21) Appl. No.: 09/825,476

(22) Filed: Apr. 3, 2001

Related U.S. Application Data
(62) Division of application No. 08/952,014, filed on Feb. 2, 1998, now Pat. No. 6,265,158.
Application No. 08/952,014 is a 371 of international application No. PCT/US96/07025, filed on May 16, 1996.

(51) Int. Cl. C12Q 1/68; C07H 21/04

(52) U.S. Cl. 435/6; 536/23.2

(57) ABSTRACT

A gene, designated ATM, mutations of which cause ataxia-telangiectasia (A-T), that has been purified, isolated, and sequenced as well as mutations of the gene and the genomic organization of the gene. A method for identifying carriers of the defective A-T gene in a population and effective A-T gene products. The detection of A-T mutation carriers for use in determining cancer predisposition and radiation sensitivity so that carrier exposure to radiation can be properly monitored and avoided.

### TECHNICAL FIELD

The present invention relates to the determination of the gene sequence, mutations of which cause ataxia-telangiectasia (A-T), designated ATM, and the use of the gene and gene products in detection of carriers of the A-T gene, and preparing native and transgenic organisms in which the gene products encoded by the ATM gene or its homolog in other species are artificially produced, or the expression of the native ATM gene is modified.

### BACKGROUND OF THE INVENTION

Ataxia-telangiectasia (A-T) is a progressive genetic disorder affecting the central nervous and immune systems, and involving chromosomal instability, cancer predisposition, radiation sensitivity, and cell cycle abnormalities. Studies of the cellular phenotype of A-T have pointed to a defect in a putative system that processes a specific type of DNA damage and initiates a signal transduction pathway controlling cell cycle progression and repair. For a general review of Ataxia-telangiectasia, reference is hereby made to the review Ataxia-Telangiectasis: Closer to Unraveling the Mystery, Eur. J. Hum. Genet. (Shiloh, 1995) which, along with its cited references, is hereby incorporated by reference as well as to the reviews by Harnden (1994) and Taylor et al (1994).

Despite extensive investigation over the last two decades, A-T has remained a clinical and molecular enigma. A-T is a multi-system disease inherited in an autosomal recessive manner, with an average worldwide frequency of 1:40,000-1:100,000 live births and an estimated carrier frequency of 1% in the American population. Notable concentrations of A-T patients outside the United States are in Turkey, Italy and Israel. Israeli A-T patients are Moroccan Jews, Palestinian Arabs, Bedouins and Druzes.

Cerebellar ataxia that gradually develops into general motor dysfunction is the first clinical hallmark and results from progressive loss of Purkinje cells in the cerebellum. Oculocutaneous telangiectasia (dilation of blood vessels) develops in the bulbar conjunctiva and facial skin, and is later accompanied by graying of the hair and atrophic changes in the skin. The co-occurrence of cerebellar ataxia and telangiectases in the conjunctivae and occasionally on the facial skin—the second early hallmark of the disease—usually establishes the differential diagnosis of A-T from other cerebellar ataxias. Somatic growth is retarded in most patients, and ovarian dysgenesis is typical for female patients. Among occasional endocrine abnormalities, insulin-resistant diabetes is predominant, and serum levels of alpha-fetoprotein and carcinoembryonic antigen are elevated. The thymus is either absent or vestigial, and other immunological defects include reduced levels of serum IgA, IgE or IgG2, peripheral lymphopenia, and reduced responses to viral antigens and allogeneic cells, that cause many patients to suffer from recurrent sinopulmonary infections.

Cancer predisposition in A-T is striking: 38% of patients develop malignancies, mainly lymphoreticular neoplasms and leukemias. But, A-T patients manifest acute radiosensitivity, and must be treated with reduced radiation doses, and not with radiomimetic chemotherapy. The most common cause of death in A-T, typically during the second or third decade of life, is sinopulmonary infections with or without malignancy.

The complexity of the disease is reflected also in the cellular phenotype. Chromosomal instability is expressed as increased chromosomal breakage and the appearance in lymphocytes of clonal translocations specifically involving the loci of the immune system genes. Such clones may later become predominant when a lymphoreticular malignancy appears. Primary fibroblast lines from A-T patients show accelerated senescence, increased demand for certain growth factors, and defective cytoskeletal structure. Most notable is the abnormal response of A-T cells to ionizing radiation and certain radiomimetic chemicals. While hypersensitive to the cytotoxic and clastogenic effects of these agents, DNA synthesis is inhibited by these agents to a lesser extent than in normal cells. The concomitant lack of radiation-induced cell cycle delay and reduction of radiation-induced elevation of p53 protein are evidence of defective checkpoints at the G1, S and G2 phases of the cell cycle. The G1 and G2 checkpoint defects are evident as reduced delay in cell cycle progression following treatment with ionizing radiation or radiomimetic chemicals, while the rise in the p53 protein level usually associated in normal cells with radiation-induced G1 arrest is delayed in A-T cells. The defective checkpoint at the S phase is readily observed as radioresistant DNA synthesis (RDS). Increased intrachromosomal recombination in A-T cells was also noted recently. Cellular sensitivity to DNA damaging agents and RDS are usually considered an integral part of the A-T phenotype.

Although these clinical and cellular features are considered common to all “classical” A-T patients, variations have been noted. Milder forms of the disease with later onset, slower clinical progression, reduced radiosensitivity and occasional absence of RDS have been described in several ethnic groups (Fiorilli, 1985; Taylor et al., 1987; Ziv et al., 1989; Chessa et al., 1992). Additional phenotypic variability possibly related to A-T is suggested by several disorders that show “partial A-T phenotype” with varying combinations of ataxia, immunodeficiency and chromosomal instability without telangiectases (12-16) (Ying & Decoteau, 1983; Byrne et al., 1984; Aicardi et al., 1988; Maserati et a;., 1988; Friedman & Weitberg, 1993). Still, other disorders display the A-T phenotype and additional features; most notable is the Nijmegen breakage syndrome that combines A-T features with microcephaly, sometimes with mental retardation, but without telangiectases (Weemaes et al., 1994).

Prenatal diagnoses of A-T using cytogenetic analysis or measurements of DNA synthesis have been reported, but these tests are laborious and subject to background fluctuations and, therefore, not widely used.

A-T homozygotes have two defective copies of the A-T gene and are affected with the disease. A-T heterozygotes (carriers) have one normal copy of the gene and one defective copy of the gene and are generally healthy. When two carriers have children, there is a 25% risk in every pregnancy of giving birth to an A-T affected child.

A-T heterozygotes show a significant excess of various malignancies, with a 3- to 4-fold increased risk for all cancers between the ages of 20 and 80, and a 5-fold increased risk of breast cancer in women. These observations turn A-T into a public health problem and add an important dimension to A-T research, particularly to heterozygote identification. Cultured cells from A-T heterozygotes indeed show an intermediate degree of X-ray sensitivity, but the difference from normal cells is not always large enough to warrant using this criterion as a laboratory assay for carrier detection. The main reason for the unreliability of this assay is the various degrees of overlap between A-T heterozygotes and non-heterozygotes with respect to radiosensitivity. Cytogenetic assays for carriers have the same problems as for prenatal diagnosis, they are labor intensive and not always consistent.

The nature of the protein missing in A-T is unknown. Cell fusion studies have established four complementation groups in A-T, designated A, C, D and E, suggesting the probable involvement of at least four genes or four types of mutations in one gene, with inter-allelic complementation. These four groups are clinically indistinguishable and were found-to account for 55%, 28%, 14% and 3% of some 80 patients typed to date. In Israel, several Moroccan Jewish patients were assigned to group C, while Palestinian Arab patients were assigned to group A.

The general chromosomal localization of the putative A-T gene(s) has been determined, but not the sequence. An A-T locus containing the A-T(A) mutations was localized by Gatti et al. (1988) to chromosome 11, region q22-23, using linkage analysis. The A-T(C) locus was localized by applicant to the same region of chromosome 11, region q22-23, by linkage analysis of an extended Jewish Moroccan A-T family (Ziv et al., 1991). Further studies, conducted by an international consortium in which applicant participated (McConville et al., 1990; Foroud et al., 1991; Ziv et al., 1992), reconfirmed this localization in a series of studies and gradually narrowed the A-T locus to an interval estimated at 4 centimorgan, which probably contains also the A-T(E) mutations.

A proposed gene for complementation group D is disclosed in U.S. Pat. No. 5,395,767 to Murnane et al., issued Mar. 7, 1995. This sequence was found not to be mutated in any complementation group of A-T. Further, the gene sequence was mapped physically distant from the presumptive A-T locus.

Therefore, in order to better understand the nature and effects of A-T, as well as to more accurately and consistently determine those individuals who may carry the defective gene for A-T, it would be advantageous to isolate and determine the gene sequence, mutations of which are responsible for causing A-T, and utilize this sequence as a basis for detecting carriers of A-T and thereby be able to more beneficially manage the underlying conditions and predispositions of those carriers of the defective gene.

### SUMMARY OF THE INVENTION AND ADVANTAGES

According to the present invention, a gene, designated ATM, mutations of which cause ataxia-telangiectasia (A-T) has been purified, isolated and sequenced, as well as mutations of the gene and the genomic organization of the gene has been determined.

The present invention further includes the method for identifying carriers of the defective A-T gene in a population and defective A-T gene products.

The role of the ATM gene in cancer predisposition makes this gene an important target for screening. The detection of A-T mutation carriers is particularly significant in light of their radiation-sensitivity so that carrier exposure to radiation can be properly monitored and avoided.

### BRIEF DESCRIPTION OF THE DRAWINGS

Other advantages of the present invention will be readily appreciated as the same becomes better understood by reference to the following detailed description when considered in connection with the accompanying drawings wherein:

FIGS. 1A-E illustrate the positional cloning steps to identify the A-T gene(s) wherein

FIG. 1A is a high-density marker map of the A-T region on chromosome 11q22-23 (Vanagaite et al., 1995), constructed by generating microsatellite markers within genomic contigs spanning the region and by physical mapping of available markers using the same contigs, the prefix “D11” has been omitted from the marker designations, FDX: the adrenal ferredoxin gene, ACAT: the acetoacetyl-coenzyme A thiolase gene, the stippled box denotes the A-T interval, defined recently by individual recombinants between the markers S1818 and S1819 in a consortium linkage study (Lange et al., 1995), the solid box indicates the two-lod confidence interval for A-T obtained in that study, between S1294 and S384;

FIG. 1B illustrates a part of a YAC contig constructed across this region (Rotman et al., 1994c);

FIG. 1C illustrates part of a cosmid contig spanning the S384-S1818 interval, generated by screening a chromosome-11 specific cosmid library with YAC clones Y16 and Y67, and subsequent contig assembly of the cosmid clones by physical mapping (Shiloh, 1995);

FIG. 1D illustrates products of gene hunting experiments wherein solid boxes denote cDNA fragments obtained by using cosmid and YAC clones for hybrid selection of cDNAs (Lovett et al. 1991; Tagle et al., 1993) from a variety of tissues, open boxes denote putative exons isolated from these cosmids by exon trapping (Church et al., 1993), these sequences hybridized back to specific cosmids (broken lines), which allowed their physical localization to specific subregions of the contig (dotted frames); and

FIG. 1E illustrates a 5.9 kb cDNA clone, designated 7-9 (SEQ ID No: 1), identified in a fibroblast cDNA library using the cDNA fragments and exons in 1D as a probe wherein the open box denotes an open reading frame of 5124 nucleotides, solid lines denote untranslated regions, striped arrowheads denote two Alu elements at the 3′ end, and wherein dotted lines drawn between cDNA fragments and exons the cDNA indicate colinearity of sequences;

FIG. 2 is a diagram of the physical map of the ATM region and relationship to the cDNA wherein the top line represents a linear map of the region containing known genetic markers (the prefix D11 has been omitted from marker designations) and shown below the linear map is a portion of a cosmid contig spanning the region with the arch between ends of cosmids A12 and B4 represents a genomic PCR product, a contig of cDNA clones which span the ATM ORF is shown at the bottom of the figure, broken lines denote the position of specific cDNA sequences with the cosmid contig;

FIG. 3 is a schematic representation of the exon-intron organization of the ATM gene with vertical lines denoting the position of the ATM exons, the 3′ exon and all intronis are drawn to scale; and

FIG. 4 is a schematic representation of 44 A-T mutations along the open reading frame (diagonal lines) of the ATM transcript, wherein for large deletions the symbols mark the locations of the 5′ breakpoints, symbols above the line designate mutations expected to inactivate the ATM protein and symbols below the line designate mutations which a priori may have a milder phenotype, symbols are as follows ● truncations and exon skipping deletions (37), ▾ initiation codon abolished (1), ▪ termination codon abolished (1), ◯ in-frame genomic deleteions and insertions of ≦9bp (4), and □ missense mutations (1).

### DETAILED DESCRIPTION OF THE PREFERRED EMBODIMENT

The present invention consists of a purified, isolated and cloned nucleic acid sequence (SEQ ID Nos: 2,8-10,11-63) encoding a gene, designated ATM, mutations in which cause ataxia-telangiectasia and genetic polymorphisms thereof. The nucleic acid can be genomic DNA, cDNA or mRNA.

The complete coding sequence of the ATM gene is set forth in SEQ ID No: 2 and was submitted to the GenBank database under accession number U33841. There is extensive alternate splicing at the 5′ untranslated region (5′UTR) of the ATM transcript giving rise to twelve different 5′ UTRS. The sequence of the longest 5′UTR is set forth in SEQ ID No: 9. The first exon in this sequence is designated 1b. There is an alternative leader exon, designated 1a (SEQ ID No: 10). The sequence of the complete 3′UTR is set forth in SEQ ID No: 8. Together these sequences contain the complete sequence of the ATM transcript. The genomic sequence for each exon and flanking intronic sequences are set forth in Tables 1 and 5, and SEQ ID Nos: 11-63.

As shown in Example 4, using long-distance PCR, the genomic organization, i.e. structure, of this gene was determined and the exon-intron boundaries identified. The ATM gene spans approximately 150 kb of genomic DNA and consists of 66 (64 plus two alternative exon 1) exons. The initiation codon falls within the fifth exon. The last exon is 3.6 kb long and contains the stop codon and a 3′ untranslated region of about 3800 nucleotides.

The ATM gene is composed of 66 exons (FIG. 3 and Tables 1 and 5). The first two exons are alternatively spliced, and are designated 1a (SEQ ID No: 10) and 1b (SEQ ID No: 9). With the exception of the 3′ exon, ATM exons (SEQ ID Nos: 11-63) range in size from 64 to 372 bp, with an average of 149 bp. The introns vary considerably in size, from 100 bp to about 11 kb, with the majority in the range of 1-3 kb. The consensus dinucleotides GT and AG were found at the donor and acceptor splice sites of all introns, except for a variant donor site with a GC dinucleotide (reviewed in Jackson, 1991) present in the intron 3′ to exon 52. The first methionine of the open reading frame is located in exon 4, whereas the stop codon is located in the 3′ and largest exon of 3.6 kb (Exon 65; SEQ ID No: 63). This exon includes a 3′ untranslated region (UTR)(SEQ ID No: 8) of about 3800 nucleotides.

Polymorphisms are variants in the sequence generally found between different ethnic and geographic locations which, while having a different sequence, produce functionally equivalent gene products.

Current mutation data (as shown in Tables 2 and 3) indicate that A-T is a disease characterized by considerable allelic heterogeneity. Mutations imparting defects into the A-T gene can be point mutations, deletions or insertions. The mutations can be present within the nucleotide sequence of either/or both alleles of the ATM gene such that the resulting amino acid sequence of the ATM protein product is altered in one or both copies of the gene product; when present in both copies imparting ataxia-telangiectasia. Alternatively, a mutation event selected from the group consisting of point mutations, deletions, insertions and rearrangements could have occurred within the flanking sequences and/or regulatory sequences of ATM such that regulation of ATM is altered imparting ataxia-telangiectasia.

Table 2 illustrates ten mutations in the ATM-gene found in A-T patients. Mutations in the ATM gene were found in all of the complementation groups suggesting that ATM is the sole gene responsible for all A-T cases.

Table 3 and FIG. 4 illustrate 54 mutations identified to date in applicant's patient cohort and include 44 new ones and 10 previously listed in Table 2. These mutations were found amongst 55 A-T families: many are unique to a single family, while others are shared by several families, most notably the 4 nt deletion, 7517del4, which is common to 6 A-T families from South-Central Italy. The nature and location of A-T mutations, as set forth in Table 3, provide insight into the function of the ATM protein and the molecular basis of this pleiotropic disease.

This series of 54 A-T mutations is dominated by deletions and insertions. The smaller ones, of less than 12 nt, reflect identical sequence alterations in genomic DNA. Deletions spanning larger segments of the ATM transcript were found to reflect exon skipping, not corresponding genomic deletions. Of the 54 A-T mutations identified, 45 (83.3%) are expected to inactivate the ATM protein by truncating it, by abolishing correct initiation or termination of translation, or by deleting large segments. Additional mutations are four smaller in-frame deletions and insertions, and one substitution of a highly conserved amino acid at the PI 3-kinase domain. The emerging profile of mutations causing A-T is thus dominated by those expected to completely inactivate the ATM protein. ATM mutations with milder effects appear to result in phenotypes related, but not identical, to A-T. In view of the pleiotropic nature of the ATM gene, the range of phenotypes associated with various ATM genotypes may be even broader, and include mild progressive conditions not always defined as clear clinical entities as discussed herein below in Example 3. Screening for mutations in this gene in such cases will reveal wider boundaries for the molecular pathology associated with the ATM gene. The present invention therefore allows the identification of these mutations in subjects with related phenotypes to A-T.

The ATM gene leaves a great deal of room for mutations: it encodes a large transcript. The variety of mutations identified in this study indeed indicates a rich mutation repertoire. Despite this wealth of mutations, their structural characteristics point to a definite bias towards those that inactivate or eliminate the ATM protein. The nature or distribution of the genomic deletions among these mutations do not suggest a special preponderance of the ATM gene for such mutations, such as that of the dystrophin (Anderson and Kunkel, 1992) or steroid sulfatase (Ballabio et al., 1989) genes which are particularly prone to such deletions. Thus, one would have expected also a strong representation of missense mutations, which usually constitute a significant portion of the molecular lesions in many disease genes (Cooper and Krawczak, 1993; Sommer, 1995). However, only two such mutations were identified in the present study. Other point mutations reflected in this series are those that probably underlie the exon skipping deletions observed in many patients, again, exerting a severe structural effect on the ATM protein.

In cloning the gene for A-T (see Example 2), the strategy used was a standard strategy in identifying a disease gene with an unknown protein product known as positional cloning, as is well known in the art. In positional cloning, the target gene is localized to a specific chromosomal region by establishing linkage between the disease and random genetic markers defined by DNA polymorphisms. Definition of the smallest search interval for the gene by genetic analysis is followed by long-range genomic cloning and identification of transcribed sequences within the interval. The disease gene is then identified among these sequences, mainly by searching for mutations in patients.

Several important and long sought disease genes were isolated recently in this way (Collins, 1992; Attree et al., 1992; Berger et al., 1992; Chelly et al., 1993; ,Vetrie et al., 1993; Trofatter et al., 1993; The Huntington's Disease Collaborative Research Group, 1993; The European Polycystic Kidney Disease Consortium, 1994; Miki et al., 1994).

Two complementary methods were used for the identification of transcribed sequences (gene hunting): hybrid selection based on direct hybridization of genomic DNA with cDNAs from various sources (Parimoo et al., 1991; Lovett et al., 1991); and exon trapping (also called exon amplification), which identifies putative exons in genomic DNA by virtue of their splicing capacity (Church et al., 1993). In hybrid selection experiments, cosmid and YAC clones served to capture cross-hybridizing sequences in cDNA collections from placenta, thymus and fetal brain, using the magnetic bead capture protocol (Morgan et al., 1992; Tagle et al., 1993). In parallel experiments, YAC clones were bound to a solid matrix and used to select cDNA fragments from a heterogeneous cDNA collection representing several human tissues (Parimoo et al., 1993). The cosmids were also used for exon trapping with the pSPL3 vector (Church et al., 1994). The captured cDNA fragments and trapped exons were mapped back to the A-T region by hybridization to several radiation hybrids containing various portions of the 11q22-23 region (Richard et al., 1993; James et al., 1994), and to high-density grids containing all the YACs and cosmids spanning this interval. An extensive transcriptional map of the A-T region was thus constructed (Shiloh et al., 1994a).

Pools of adjacent cDNA fragments and exons, expected to converge into the same transcriptional units, were used to screen cDNA libraries. A cluster of 5 cDNA fragments and 3 exons mapped in close proximity to the marker D11S535, where the location score for A-T had peaked (Lange et al., 1995). All these sequences hybridized to the same 5.9 kb of the cDNA clone, 7-9, (SEQ ID No: 1) obtained from a fibroblast cDNA library.

Hybridization of the 7-9 cDNA clone to the radiation hybrid panel indicated that the entire transcript was derived from the chromosome 11 locus. The full sequence of this clone (SEQ ID No: 1) was obtained using a shotgun strategy, and found to contain 5921 bp which includes an open reading frame (ORF) of 5124 nucleotides, a 538 bp 3′ untranslated region (3′ UTR), and a 259 bp 5′ non-coding sequence containing stop codons in all reading frames. (Genbank Accession No. U26455). Two Alu repetitive elements were observed at the 3′ end of this clone and in nine smaller clones representing this gene from the same cDNA library. Since no polyadenylation signal was identified in these cDNA clones, their poly(A) tracts were assumed to be associated with the Alu element rather than being authentic poly(A) tails of these transcripts. This assumption was later supported when applicants identified a cDNA clone derived from the same gene in a leukocyte cDNA library, with an alternative 3′ UTR containing a typical polyadenylation signal. Alignment of the cDNA with the genomic physical map showed that the corresponding gene is transcribed from centromere to telomere.

Hybridization of a probe containing the entire ORF of clone 7-9 to northern blots from various tissues and cell lines revealed a major transcript of 12 kb, later shown to be 13 kb, in all tissues and cell types examined, and minor species of various sizes in several tissues, possibly representing alternatively spliced transcripts of the corresponding gene or other homologous sequences. Genomic sequencing later identified the 5′ non-coding region of clone 7-9 as sequences of the unspliced adjacent intron. Two other cDNA clones from a leukocyte cDNA library were found to contain this intronic sequence in their 5′ ends. These clones may represent splicing intermediates.

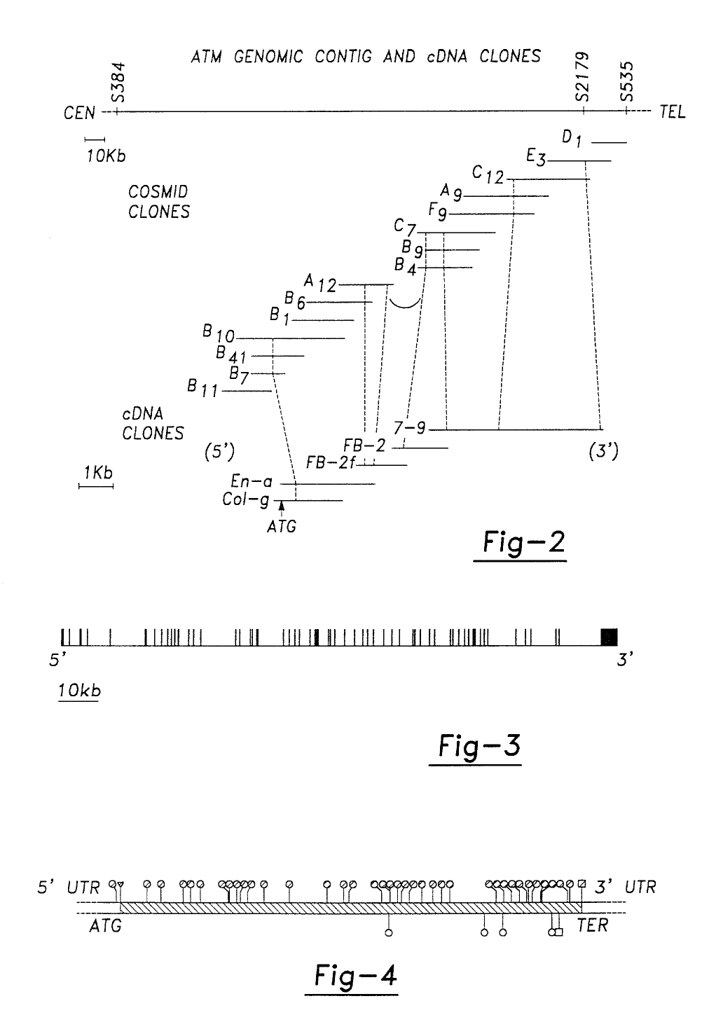

The 7-9 cDNA clone represents only part of the ATM gene transcript. Successive screening of randomly-primed cDNA libraries identified a series of partly overlapping cDNA clones and enabled the construction of a cDNA contig of about 10 Kb (FIG. 2). The gene coding for this transcript spans about 150 Kb of genomic DNA.

The composite cDNA of 9860 bp (GenBank Accession No. U33841; SEQ ID No: 2) includes an open reading frame of 9168 nucleotides, a 538 bp 3′ untranslated region (UTR), and a 164 bp 5′ UTR containing stop codons in all reading frames. The sequence surrounding the first in-frame initiation codon (ACCATGA) resembles the consensus sequence proposed by Kozak et al (1987) for optimal initiation of translation, (A/G)CCATGG. No polyadenylation signal was found at the 3′ UTR. The same poly(A) tail was found in all cDNA clones and 3′ RACE products isolated to date in applicant's laboratory, however, this poly(A) tail most likely belongs to the Alu element contained in the 3′ UTR.

Sequencing and PCR analysis of 32 partial ATM cDNA clones, obtained from 11 cDNA libraries representing 8 different tissues, have been colinear over the coding region, except when they contained unspliced intronic sequences. Thus, alternative splicing within the ATM coding region may not occur, or may take place at a very low frequency, or be restricted to a cell type not yet explored.

The invention further provides a purified protein (SEQ ID No: 3) as encoded by the ATM gene and analogs and mutations thereof (SEQ ID No: 2). The present invention further provides for mutations in SEQ ID No: 3 which cause ataxia-telangiectasia, for example, as set forth in Tables 2 and 3.

Th2 ATM Open Reading Frame (SEQ ID No: 2) product is a large protein of 3056 amino acids, with an expected molecular weight of 350.6 kDa. The ATM gene product (SEQ ID No: 3) contains a PI-3 kinase signature at codons 2855-2875, and a potential leucine zipper at codons 1217-1238. The presence of this leucine zipper may suggest possible dimerization of the ATM protein or interaction with additional proteins. No nuclear localization signal, transmembrane domains or other motifs were observed in this protein sequence.

The ATM gene product is a member of a family of large proteins that share a highly conserved carboxy-terminal region of about 300 amino acids showing high sequence homology to the catalytic domain of PI-3 kinases. Among these proteins are Tellp and Meclp in budding yeast, rad3p in fission yeast, the TOR proteins in yeast and their mammalian counterpart, FRAP (RAFT1), MEI-41 in Drosophila melanogaster, and the catalytic subunit of DNA-dependent protein kinase (DNA-PKcs) in mammals. All of these proteins are implicated in cell cycle control and some of them, like Meclp, rad3p and DNA-PKcs are involved in response to DNA damage (Table 4). The central core of the PI-3 kinase-like domain contains two subdomains with highly conserved residues present in nearly all kinases, including protein and PI-3 kinases. The residues Asp and Asn (at positions 2870 and 2875 in ATM), and the triplet Asp-Phe-Gly (at positions 2889-2891), which represents the most highly conserved short stretch in the protein kinase catalytic domain, have been implicated in the binding of ATP and phosphotransferase activity. Mutations in the genes encoding these proteins result in a variety of phenotypes that share features with A-T, such as radiosensitivity, chromosomal instability, telomere shortening, and defective cell cycle checkpoints (reviewed by Savitsky et al., 1995a and b; Zakian, 1995).

A possible working model for the ATM protein's function is DNA-PK, a serine/threonine protein kinase that is activated in vitro by DNA double-strand breaks and responds by phosphorylating several regulatory proteins (Gottlieb and Jackson, 1994). The ATM protein may be responsible for conveying a signal evoked by a specific DNA damage to various checkpoint systems, possibly via lipid or protein phosphorylation.

The present invention further includes a recombinant protein encoded by SEQ ID No: 2 or SEQ ID No: 3 or analogs thereof. This recombinant protein is isolated and purified by techniques known to those skilled in the art.

An analog will be generally at least 70% homologous over any portion that is functionally relevant. In more preferred embodiments, the homology will be at least 80% and can approach 95% homology to the ATM protein. The amino acid sequence of an analog may differ from that of the ATM protein when at least one residue is deleted, inserted or substituted but the protein remains functional and does not cause A-T. Differences in glycosylation can provide analogs.

The present invention provides an antibody, either polyclonal or monoclonal, which specifically binds to epitopes on the polypeptide/protein encoded by the ATM gene, or mutant epitopes. In preparing the antibody, the protein (with and without mutations) encoded by the ATM gene and polymorphisms thereof is used as a source of the immunogen. Peptide amino acid sequences isolated from the amino acid sequence as set forth in SEQ ID No: 3 or mutant peptide sequences can also be used as an immunogen.

The present invention also provides antibodies against the following peptides:

| | |
|---|---|
| HEPANSSASQSTDLC, | (SEQ ID No:4) |
| CKRNLSDIDQSFDKV, | (SEQ ID No:5) |
| PEDETELHPTLNADDQEC, and | (SEQ ID No:6) |
| CKSLASFIKKPFDRGEVESMEDDTNG. | (SEQ ID No:7) |

The antibodies may be either monoclonal or polyclonal. Conveniently, the antibodies may be prepared against a synthetic peptide based on the sequence, or prepared recombinantly by cloning techniques or the natural gene product and/or portions thereof may be isolated and used as the immunogen. Such proteins or peptides can be used to produce antibodies by standard antibody production technology well known to those skilled in the art as described generally in Harlow and Lane, Antibodies: A Laboratory Manual, Cold Spring Harbor Laborator Press, Cold Spring Harbor, N.Y., 1988.

For producing polyclonal antibodies a host, such as a rabbit or goat, is immunized with the protein or peptide, generally with an adjuvant and, if necessary, coupled to a carrier; antibodies to the protein are collected from the sera.

For producing monoclonal antibodies, the technique involves hyperimmunization of an appropriate donor, generally a mouse, with the protein or peptide fragment and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

The antibody can be bound to a solid support substrate or conjugated with a detectable moiety or be both bound and conjugated as is well known in the art. (For a general discussion of conjugation of fluorescent or enzymatic moieties see Johnstone and Thorpe, Immunochemistry in Practice, Blackwell Scientific Publications, Oxford, 1982.) The binding of antibodies to a solid support substrate is also well known in the art. (see for a general discussion Harlow and Lane Antibodies: A Laboratory Manual, Cold Spring Harbor Laboratory Publications, New York, 1988) The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

The present invention provides vectors comprising an expression control sequence operatively linked to the nucleic acid sequence of the ATM gene, SEQ ID No: 2 and portions thereof as well as mutant sequences which lead to the expression of A-T. The present invention further provides-host cells, selected from suitable eucaryotic and procaryotic cells, which are transformed with these vectors.

Using the present invention, it is possible to transform host cells, including E. coli, using the appropriate vectors so that they carry recombinant DNA sequences derived from the ATM transcript or containing the entire ATM transcript in its normal form or a mutated sequence containing point mutations, deletions, insertions, or rearrangements of DNA. Such transformed cells allow the study of the function and the regulation of the A-T gene. Use of recombinantly transformed host cells allows for the study of the mechanisms of A-T and, in particular it will allow for the study of gene function interrupted by the mutations in the A-T gene region.

Vectors are known or can be constructed by those skilled in the art and should contain all expression elements necessary to achieve the desired transcription of the sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids and other recombination vectors. The vectors can also contain elements for use in either procaryotic or eucaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega at al., Gene Targeting, CRC Press, Ann Arbor, Mich. (1995) and Gilboa et al (1986) and include, for example, stable or transient transfection, lipofection, electroporation and infection with recombinant viral vectors. Introduction of nucleic acids by infection offers several advantages over the other listed methods. Higher efficiency can be obtained due to their infectious nature. See also U.S. Pat. Nos. 5,487,992 and 5,464,764. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

Recombinant methods known in the art can also be used to achieve the sense, antisense or triplex inhibition of a target nucleic acid. For example, vectors containing antisense nucleic acids can be employed to express protein or antisense message to reduce the expression of the target nucleic acid and therefore its activity.

A specific example of DNA viral vector for introducing and expressing antisense nucleic acids is the adenovirus derived vector Adenop53TK. This vector expresses a herpes virus thymidine kinase (TK) gene for either positive or negative selection and an expression cassette for desired recombinant sequences such as antisense sequences. This vector can be used to infect cells that have an adenovirus receptor which includes most cancers of epithelial origin as well as others. This vector as well as others that exhibit similar desired functions can be used to treat a mixed population of cells include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected wits the recombinant virus. An example of such a negative selection marker is the TK gene described above that confers sensitivity to the anti-viral gancyclovir. Negative selection is therefore a means by which infection can be controlled because it provides inducible suicide through the addition of antibiotic. Such protection ensures that if, for example, mutations arise that produce altered forms of the viral vector or sequence, cellular transformation will not occur. Features that limit expression to particular cell types can also be included. Such features include, for example, promoter and regulatory elements that are specific for the desired cell type.

Recombinant viral vectors are another example of vectors useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted. For example, if breast cancer is to be treated, then a vector specific for such epithelial cells should be used. Likewise, if diseases or pathological conditions of the hematopoietic system are to be treated, then a viral vector that is specific for blood cells and their precursors, preferably for the specific type of hematopoietic cell, should be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

If viral vectors are used, for example, the procedure can take advantage of their target specificity and consequently, do not have to be administered locally at the diseased site. However, local administration may provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with the appropriate target specificity for infection.

Transfection vehicles such as liposomes can also be used to introduce the non-viral vectors described above into recipient cells within the inoculated area. Such transfection vehicles are known by one skilled within the art.

The present invention includes the construction of transgenic and knockout organisms that exhibit the phenotypic manifestations of A-T. The present invention provides for transgenic ATM gene and mutant ATM gene animal and cellular (cell lines) models as well as for knockout ATM models. The transgenic model can include those carrying the sequence set forth SEQ ID Nos: 2,8,9 (or 10). These models are constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384,5,175,383, 4,736,866 as well as Burke and Olson, (1991), Capecchi, (1989), Davies et al., (1992), Dickinson et al., (1993), Huxley et al., (1991), Jakobovits et al., (1993), Lamb et al., (1993), Rothstein, (1991), Schedl et al., (1993), Strauss et al., (1993). Further, patent applications WO 94/23049, WO 93/14200, WO 94/06908, WO 94/28123 also provide information. See also in general Hogan et al “Manipulating the Mouse Embryo” Cold Spring Harbor Laboratory Press, 2nd Edition (1994).

According to the present invention, there is provided a method for diagnosing and detecting carriers of the defective gene responsible for causing A-T (see Example 4).

The present invention further provides methods for detecting normal copies of the ATM gene and its gene product. Carrier detection is especially important since A-T mutations underlie certain cases of cancer predisposition in the general population. Identifying the carriers-either by their defective gene or by their missing or defective protein(s) encoded thereby, leads to earlier and more consistent diagnosis of A-T gene carriers. Thus, since carriers of the disease are more likely to be cancer-prone and/or sensitive to therapeutic applications of radiation, better surveillance and treatment protocols can be initiated for them. Conversely, exclusion of A-T heterozygotes from patients undergoing radiotherapy can allow for establishing routinely higher dose schedules for other cancer patients thereby improving the efficacy of their treatment.

Briefly, the methods comprise the steps of obtaining a sample from a test subject, isolating the appropriate test material from the sample and assaying for the target nucleic acid sequence or gene product. The sample can be tissue or bodily fluids from which genetic material and/or proteins are isolated using methods standard in the art. For example, DNA can be isolated from lymphocytes, cells in amniotic fluid and chorionic villi (Llerena et al., 1989).

More specifically, the method of carrier detection is carried out by first obtaining a sample of either cells or bodily fluid from a subject. Convenient methods for obtaining a cellular sample can include collection of either mouth wash fluids or hair roots. A cell sample could be amniotic or placental cells or tissue in the case of a prenatal diagnosis. A crude DNA could be made from the cells (or alternatively proteins isolated) by techniques well known in the art. This isolated target DNA is then used for PCR analysis (or alternatively, Western blot analysis for proteins from a cell line established from the subject) with appropriate primers derived from the gene sequence by techniques well known in the art. The PCR product would then be tested for the presence of appropriate sequence variations in order to assess genotypic A-T status of the subject.

The specimen can be assayed for polypeptides/proteins by immunohistochemical and immunocytochemical staining (see generally Stites and Terr, Basic and Clinical Immunology, Appleton and Lange, 1994), ELISA, RIA, immunoblots, Western blotting, immunoprecipitation, functional assays and protein truncation test. In preferred embodiments, Western blotting, functional assays and protein truncation test (Hogervorst et al., 1995) will be used. mRNA complementary to the target nucleic acid sequence can be assayed by in situ hybridization, Northern blotting and reverse transcriptase-polymerase chain reaction. Nucleic acid sequences can be identified by in situ hybridization, Southern blotting, single strand conformational polymorphism, PCR amplification and DNA-chip analysis using specific primers. (Kawasaki, 1990; Sambrook, 1992; Lichter et al, 1990; Orita et al, 1989; Fodor et al., 1993; Pease et al., 1994)

ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, 1992.

Current mutation data (as shown in Tables 2 and 3) indicate that A-T is a disease characterized by considerable allelic-heterogenicity. It is not surprising that there are hundreds (or even thousands) of ATM mutations (as is the case for cystic fibrosis and BRCA1) as shown in Table 3. Thus, it will be important for a successful mutation Screen to be able to detect all possible nucleotide alterations in the ATM gene, rather than being focused on a limited subset (see Example 4). Methods including direct sequencing of PCR amplified DNA or RNA or DNA chip hybridization Wodor et al., 1993; Pease et al., 1994) can be applied along with other suitable methods known to those skilled in the art.

In order to use the method of the present invention for diagnostic applications, it is advantageous to include a mechanism for identifying the presence or absence of target polynucleotide sequence (or alternatively proteins). In many hybridization based diagnostic or experimental procedures, a label or tag is used to detect or visualize for the presence or absence of a particular polynucleotide sequence. Typically, oligomer probes are labelled with radioisotopes such as ³²P or ³⁵S (Sambrook, 1992) which can be detected by methods well known in the art such as autoradiography. Oligomer probes can also be labelled by non-radioactive methods such as chemiluminescent materials which can be detected by autoradiography (Sambrook, 1992). Also, enzyme-substrate based labelling and detection methods can be used. Labelling can be accomplished by mechanisms well known in the art such as end labelling (Sambrook, 1992), chemical labelling, or by hybridization with another labelled oligonucleotide. These methods of labelling and detection are provided merely as examples and are not meant to provide a complete and exhaustive list of all the methods known in the art.

The introduction of a label for detection purposes can be accomplished by attaching the label to the probe prior to hybridization.

An alternative method for practicing the method of the present invention includes the step of binding the target DNA to a solid support prior to the application of the probe. The solid support can be any material capable of binding the target DNA, such as beads or a membranous material such as nitrocellulose or nylon. After the target DNA is bound to the solid support, the probe oligomers is applied.

Functional assays can be used for detection of A-T carriers or affected individuals. For example, if the ATM protein product is shown to have PI 3-kinase or a protein kinase biochemical activity which can be assayed in an accessible biological material, such as serum, peripheral leukocytes, etc., then homozygous normal individuals would have approximately normal biological activity and serve as the positive control. A-T carriers would have substantially less than normal-biological activity, and affected (i.e. homozygous) individuals would have even less biological activity and serve as a negative control. Such a biochemical assay currently serves as the basis for Tay-Sachs carrier detection.

The present invention provides a method for a rapid and efficient method to identify any mutations in small amounts of RNA, such as those that are obtained from as little as 100 μl of peripheral blood. RNA is extracted from the sample, using the Tri-Reagent system (Molecular Research Center, Cincinnati Ohio) or other equivalent method and it is subjected to reverse transcription as described by Gilad et al (1996). A selected open reading frame is then amplified using RT-PCR as described herein below, and the resulting products are further amplified using nested PCR primers. The products of these reactions are then subjected to restriction endonuclease fingerprinting (REF) as described hereinbelow.

This method uses the cDNA obtained from the RNA for only one PCR, the products of which later serve for further analysis. Therefore, only minimal amounts of RNA need be extracted, reducing costs.

Using this method RNA is extracted from a sample and subjected to reverse transcription (Gilad et al., 1996). The entire ATM open reading frame (SEQ ID No: 2) is then amplified using RT-PCR as described herein below, and the resulting products are further amplified using nested PCR primers (SEQ ID Nos: 82-91; see herein below). The products of these reactions are then subjected to restriction endonuclease fingerprinting (REF) as described hereinbelow.

However, when only genomic DNA (as for example old tumor specimens) is available the preferred embodiment to undertake mutation analysis requires individual amplification of exons or groups of exons. To undertake this analysis primer pairs have been developed based on the information set forth in Table 1 and in Table 5 which is a further amplification of Table 1. Primers are selected from the flanking sequences using standard computer algorithms as are known in the art. Shown below are five examples of primer pairs which can be used for the analysis.

| | |
|---|---|
| Primers Exons 4 and 5 | |
| 5′primer: | |
| 5′-cacacctctttctctctatatatg-3′ | (SEQ ID No:82) |
| 3′primer: | |
| 5′-cacacaaaagtaatatcacaacag-3′ | (SEQ ID No:83) |
| Primers Exon 17 | |
| 5′primer: | |
| 5′-gtatgtccaagatcaaagtacac-3′ | (SEQ ID No:84) |
| 3′primer: | |
| 5′-ggtgacagagaaagatcctatctc-3′ | (SEQ ID No:85) |
| Primers Exon 25 | |
| 5′primer: | |
| 5′-ctggaatatgctttggaaagtagg-3′ | (SEQ ID No:86) |
| 3′primer: | |
| 5′-ccaaacttggtgaagtaatttatgg-3′ | (SEQ ID No:87) |
| Primers Exon 34 | |
| 5′primer: | |
| 5′-cacaggcttaaccaatacgtg-3′ | (SEQ ID No:88) |
| 3′primer: | |
| 5′-caggtagaaatagcccatgtc-3′ | (SEQ ID No:89) |
| Primers Exon 46 | |
| 5′primer: | |
| 5′-gtcctttggtgaagctatttatac-3′ | (SEQ ID No:90) |
| 3′primer: | |
| 5′-ccaagtctttatctcttcatcaatgc-3′ | (SEQ ID No:91) |

The present invention also provides a kit for diagnosis and detection of the defective A-T gene in a population. in general the population will be one that has been characterized for ATM mutations. For example Moroccan Jews in which only one mutation is known, or a population such as the Amish in which also one mutation is known. Each kit would be customized to the population being screened. The kit includes a molecular probe(s) complementary to genetic sequences of the defective gene which causes ataxia-telangiectasia (A-T) in that population and suitable labels for detecting hybridization of the molecular probe and the defective gene thereby indicating the presence of the defective gene. The molecular probe has a DNA sequence complementary to mutant sequences. Alternatively, the kit can contain reagents and antibodies for detection of mutant proteins.

The above discussion provides a factual basis for the use and identification of the ataxia-telangiectasia gene and gene products and identification of carriers as well as construction of transgenic organisms. The methods used in the present invention can be shown by the following non-limiting example and accompanying figures.

### EXAMPLES

Materials and Methods:

General methods in molecular biology: Standard molecular biology techniques known in the art and not specifically described were generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989). Protein analysis techniques are as generally described in Coligan et al., Current Protocols in Immunology, John Wiley and Sons, Baltimore, Md. (1992, 1994).

RT-PCR

Following reverse transcription (Gilad et al., 1996), RT-PCR is performed in 25 μl reaction mix. One tenth (1 1) of the reverse transcription products are added to the reaction mix. The reaction mix contains 2U of Expand Long Template PCR System (Boehringer Mannheim, Mannheim, Germany), in an appropriate buffer containing 1.75 mM MgCl₂, 1.5 μg of Antitaq mAb (Chimerx), 0.2 mM dNTPs (Pharmacia) and 1 mM of each of the following primers:

a. 5′-gtgcagtgaggcatacatcac-3′ (SEQ ID No: 64)

b. 5′-aaggctgaatgaaagggtaattc-3′ (SEQ ID No: 65)

PCR Conditions

After denaturation for 3 minutes at 94° C., 5 cycles of amplification are performed as follows: 20 seconds at 93° C., 1 minute at 64° C. and 8 minutes at 68° C., followed by 30 cycles of 20 seconds at 93° C., 1 minute at 60° C. and 7.5 minutes at 68° C. Final extension is then performed for 10 minutes at 68° C.

Restriction Endonuclease Fingerprinting (REF)

The open reading frame of the ATM transcript was divided into eight partly overlapping PCR fragments. Each fragment spans 1.0-1.6 kb and is defined by two PCR primers as follows.

| | |
|---|---|
| REF1. | |
| a. 5′-caccctgctgcccagatatg-3′ | (SEQ ID No:66) |
| b. 5′-cttataccacgaaaggtaatacac-3′ | (SEQ ID No:67) |
| REF2. | |
| a. 5′-gaggtcaaacctagaaagctcac-3′ | (SEQ ID No:68) |
| b. 5′-cctctcctttgttagatgcc-3′ | (SEQ ID No:69) |
| REF3. | |
| a. 5′-ctaggtcaaagcaatatggactc-3′ | (SEQ ID No:70) |
| b. 5′-catgcgatggaaaatgaggtg-3′ | (SEQ ID No:71) |
| REF4. | |
| a. 5′-cagagattgtggtggagttattg-3′ | (SEQ ID No:72) |
| b. 5′-gcattatgaaggtccactgaag-3′ | (SEQ ID No:73) |
| REF5. | |
| a. 5′-cttcagtggaccttcataatgc-3′ | (SEQ ID No:74) |
| b. 5′-ccatacaaactatctggctcc-3′ | (SEQ ID No:75) |
| REF6. | |
| a. 5′-ctggaataagtttacaggatcttc-3′ | (SEQ ID No:76) |
| b. 5′-gatgatttcatgtagttttcaattc-3′ | (SEQ ID No:77) |
| REF7. | |
| a. 5′-gatggagaaagtagtgatgagc-3′ | (SEQ ID No:78) |
| b. 5′-agtcaccagatttccatattctc-3′ | (SEQ ID No:79) |
| REF8. | |
| a. 5′-aagatgttgttgtccctactatg-3′ | (SEQ ID No:80) |
| b. 5′-aaggctgaatgaaagggtaattc-3′ | (SEQ ID No:81) |

Patient and family resources: A cell line repository was established containing 230 patient cell lines and 143 cell lines from healthy members of Moroccan Jewish, Palestinian Arab and Druze families. Some of these pedigrees are highly inbred and unusually large (Ziv et al., 1991; Ziv, 1992). In view of the large number of meiotic events required for high-resolution linkage analysis, applicants collaborated with Dr. Carmel McConville (University of Birmingham, UK) and Dr. Richard Gatti (UCLA, Los Angeles, Calif.), who have also established extensive repositories of A-T families. Linkage analysis was conducted on a pool of 176 families.

### Example 1

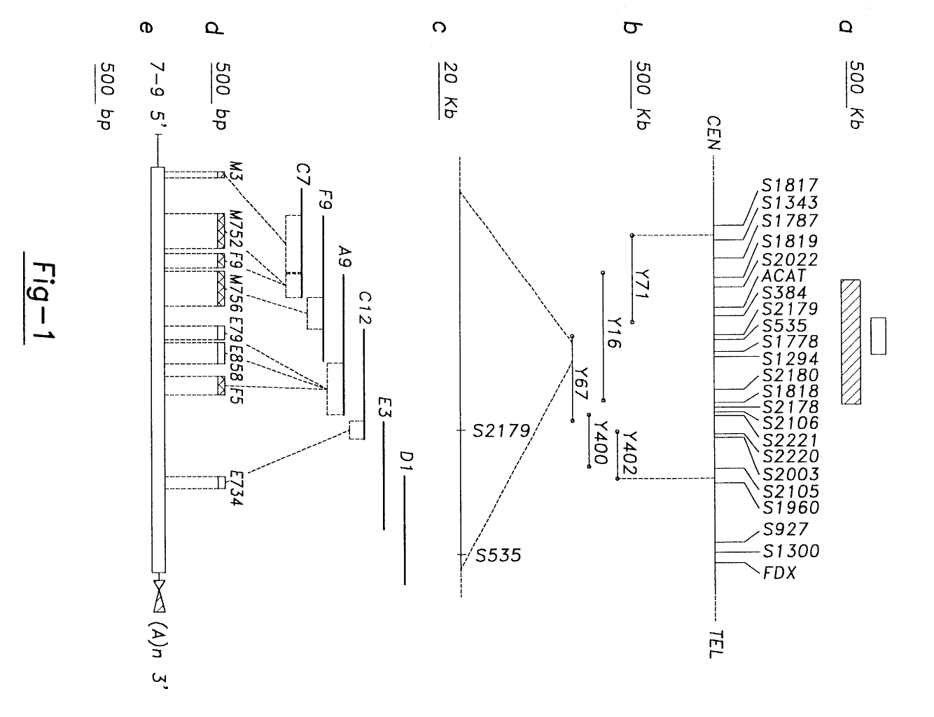

Definition of the A-T interval by genetic analysis: Studies based only on analysis of Israeli A-T families enabled localization of the A-T(C) gene at 11q22-23 (Ziv, 1991), and confirmed the localization of A-T(A) mutation in Palestinians to the same region (Ziv et al., 1992). Studies with the Birmingham group further narrowed the major A-T interval to 4 centimorgans, between D11S611 and D11S1897 (McConville et al., 1993), and subsequently to 3 centimorgans, between GRIA4 and D11S1897 (Ambrose et al., 1994a; McConville et al., 1994) (see also Shiloh, 1995, and FIG. 1).

All these studies were conducted with biallelic markers, whose power is limited by their low polymorphic information content (PIC). The recently discovered microsatellite markers based on variable numbers of tandem simple repeats (Litt and Luty, 1989; Weber and May, 1989) are much more powerful due to their high degree of polymorphism. Microsatellite markers were used to saturate the A-T region using two approaches. The first, was based on physical mapping of microsatellite markers generated by others which were loosely linked to chromosome 11q.

Mapping experiments were conducted using YAC and cosmid contigs which allowed precise, high-resolution localization of DNA sequences in this region of chromosome 11. These experiments led to the localization of 12 microsatellites at the A-T region (Vanagaite et al., 1994a; Vanagaite et al., 1995).

The second approach was based on generating new microsatellites within the YAC contig. A rapid method for the identification of polymorphic CA-repeats in YAC clones was set up (Rotman, 1995) resulting in the generation of twelve new markers within the A-T locus (Vanagaite et al., 1995; Rotman et al., 1995; Rotman et al., 1994b). Hence, the high-density microsatellite map constructed in this manner contained a total of 24 new microsatellite markers and spans the A-T locus and flanking sequences, over a total of six megabases (Vanagaite et al., 1995).

Repeated linkage analysis on the entire cohort of A-T families indicated that the A-T(A) locus was definitely located within a 1.5 megabase region between D11S1819 and D11S1818 (Gatti et al., 1994) as shown in FIG. 1 and in Shiloh (1995), with a clear peak of the cumulative lod score under D11S535 (Lange et al., 1994).

Concomitant with these studies, linkage disequilibrium (LD) analysis of Moroccan-Jewish A-T patients was conducted. LD refers to the non-random association between alleles at two or more polymorphic loci (Chakravarti et al., 1984). LD between disease loci and linked markers is a useful tool for the fine localization of disease genes (Chakravarti et al., 1984; Kerem et al. 1989; Ozelius et al., 1992; Sirugo et al., 1992; Hastbacka et al., 1992; Mitchison et al., 1993). LD is particularly powerful in isolated ethnic groups, where the number of different mutations at a disease locus is likely to be low (Hastbacka et al., 1992; Lehesjoki et al., 1993; Aksentijevitch et al., 1993). Early on, applicants observed very significant LD (p<0.2−p<0.001) between A-T and markers along the D11S1817-D11S927 region in the patients of the sixteen Moroccan-Jewish A-T families identified in Israel (Oskato et al., 1993). Further analysis with the new markers narrowed the peak of linkage disequilibrium to the D11S384-D11S1818 region as shown in FIG. 1.

Haplotype analysis indicated that all of the mutant chromosomes carry the same D11S384-D11S1818 haplotype, suggesting a founder effect for A-T in this community, with one mutation predominating.

### Example 2

### Sequencing the ATM Gene

Cloning the disease locus in a contig (set of overlapping clones) was essential in isolating the A-T disease gene. The entire A-T locus and flanking region in a contig of yeast artificial chromosomes (YACs) was cloned by methods well known in the art (Rotman et al. 1994c; Rotman et al., 1994d). This contig was instrumental in the construction of the microsatellite map of the region (Vanagaite et al., 1995) and subsequently enabled construction of cosmid contigs extending over most of the interval D11S384-D11S1818. Cosmids corresponding to the YAC clones were identified in a chromosome 11-specific cosmid library supplied by Dr. L. Deaven (Los Alamos National Laboratory) and were ordered into contigs by identifying overlaps as shown in FIG. 1.

Isolation of the A-T gene: Transcribed sequences were systematically identified based on two complementary methods:

1. Use of an improved direct selection method based on magnetic bead capture (MBC) of cDNAs corresponding to genomic clones (Morgan et al., 1992; Tagle et al., 1993). In several, large-scale experiments YAC or cosmid DNA was biotinylated and hybridized to PCR-amplified cDNA from thymus, brain and placenta. Genomic DNA-cDNA complexes were captured using streptavidin-coated magnetic beads which was followed with subsequent elution, amplification, and Cloning of captured cDNAs. The cDNA inserts were excised from a gel, self-ligated to form concatamers and sonicated to obtain random fragments. These fragments were size fractionated by gel electrophoresis, and the 1.0-1.5 Kb fraction-was extracted from the gel and subcloned in a plasmid vector. The end portions of individual clones were sequenced using vector-specific primers, in an automated sequencer (Model 373A, Applied Biosystems), and the sequences were aligned using the AutoAssembler program (Applied Biosystems Division, Perkin-Elmer Corporation). In the final sequence each nucleotide position represents at least 3 independent overlapping readings.

YACs were also used and were no less efficient than cosmids as starting material for MBC, with more than 50% of the products mapping back to the genomic clones. However, when a small panel of radiation hybrids spanning the A-T region was used to test the cDNA fragments, it was found that some clones that hybridized back to the YACs and cosmids were not derived from this region. This pitfall probably stems from limited homology between certain portions of different genes, and points up the necessity to use radiation hybrid mapping when testing the authenticity of the captured sequences, and not to rely solely on cloned DNA for this purpose.

Homology searches in sequence databases showed that only one of the first 105 cDNA fragments mapped to the A-T region was homologous to a sequence previously deposited in one of the databases, as an expressed sequence tag (EST).

2. Exon amplification, also termed “exon trapping” (Duyk et al., 1990; Buckler et al., 1991), is based on cloning genomic fragments into a vector in which exon splice sites are flagged by splicing to their counterpart sites in the vector. This method of gene identification was expected to complement the MBC strategy, since it does not depend on the constitution of cDNA libraries or on the relative abundance of transcripts, and is not affected by the presence of repetitive sequences in the genomic clones. An improved version of this system (Church et al., 1993) that eliminated problems identified in an earlier version, including a high percentage of false positives and the effect of cryptic splice sites was utilized. Each experiment ran a pool of three to five cosmids with an average of two to five exons identified per cosmid. A total of forty five exons were identified.

Sequence analysis and physical mapping indicated that MBC and exon amplification were complementary in identifying transcribed sequences.

The availability of a deep cosmid contig enabled rapid and precise physical localization of the cDNA fragments and captured exons, leading to a detailed transcriptional map of the A-T region.

Both MBC and exon amplification yielded short (100-1000 bp) transcribed sequences. Those sequences were used as anchor points in isolating full-length clones from twenty eight cDNA libraries currently at applicants disposal and which represented a variety of tissues and cell lines.

Initial screening of the cDNA libraries by polymerase chain reaction (PCR) using primer sets derived from individual cDNA fragments or exons aided in the identification of the libraries most likely to yield corresponding cDNA clones.

Large scale screening experiments were carried out in which most of the cDNA fragments and exons were used in large pools. In addition to the mass screening by hybridization, PCR-based screening methods and RACE (rapid amplification of cDNA ends) (Frohman et al., 1988; Frohman et al., 1994) was employed to identify full-length cDNAs.

The above experiments resulted in the initial identification and isolation of a cDNA clone designated 7-9 (Savitsky et al, 1995a), the complete sequence of which is set forth in SEQ ID No: 1 and which is derived from a gene located under the peak of cumulative location score obtained by linkage analysis as shown in FIG. 1. The gene extends over some 300 kilobases (kb) of genomic DNA and codes for two major mRNA species of 12 kb and 10.5 kb in length. The 7-9 clone is 5.9 kb in length and, therefore, is not a full length clone.

An open reading frame of 5124 bp within this cDNA encodes a protein with signature motifs typical of a group of signal transduction proteins known as phosphatidylinositol 3-kinases (PI 3-kinases). PI 3-kinases take part in the complex system responsible for transmitting signals from the outer environment of a cell into the cell. It is not clear yet whether the protein product of the corresponding gene encodes a lipid kinase or a protein kinase.

The gene encoding the 7-9 cDNA clone was considered a strong A-T candidate and mutations were sought in patients. Southern blotting analysis revealed a homozygous deletion in this gene in affected members of Family N., an extended Palestinian Arab A-T family which has not been assigned to a specific complementation group. All the patients in this family are expected to be homozygous by descent for a single A-T mutation. The deletion includes almost the entire genomic region spanned by transcript 7-9, and was found to segregate in the family together with the disease. This finding led to a systematic search for mutations in the 7-9 transcript in additional patients, especially those previously assigned to specific complementation groups.

The restriction endonuclease fingerprinting (REF) method (Liu and Sommer 1995) was applied to reverse-transcribed and PCR-amplified RNA (RT-PCR) from A-T cell lines. Observation of abnormal REF patterns was followed by direct sequencing of the relevant portion of the transcript and repeated analysis of another independent RT product. In compound heterozygotes, the two alleles were separated by subcloning of RT-PCR products and individually sequenced. Genomic sequencing was conducted in some cases to confirm the sequence alteration at the genomic level. Additional family members were studied when available.

Initially, ten sequence alterations (Table 2) were identified in the 7-9 transcript in 13 A-T patients including two sibling pairs. Most of these sequence changes are expected to lead to premature truncation of the protein product, while the rest are expected to create in-frame deletions of 1-3 amino acid residues in this protein. While the consequences of the in-frame deletions remain to be investigated, it is reasonable to assume that they result in impairment of protein function. In one patient, AT3NG, the loss of a serine residue at position 1512 occurs within the PI3-kinase signature sequence. This well conserved domain is distantly related to the catalytic site of protein kinases, hence this mutation is likely to functionally affect the 7-9 protein.

In view of the strong evidence that mutations in this gene are responsible for A-T, it was designated ATM (A-T, Mutated). Since these patients represent all complementation groups of the disease and considerable ethnic variability, these results indicate that the ATM gene alone is responsible for all A-T cases.

In order to complete the cloning of the entire ATM open reading frame, fetal brain and colon random-primed libraries obtained from Stratagene (San Diego, Calif.) and an endothelial cell random-primed library (a gift of Dr. David Ginsburg, University of Michigan) were screened. A total of 1×10⁶ pfu were screened at a density of 40,000 pfu per 140 mm plate, and replicas were made on Qiabrane filters (Qiagen), as recommended by the manufacturer. Filters were prehybridized in a solution containing 6×SSC, 5×Denhardt's, 1% N-laurylsarcosyl, 10% dextran sulfate and 100 μg/ml salmon sperm DNA for 2 hours at 65° C. Hybridization was performed for 16 hrs under the same conditions with 1×10⁶ cpm/ml of ³²p-labelled probe, followed by final washes of 30 minutes in 0.25×SSC, 0.1KSDS at 60° C. Positive clones were plaque-purified using standard techniques and sequenced. DNA sequencing was performed using an automated DNA sequencer (Applied Biosystems, model 373A), and the sequence was assembled using the AutoAssembler program (Applied Biosystems Division, Perkin-Elmer Corporation). In the final sequence, each nucleotide represents at least four independent readings in both directions.

Database searches for sequence similarities were performed using the BLAST network service. Alignment of protein sequences and pairwise comparisons were done using the MACAW program, and the PILEUP and BESTFIT programs in the sequence analysis software package developed by the Genetics Computer Group at the University of Wisconsin.

### Example 3

### Detection of Mutations

Determination of mutations: The recently discovered ATM gene is probably involved in a novel signal transduction system that links DNA damage surveillance to cell cycle control. A-T mutations affect a variety of tissues and lead to cancer predisposition. This striking phenotype together with the existence of “partial A-T phenotypes” endow the study of ATM mutations with special significance.

Materials and Methods.

RT-PCR: Total RNA was extracted from cultured fibroblast or lymphoblast cells using the Tri-Reagent system (Molecular research Center, Cincinnati, Ohio). Reverse transcription was performed on 2.5 ug of total RNA in a final volume of 10 ul, using the Superscript II Reverse Transcriptase (Gibco BRL, Gaithersburg, Md.) in the buffer recommended by the supplier, and in the presence of 125 U/ml of RNAsin (Promega) and 1 mM dNTPs (Pharmacia). Primers were either oligo(dT) (Pharmacia) or a specifically designed primer. The reaction products were used as templates for PCR performed with specific primers. These reactions were carried out in 50 μl containing 2 units of Taq DNA Polymerase (Boehringer Mannheim, Mannheim, Germany), 200 μM dNTPs, 0.5 μM of each primer, and one tenth of the RT-PCR products. The products were purified using the QIA-quick spin system (Qiagen, Hilden, Germany).

Restriction endonuclease fincerorinting: The protocol of Liu and Sommer (1995) was followed with slight modifications. RT-PCR was performed as described above, using primers defining PCR products of 1.0-1.6 kb. One hundred ng of amplified DNA was digested separately with 5 or 6 restriction endonucleases in the presence of 0.2 units of shrimp alkaline phosphatase (United States Biochemicals, Cleveland, Ohio). Following heat inactivation at 65° C. for 10 minutes, the digestion products corresponding to the same PCR product were pooled, denatured at 96° C. for 5 minutes and immediately chilled on ice. Ten ng of this fragment mixture was labeled in the presence of 6 μCi of [γ-³³P)ATP and 1 unit of T4 polynucleotide kinase (New England Biolabs, Beverly, Mass.) at 37° C. for 45 minutes. Twenty μl of stop solution containing 95% formamide, 20 mM EDTA, 0.05% bromophenol blue, 0.05% xylene cyanol, and 10 mM NaOH were added, and the samples were boiled for 3 minutes and quick-chilled on ice. Electrophoresis was performed in 5.6% polyacrylamide gels in 50 mM Tris-borate, pH 8.3, 1 mM EDTA at constant power of 12 W for 3 hours at room temperature, with a fan directed to the glass plates, keeping them at 22-24° C. The gels were dried and subjected to autoradiography.

Direct sequencing of PCR products: Five hundred ng of PCR products was dried under vacuum, resuspended in reaction buffer containing the sequencing primer, and the mixture was boiled and snap-frozen in liquid nitrogen. The Sequenase II system (Unites States Biochemicals) was used to carry out the sequencing reaction in the presence of 0.5 μg of single-strand binding protein (T4 gene 32 protein, United States Biochemicals). The reaction products were treated with 0.1 μg-of proteinase K-at 65° C. for 15 minutes, separated on a 6% polyacrylamide gel, and visualized by autoradiography.

Using the methods described herein above the ATM transcript was scanned for mutations in fibroblast and lymphoblast cell lines derived from an extended series of A-T patients from 13 countries, all of whom were characterized by the classical A-T phenotype. The analysis was based on RT-PCR followed by restriction endonuclease fingerprinting (REF). REF is a modification of the single-strand conformation polymoprphism (SSCP) method, and enables efficient detection of sequence alterations in DNA fragments up to 2 kb in length (Liu and Sommer, 1995).

Briefly, after PCR amplification of the target region, multiple restriction endonuclease digestions are performed prior to SSCP analysis, in order to increase the sensitivity of the method and enable precise localization of a sequence alteration within the analyzed fragment. The coding sequence of the ATM transcript, which spans 9168 nucleotides (SEQ ID No: 2) (Savitsky et al., 1995b), was thus divided into 8 partly overlapping portions of 1.0-1.6 Kb, and each one was analyzed separately (see Example 4 also). Sequence alterations causing abnormal REF patterns were located and disclosed by direct sequencing. Mutations identified in this way were reconfirmed by repeating the RT-PCR and sequencing, or by testing the presence of the same mutations in genomic DNA.

In compound heterozygotes, the two alleles were separated by subcloning and individually sequenced. In some cases, agarose gel electrophoresis showed large deletions in the ATM transcript manifested as RT-PCR products of reduced sizes. The breakpoints of such deletions were delineated by direct sequencing of these products.

The 54 mutations identified to date in our patient cohort (Table 3, FIG. 4) include 44 new ones and 10 previously identified ones (Table 2). (Mutations in Table 3 are presented according to the nomenclature proposed by Beaudet & Tsui (1993); nucleotide numbers refer to their positions in the sequence of the ATM transcript (accession number U33841); the first nucleotide of the open reading frame was designated +1.) These mutations were found amongst 65 A-T families: many are unique to a single family, while others are shared by several families, most notably the 4 nt deletion, 7517del4, which is common to 6 A-T families from South-Central Italy (Table 3). According to this sample, there is a considerable heterogeneity of mutations in A-T, and most of them are “private”. The proportion of homozygotes in this sample is relatively high due to a high degree of consanguinity the populations studied. It should be noted, however, that apparently homozygous patients from non-consanguineous families may in fact be compound heterozygotes with one allele not expressed.

This series of 54 A-T mutations is dominated by deletions and insertions. The smaller ones, of less than 12 nt, reflect identical sequence alterations in genomic DNA. Deletions spanning larger segments of the ATM transcript were found to reflect exon skipping, not corresponding genomic deletions. This phenomenon usually results from sequence alterations at splice junctions or within introns, or mutations within the skipped exons, mainly of the nonsense type (Cooper and Krawczak, 1993; Sommer, 1995; Steingrimsdottir et al., 1992; Gibson et al., 1993; Dietz and Kendzior, 1994). One large deletion spans about 7.5 Kb of the transcript and represents a genomic deletion of about 85 Kb within the ATM gene. Of these deletions and insertions, 25 are expected to result in frameshifts. Together with the 5 nonsense mutations, truncation mutations account for 83% of the total number of mutations in this sample. Nine in-frame deletions span long segments (30-124 aa) of the protein, and similarly to the truncation mutations, are expected to have a severe effect on the protein's structure. It should be noted that two base substitutions abolish the translation initiation and termination codons. The latter is expected to result in an extension of the ATM protein by an additional 29 amino acids. This mutation may affect the conformation of the nearby PI 3-kinase-like domain.

While the effect of the 5 small (1-3 aa) in-frame deletions and insertions on the ATM protein remains to be studied, it should be noted that one such deletion (8578del3) leads to a loss of a serine residue at position 2860. This amino acid is part of a conserved motif within the PI 3-kinase-like domain typical of the protein family to which ATM is related, and is present in 7 of 9 members of this family. The single missense mutation identified in this study, which leads to a Glu2904Gly substitution, results in a nonconservative alteration of another extremely conserved residue within this domain, which is shared by all of these proteins. The patient homozygous for this mutation, AT41RM, shows the typical clinical A-T phenotype. Measurement of radioresistant DNA synthesis in the patient's cell line revealed a typical A-T response, demonstrating that this patient has the classical A-T cellular phenotype.

As discussed herein above, the ATM gene of the present invention is probably involved in a novel signal transduction system that links DNA damage surveillance to cell cycle control. A-T mutations affect a variety of tissues and lead to cancer predisposition. This striking phenotype together with the existence of “partial A-T phenotypes” endow the study of ATM mutations with special significance.

The ATM gene leaves a great deal of room for mutations: it encodes a large transcript. The variety of mutations identified in this study indeed indicates a rich mutation repertoire. Despite this wealth of mutations, their structural characteristics point to a definite bias towards those that inactivate or eliminate the ATM protein. The nature or distribution of the genomic deletions among these mutations do not suggest a special preponderance of the ATM gene for such mutations, such as that of the dystrophin (Anderson and Kunkel, 1992) or steroid sulfatase (Ballabio et al., 1989) genes which are particularly prone to such deletions. Thus, one would have expected also a strong representation of missense mutations, which usually constitute a significant portion of the molecular lesions in many disease genes (Cooper and Krawczak, 1993; Sommer, 1995). However, only two such mutations were identified in the present study. Other point mutations reflected in this series are those that probably underlie the exon skipping deletions observed in many patients, again, exerting a severe structural effect on the ATM protein.

A technical explanation for this bias towards deletions and insertions could be a greater ability of the REF method to detect such lesions versus its ability to detect base substitution. Liu and Sommer (1995) have shown, however, that the detection rate of this method in a sample of 42 point mutations in the factor IX gene ranged between 88% and 100%, depending on the electrophoresis conditions. The 10 base substitutions detected directly by the REF method in the present study (Tables 2 and 3), indicate that such sequence alterations are detected in our hands as well.

Since the expected result of most of these mutations is complete inactivation of the protein, this skewed mutation profile might represent a functional bias related to the studied phenotype, rather than a structural feature of the ATM gene that lends itself to a particular mutation mechanism. The classical A-T phenotype appears to be caused by homozygosity or compound heterozygosity for null alleles, and hence is probably the most severe expression of defects in the ATM gene. The plethora of missense mutations expected in the large coding region of this gene is probably rarely represented in patients with classical A-T, unless such a mutation results in complete functional inactivation of the protein. By inference, one missense identified in this study, Glu2940Gly, which substitutes a conserved amino acid at the PI 3-kinase domain and clearly gives rise to a classical A-T phenotype, points to the importance of this domain for the biological activity of the ATM protein. Mutations in this domain abolish the telomere-preserving function-of the TEL1 protein in S. cerevisiae (Greenwell et al., 1995), a protein which shows a particularly high sequence similarity to ATM (Savitsky et al., 1995b; Zakian, 1995). Another member of the family of PI 3-kinase-related proteins that includes ATM is the mammalian FRAP. Mutations in the PI 3-kinase domain abolish its autophosphorylation ability and biological activity (Brown et al., 1995). These observations, together with the mutation shown here, suggest that this domain in ATM is also likely to include the catalytic site, which may function as a protein kinase.

Genotype-phenotype relationships associated with the ATM gene appear therefore to extend beyond classical A-T. There are several examples of genes in which different mutations lead to related but clinically different phenotypes. For example, different combinations of defective alleles of the ERCC2 gene may result in xeroderma pigmentosum (group D), Cockayne's syndrome or trichothiodystrophy—three diseases with different clinical features involving UV sensitivity (Broughton et al., 1994, 1995).

Different mutations in the CFTR gene may lead to full-fledged cystic fibrosis, or only to congenital bilateral absence of the vas deferens which is one feature of this disease (Chillon et al., 1995; Jarvi et al., 1995). A particularly interesting example is the X-linked WASP gene responsible for Wiskott Aldrich syndrome (WAS), characterized by immunodeficiency, eczema and thrombocytopenia. Most of the mutations responsible for this phenotype cause protein truncations; however, certain missense mutations may result in X-linked thrombocytopenia, which represents a partial WAS phenotype, while compound heterozygosity for a severe and mild mutation results in females in an intermediate phenotype (Kolluri et al., 1995; Derry et al., 1995).

In a similar manner, genotypic combinations of mutations with different severities create a continuous spectrum of phenotypic variation in many metabolic diseases.

Which phenotypes are most likely to be associated with milder ATM mutations? Since cerebellar damage is the early and severe manifestation of A-T, it is reasonable to assume that the cerebellum might also be affected to some extent in phenotypes associated with milder ATM mutations. Such phenotypes may include cerebellar ataxia, either isolated (Harding, 1993) or coupled with various degrees of immunodeficiency. The latter combination has indeed been described, sometimes with chromosomal instability, and is often designated “ataxia without telangiectasia” (Ying and Decoteau, 1983; Byrne et al., 1984; Aicardi et al., 1988; Maserati, 1988; Friedman and Weitberg, 1993). Friedman and Weitberg (1993) recently suggested a new clinical category of “ataxia with immune deficiency” that would include A-T as well as other cases of cerebellar degeneration with immune deficits. Evaluation of patients with cerebellar disorders with the present invention may reveal a higher frequency of such cases than previously estimated. However, in view of the pleiotropic nature of the ATM gene, the range of phenotypes associated with various ATM genotypes may be even broader, and include mild progressive conditions not always defined as clear clinical entities. Screening for mutations in this gene in such cases may reveal wider boundaries for the molecular pathology associated with the ATM gene.

### Example 4

### Determination of Genomic Organization

The exon/intron boundaries and the intron sizes were determined using long-distance PCR (Barnes, 1994; Cheng et al., 1994; Foord and Rose, 1994). Primers were designed based on the ATM cDNA sequence (Savitsky et al., 1995a,b) at 200-300 bp intervals. Templates for these reactions were cosmid and YAC clones, and human genomic DNA. PCR products were obtained in all cases, including those that span the largest intron, of 11 kb. In the large majority of cases, PCR products of the same size were obtained with all templates, and those obtained from genomic DNA were used for sequencing of the exon-intron junctions. Following initial reactions, new primers were designed as needed, based on the evolving knowledge of the gene structure. Exon-intron boundaries were determined at the sites where genomic and cDNA sequences diverged. Typical splice acceptor and donor sequences were found around these sites in all cases. During the search for the A-T gene, six exons were isolated by exon trapping (Shiloh et al., 1994b) using the vectors pSPL3 (Church et al., 1994) and éGET (Nehls et al., 1994a,b). Their boundaries coincided with those obtained by long-distance PCR.

The ATM gene is composed of 66 exons (FIG. 3 and Tables 1 and 5). The first two exons are alternatively spliced, and are designated 1a (SEQ ID No: 10) and 1b (SEQ ID No: 9). With the exception of the 3′ exon, ATM exons (SEQ ID Nos: 11-63) range in size from 64 to 372 bp, with an average of 149 bp. The introns vary considerably in size, from 100 bp to about 11 kb, with the majority in the range of 1-3 kb. The consensus dinucleotides GT and AG were found at the donor and acceptor splice sites of all introns, except for a variant donor site with a GC dinucleotide (reviewed in Jackson, 1991) present in the intron 3′ to exon 51. The first methionine of the open reading frame is located in exon 3, whereas the stop codon is located in the 3′ and largest exon of 3.6 kb. This exon includes a 3′ untranslated region (UTR)(SEQ ID No: 8) of about 3800 nucleotides.

The ATM gene contains one of the largest number of exons reported to date for a human gene. However, these exons are spread over a relatively compact genomic region of about 150 kb. The dystrophin gene, for example, consists of 79 exons spanning 2.4 Mb of genomic DNA (Roberts et al., 1993), while the Huntington's disease gene consists of 67 exons spread over 180 Kb (Ambrose et al., 1994b).

Throughout this application various publications and patents are referenced by citation or number. Full citations for the publications referenced are listed below. The disclosures of these publications in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
EXON-INTRON ORGANIZATION OF THE ATM GENE

| | | | | | | |
|---|---|---|---|---|---|---|
| | | Exon | Exon | Exon | | Size of |
| Exon | | First | length | Last | | 3′intron |
| No. | 5′intronic sequence | Base | (bp) | Base | 3′intronic sequence | (kb) |

| | | | | | | | | | |
|---|---|---|---|---|---|---|---|---|---|
| 1a§ | | −554 | AGGTAG | 120 | GCAGTG | −435 | gtaggggcgcggaggcaacgcagcggc | 0.18 | |
| | | | | | | | ttc | | |
| 1b§ | | −434 | TCCTCC | 316 | TACCAG | −119 | gtacagataagacaactacagtggatg | 1.10 | |
| | | | | | | | ata | | |
| 2 | ttttctattactgtgtttttgtttcct | −118 | AGGCAT | 88 | ATGAGG | −31 | gtaggatttgtatctgtttagttcatt | 2.70 | |
| | cag | | | | | | att | | |
| 3 | tatatatacctatatgtatttttttta | −30 | ACAGTG | 102 | CGAAAG | 72 | gtagtaaattacttaaattcaattttt | 0.08 | |
| | cag | | | | | | cct | | |
| 4 | aacccattattatttcctttttatttt | 73 | AAAGAA | 113 | TTTTAG | 185 | gtattctattcaaatttattttactgt | 1.30 | |
| | cag | | | | | | ctt | | |
| 5 | gagtttctgaaattgcattttgttttc | 186 | ATTTTT | 146 | ACAGAA | 331 | gtaagtgagttataaattataaataaa | 6.30 | |
| | ttgaag | | | | | | tggc | | |
| 6 | atgtttttctttatttgtttattttga | 332 | GAGCAC | 165 | GGTTAG | 496 | gtatgttttgaaggttgttgtttgtga | 8.10 | |
| | aatag | | | | | | attttt | | |
| 7 | catgactaataattttttttttttttt | 497 | AATTGT | 166 | TGCGAG | 662 | gtaatctaatctctttttcttttgttt | 0.67 | |
| | aag | | | | | | tgtattg | | |
| 8 | cccagttgagcttgtttgtttcttcac | 663 | ACAAGA | 239 | AAAAAG | 901 | gtataaaggaaatgtttactgttttga | 1.90 | |
| | ag | | | | | | attt | | |
| 9 | aaaaattacattttaattttttggatt | 902 | GTGCTT | 164 | CACCAG | 1065 | gtacagtaagtaggtcatgtcacattt | 1.70 | |
| | acag | | | | | | aga | | |
| 10 | gaaaaaagtggatttatttttatttta | 1066 | GTTTTT | 170 | GCCTTG | 1235 | gtaaagtgttaccattttctcattcag | 1.70 | |
| | cag | | | | | | tgt | | |
| 11 | ttccaaataaccctttttttttttttt | 1236 | GCTACA | 372 | TTCATG | 1607 | gtaagttcagcatgcattatcgtctga | 0.80 | |
| | tag | | | | | | ctt | | |
| 12 | ttttcacaattgtcctttgttttgtta | 1608 | TCCTGC | 195 | TCACAG | 1802 | gtaatttaagttcattagcatgctgct | 0.90 | |
| | tag | | | | | | gttt | | |
| 13 | ctaagtgaagctttttgtttttctttg | 1803 | TAATTT | 96 | AGAATG | 1898 | gtatgttatctaataatgctctttatc | 0.90 | |
| | tag | | | | | | att | | |
| 14 | ttatatattaaagatcttactttcttg | 1899 | TGAACA | 226 | TCTGAG | 2124 | gtgagattttttaaaaaaagaactaag | 2.20 | |
| | aag | | | | | | ctt | | |
| 15 | tatatatttttatttgtggtttacttt | 2125 | ATTACA | 126 | GCCAAC | 2250 | gtaggagaatttatactaataaagttt | 1.20 | |
| | aag | | | | | | cgg | | |
| 16 | gatttgcatttttccttctattcacaa | 2251 | TCTCTA | 126 | ACCAAG | 2376 | gtaagattttcttcttcttgttttgtt | 1.30 | |
| | tag | | | | | | ttgt | | |
| 17 | ttgcttggttctttgtttgtcttaatt | 2377 | AAGAGT | 90 | AGTTTA | 2466 | gtaagtatgcttcctgttttgctatca | 1.50 | |
| | gcag | | | | | | tat | | |
| 18 | cttgaacatctttgtttctcttccttg | 2467 | GCATCC | 172 | CCATAG | 2638 | gtaaatacatatttactacttgggatt | 1.10 | |
| | aag | | | | | | tct | | |
| 19 | ttagtgttaatgagtgctttttatttt | 2639 | GTGCCA | 200 | CATATG | 2838 | gtgagttacgttaaatgaagaagctct | 2.60 | |
| | tag | | | | | | tgg | | |
| 20 | cctgatttttttccctcctaccatctt | 2839 | TATCTA | 83 | ACTATC | 2921 | gtaagaaattaaaaccttatgttatgt | 0.10 | |
| | ag | | | | | | tca | | |
| 21 | aagttgaactttttttttttttttacc | 2922 | CAATGT | 156 | ATTTTG | 3077 | gtaggtacagtctatgttgtggtccta | 1.20 | |
| | acag | | | | | | ttt | | |
| 22 | tttaactttggaaaacttacttgattt | 1078 | GCATCT | 76 | CTTGAG | 3153 | gtgagtttttgcatttttttagtaaga | 0.10 | |
| | cag | | | | | | tct | | |
| 23 | tcatatttaaccacagttcttttcccg | 3154 | GCTGAT | 131 | CAATAG | 3284 | gtaatgggtcaaatattcatgaagtat | 7.00 | |
| | tag | | | | | | ttg | | |
| 24 | tttcattgtttctttccttcctgtctt | 3285 | ATTGTT | 118 | GAAATG | 3402 | gtaattttaagtaacatgtatttgctg | 1.30 | |
| | aag | | | | | | tta | | |
| 25 | ttacaatttttttttaaatttcttttt | 3403 | TCCCAT | 174 | AAAAAG | 3576 | gtatatatggatgagtattttattaga | 1.50 | |
| | aag | | | | | | agc | | |
| 26 | cttaacacattgactttttggttcgtg | 3577 | GTTTTA | 170 | CTATAG | 3746 | gtaagtttatacatgacatatgtgaaa | 1.30 | |
| | cag | | | | | | ttt | | |
| 27 | aacctgtattttaaatttttctatttt | 3747 | ATCTTG | 247 | AAACAG | 3993 | gtatggcttcaatttttatgtactttt | 3.00 | 3 .00 |
| | tag | | | | | | cat | | |
| 28 | taaatatattttaattttgtgcccttg | 3994 | ATTGAT | 116 | TTCAGG | 4209 | gtatgtacattttaaacttagagaact | 1.30 | |
| | cag | | | | | | agc | | |
| 29 | tgactgtattttttcccttaactctgt | 4110 | GGATTT | 127 | AGCCCT | 4236 | gtaagtatacatgatgagtttaataat | 0.52 | |
| | tag | | | | | | aga | | |
| 30 | aagtttttactaaatctgtttattttc | 4237 | GATTCC | 200 | CCAAAG | 4436 | gtaaataacatatttagaccaatatat | 3.00 | |
| | tag | | | | | | aag | | |
| 31 | ttgttgttgtttttttttctccctata | 4437 | GCCTTC | 175 | AAACAG | 4611 | gtaattttctgactcatcttcaaaatg | 0.53 | |
| | ttag | | | | | | gta | | |
| 32 | tataattttttctttttaaattatatt | 4612 | GTATTG | 165 | TTGGAG | 4776 | gtaataaaaatttcatcatctactatt | 1.50 | |
| | tag | | | | | | ttt | | |
| 33 | gttaaaagcaagttacattttctcttt | 4777 | GAAATT | 133 | CTCAGG | 4909 | gtgctaattttaaatgacatgggctat | 2.50 | |
| | tag | | | | | | tt | | |
| 34 | ttaaactaatttttaaaaaattatttc | 4910 | ATAATC | 96 | TTCTAG | 5005 | gtaaactacagtcatgcgctgcgtgac | 2.50 | |
| | tag | | | | | | attt | | |
| 35 | ctgaaatagaatttctatatgtag | 5006 | AGGCTG | 172 | AGATTG | 5177 | gtgagtatttattgataccttatatgt | 2.00 | |
| | | | | | | | aat | | |
| 36 | cttgataggcatttgaattgttttttt | 5179 | TGTCAA | 142 | AAAAAG | 5319 | gtctcttaagtaataaatgtttattga | 1.00 | |
| | cag | | | | | | ata | | |
| 37 | atttacattttctaatccctttctttc | 5320 | TTTTTA | 177 | TGTGAA | 5496 | gtaagaagattaattagtctgatataa | 1.60 | |
| | tag | | | | | | ttc | | |
| 38 | tattgggtggatttgtttgtatattct | 5497 | GTGAAA | 178 | ATTCAG | 5674 | gtattctattaaatttttaacattaat | 2.70 | |
| | ag | | | | | | act | | |
| 39 | ggactgaggggagatatttttgtttgt | 5675 | AGTCAG | 88 | AAAGAG | 5762 | gtaatgtaatgagtgttgcttcttacg | 2.00 | |
| | cag | | | | | | ttt | | |
| 40 | tgaatgacattatatctcatttttctt | 5763 | ACCTTC | 156 | GAAAAG | 5918 | gtaatggaatttagaatttttggtttt | 2.00 | |
| | tag | | | | | | taa | | |
| 41 | cattaaaagaggtgttcttgtgacaaa | 5919 | AAGTCT | 88 | TTACAG | 6006 | gtaaatattagaggctctattatttat | 3.30 | |
| | cag | | | | | | gac | | |
| 42 | cttcaatttttgttgtttccatgtttt | 6007 | GATCTT | 89 | TACTAG | 6095 | gtaaattgcatttttctaaacaacggt | 0.10 | |
| | cag | | | | | | atag | | |
| 43 | cccaaagctattttcacaatcttttct | 6096 | ACTACG | 103 | ATTCAG | 6198 | gtacattttttcccagatttggtaaag | 1.20 | |
| | tatag | | | | | | cca | | |
| 44 | aacttaaaaacaacaataactcctgtt | 6199 | GCCTTG | 149 | CGTCAG | 6347 | gtaagaaatttgacttgattttttttt | 2.50 | |
| | tag | | | | | | ttt | | |
| 45 | gtatatttttttctttgacttatctca | 6348 | CAAAGA | 105 | TGCCAG | 6452 | gtattatgaaaagacaaagttactgta | 1.40 | |
| | cag | | | | | | tttt | | |
| 46 | tttcagagtgtcttttcttttttcetc | 6453 | AGTAAA | 120 | CTCAAG | 6572 | gtatgtaattcgtatgacttggttatc | 4.00 | |
| | ctg | | | | | | cta | | |
| 47 | cttacatgaactctatgtcgtggcatt | 6573 | ATCAGT | 235 | ACTCAG | 6807 | gtaaatacaatttaaaactatgtcatc | 0.55 | |
| | cag | | | | | | tta | | |
| 48 | atttattcccatatgtcattttcattt | 6808 | CTCCCT | 268 | GCAGCG | 6975 | gtttgttttttttattggctggattag | 1.40 | |
| | cag | | | | | | tgt | | |
| 49 | tatattttaagattttgcctttcttat | 6976 | AACAAT | 114 | GAAAAG | 7089 | gtaagatttttggagcaacccttaaga | 1.30 | |
| | acag | | | | | | tag | | |
| 50 | tataatttaaattggttgtgttttctt | 7090 | GCAGTA | 218 | AAACAG | 7307 | gtaactaggtttctacaagtgacaatt | 1.00 | |
| | gaag | | | | | | tta | | |
| 51 | ttgtgttttaccttaattattctatgc | 7308 | ATACAC | 208 | ATGAAG | 7515 | gcaagtgttactcagcccaatattcta | 1.00 | |
| | aag | | | | | | ccc | | |
| 52 | cttaattttgtgtcttttttttaatgg | 7516 | AGAGAC | 114 | AATAAT | 7629 | gtaagtaaacctgaaaatcaaaccaca | 0.32 | |
| | tag | | | | | | ata | | |
| 53 | tgcataaatctaatagttcttttctta | 7630 | CTAATC | 159 | GATGAG | 7788 | gtattgggattaaccatacgtaccttc | 0.80 | |
| | cag | | | | | | tag | | |
| 54 | tatgaatgttttttgtttttttattaa | 7789 | GATCGA | 139 | AGAGAA | 7927 | gtatgttttttttaaagaagaaacgtt | 1.00 | |
| | tag | | | | | | act | | |
| 55 | tcactaaaatctcttcatttttaaata | 7928 | AAGGCA | 83 | ATTAAG | 8010 | gtaatttgcaattaactcttgattttt | 1.00 | |
| | cag | | | | | | ttt | | |
| 56 | ctattatcaatcatgtttatactttta | 8011 | GTGGAC | 141 | GTTAAG | 8151 | gtgagccttcccttctctggcttagcc | 0.80 | |
| | ttag | | | | | | ctt | | |
| 57 | acttgtttattcatgcttaattattct | 8152 | GGCCGT | 117 | TATAAG | 8268 | gtaactatttgtacttctgttagttca | 7.50 | |
| | gaag | | | | | | cca | | |
| 58 | aagtaaaaggtatttaatctgtaactc | 8269 | GTGGTT | 150 | ATGATG | 8418 | gtgagtgacacccaaaattaaaggtta | 2.20 | |
| | cag | | | | | | ttg | | |
| 59 | aaaataattatatatattctctattta | 8419 | GAGGTG | 166 | CTATTG | 8584 | gtaatcttcttgtacatatagtagatt | 1.50 | |
| | aag | | | | | | gag | | |
| 60 | tttcagattgtttgtttcttttttctc | 8585 | TTGGTT | 87 | ATCTAG | 8671 | gtaagtaataaaatctatgtatctatt | 6.00 | |
| | cag | | | | | | ctt | | |
| 61 | cctcctaacttcactgtattctttact | 8672 | GTGTTG | 115 | CAGAAG | 8786 | gtaagtgatatgaagtaaaggagggaa | 1.00 | |
| | ttag | | | | | | at | | |
| 62 | atccgtatttataatgtgttttgactc | 8787 | ATGCTG | 64 | GTAGAG | 8850 | gtctcggtcttttttggttgttttttt | 11.0 | |
| | tag | | | | | | ttttt | | |
| 63 | tacctaaaaacagatgtgctctctgta | 8851 | GTCCTT | 137 | TCTCAG | 8987 | gtgagcagtattttaagaaggtcctgt | 0.10 | |
| | tag | | | | | | tgt | | |
| 64 | tcactgaaacctttgtgtttttgtcct | 8988 | TGATAT | −3600 | | | | | |
| | tag | | | | | | | | |

*The first nucleotide of the open reading frame was designated +1.
§1a and 1b are alternatively spliced 5′non-coding exons.

TABLE 2
illustrates general mutation found in A-T patients.

| | | | Mutation | | | |
|---|---|---|---|---|---|---|
| | Ethnic/ | Complemen- | mPNA | | | |
| | geographic | tation | sequence | Protein | | Patient's |
| Patient¹ | origin | group⁴ | change | alteration | Codon⁹ | genotype¹⁰ |
| AT2RO | Arab | A | Deletion of 11 nt⁵ | Frameshift, truncation | 499 | Homozygote |
| AT3MO | Dutch | A | Deletion of 3 nt | Deletion, 1 residue⁸ | 1512 | Compound |
| | | | | | | heterozygote |
| AT15LA | Philippine | A | Insertion, +A | Frameshift, truncation | 557 | Compound |
| | | | | | | heterozyqote |
| AT3LA² | African-American | C | Deletion of 139 nt⁶/ | Frameshift, truncation | 1196 | Compound |
| AT4LA² | | | Deletion of 298 nt⁶ | | | heterozyqote |
| AT2BR | Celtic/Irish | C | Deletion, 9 nt | Deletion, 3 residues | 1198- | Homozygote |
| | | | | | 1200 | |
| AT1ABR | Australian | E | Deletion, 9 nt | Deletion, 3 residues | 1198- | Homozygote |
| AT2ABR | (Irish/British) | | | | 1200 | |
| AT5BI² | Indian/English | D | Deletion, 6 nt | Deletion, 2 residues | 1079- | Compound |
| AT6BI² | | | | | 1080 | heterozygotes |
| F-2079³ | Turkish | ND | Insertion, +C⁵ | Frameshift, truncation | 504 | Homozygote |
| AT29RM | Italian | ND | Deletion of 175 nt | Frameshift, truncation | 132 | Homozyqote |
| AT103LO | Canadian | ND | Insertion, +A | Frameshift, truncation | 1635 | Homozyqote |
| F-596³ | Palestinian Arab | ND | Deletion⁷ | Truncation | Most | Homozygote |
| | | | | | of ORF | |

¹Cell line designation.
²Sibling patients in both of whom the same mutation was identified.
³Patient expected to be homozygous by descent for an A-T mutation.
⁴According to the methods of Jasper. et al. (1988) ND: not determined.
⁵An identical sequence change was observed in genomic DNA
⁶No evidence for deletion was observed in genomic DNA. In both siblings, a normal mRNA was observed in addition to the two deleted species. The two deleted mRNAs may represent abnormal splicing events caused by a splice site mutation.
⁷Reflects a genomic deletion segregating with the disease in Family N.
⁸The deleted serine residue is located within the PI3-kinase signature sequence (1507-1527 of SEQ ID No:2).
⁹Numbers refer to residue positions in SEQ ID No:2.
¹⁰In all the compound heterozygotes, the second mutation is still unidentified.

TABLE 3
Mutations in the ATM gene in patients with classical A-T.

| | | | | | |
|---|---|---|---|---|---|
| mRNA | predicted | | | Ethnic/ | |
| sequence | protein | | | geographical | |
| change¹ | alteration | Codon⁸ | Patient | origin | Genotype¹¹ |

Truncations and exon skipping delations:

| | | | | | |
|---|---|---|---|---|---|
| 9001delAG | Truncation | 3001 | 91RD90⁹ | Turkish | Hmz |
| 8946insA | Truncation | 2983 | AT103LO | American | Hmz |
| 8307G→A | Trp→ter; | 2769 | AT2SF | American | Compd Htz |
| | truncation | | | | |
| 8283delTC | Truncation | 2762 | AT28RM | Italian | Compd Htz |
| 8269del403² | Truncation | 2758 | AT12RM | Italian | Hmz |
| 8269del1503 | Del, 50 aa | 2758 | F-2086 | Turkish | Compd Htz |
| | | | GM9587 | American | Compd Htz |
| 8140C→T | Gln→ter; | 2714 | IARC12/AT3 | French | Hmz |
| | truncation | | | | |
| 7883del5 | Truncation | 2628 | ATF104 | Japanese | Hmz |
| | | | JCRB16 | Japanese | Compd Htz |
| 7789del139/7630del298^{4,5} | Truncation | 2544 | AT4LA | Carribean Black | Comp Htz |
| 7630del159³ | Del, 53 aa | 2544 | F-2086 | Turkish | Compd Htz |
| | | | AT13BER | | Compd Htz |
| 7517del4 | Truncation | 2506 | AT43RM¹⁰ | Italian | Hmz |
| | | | AT59RM¹⁰ | Italian | Hmz |
| | | | AT22RM¹⁰ | Italian | Hmz |
| | | | AT57RM¹⁰ | Italian | Compd Htz |
| | | | AT7RM¹⁰ | Italian | Compd Htz |
| | | | AT8RM¹⁰ | Italian | Compd Htz |
| 6573del5 | Truncation | 2192 | AT12ABR | Australian | Compd Htz |
| 6348del105³ | Del. 35 aa | 2116 | IARC15/AT4 | French | Hmz |
| 6199del149³ | Truncation | 2067 | WG1101 | Canadian | Hmz |
| 5979del5 | Truncation | 1994 | AT5RM | Italian | Compd Htz |
| 5712insA | Truncation | 1905 | AT15LA | Philippino | Compd Htz |
| 5554insC | Truncation | 1852 | F-2079⁹ | Turkish | Hmz |
| 5539del11 | Truncation | 1847 | AT2RO⁹ | Arab | Hmz |
| 5320del355⁴ | Truncation | 1774 | AT7RM | Italian | Compd Htz |
| 5320del7 | Truncation | 1774 | AT2SF | American | Compd Htz |
| 5178del142³ | Truncation | 1727 | AT50RM | Italian | Compd Htz |
| 4612del165³ | Del, 55 aa | 1538 | ATL105 | Japanese | Hmz |
| 44437del175³ | Truncation | 1480 | AT29RM | Italian | Hmz |
| 4110del127³ | Truncation | 1371 | AT2TAN⁹ | Turkish | Hmz |
| 3403del174³ | Del, 58 aa | 1135 | F-2095 | Turkish | Compd Htz |
| 2839del33³ | Truncation | 947 | F-2080⁹ | Turkish | Hmz |
| | | | AT10TAN⁹ | Turkish | Hmz |
| 2467del372^{3,8} | Del. 124 aa | 823 | AT6LA | English/Irish | Hmz |
| 2377del90³ | Del, 30 aa | 793 | AT21RM⁹ | Italian | Hmz |
| 22284delCT | Truncation | 762 | F-169⁹ | Palestinian Arab | Hmz |
| 2125del125³ | Truncation | 709 | F-2078⁹ | Turkish | Hmz |
| 2113delT | Truncation | 705 | AT5RM | Italian | Compd Htz |
| 1563delAG⁵ | Truncation | 522 | AT8LA⁹ | Swiss/German | Hmz |
| 1339C→T | Arg→ter; | 447 | F-2005⁹ | Druze | Hmz |
| | truncation | | | | |
| 1240C→T | Gln→ter; | 414 | AT26RM | Italian | Hmz |
| | truncation | | | | |
| 755delGT | Truncation | 252 | AT24RM | Italian | Hmz |
| 497del7514⁷ | Truncation | 166 | F-596⁹ | Palestinian-Arab | Hmz |
| −30del215 | Incorrect | 5′ UTR | F-303 | Bedouine | Hmz |
| | initiation | | | | |
| 537C→T | Truncation | 35 | F-320 | Moroccan Jewish | Hmz |
| | | | F-191 | Arab | Hmz |
| | | | F-2084 | Bedouin | Hmz |
| 7240del4 | Truncation | 2414 | L-111 | Bedouin | Hmz |
| 2125del125 | Truncation | 709 | AT58RM | Italian | Hmz |
| 1407del201 | Truncation | 469 | AT44RM | Italian | Hmz |
| 4777del830 | Truncation | 1593 | F-2024 | Arab | Hmz |
| 9140C→T | Arg→ter: | 3047 | AT35RM | Italian | Hmz |
| | truncation | | | | |
| 3403del174 | Del, 58aa | 1135 | AT42RM | Italian | Hmz |
| | | | A(−T)2RM | Italian | Hmz |
| 4612del165 | Del, 55 aa | 1538 | AT25LA | American | Hmz |

In-frame genomic deletions and insertions:

| | | | | | |
|---|---|---|---|---|---|
| 8578del3 | Del, 1 aa | 2860 | AT3NG | Dutch | Compd Htz |
| 7636del9 | Del, 3 aa | 2547 | AT2BR | Celtic/Irish | Hmz |
| | | | AT1ABR | Australian (Irish) | Hmz |
| | | | AT1SF | American | Compd Htz |
| 7278del⁶ | Del, 2 aa | 2427 | AT5BI | Indian/English | Compd Htz |
| | | | GM5823 | English | Compd Htz |
| 5319ina9 | Ins, 3 aa | 1174 | 251075-008T | Finnish | Compd Htz |
| 5435del3 | Del, 1 aa | 1812 | AT53RM | Italian | Hmz |

Other base substitutions:

| | | | | | |
|---|---|---|---|---|---|
| 9170G→C | ter→Ser | ter | F-2059⁹ | Turkish | Hmz |
| | Extension of protein | | | | |
| | by 29 amino acids | | | | |
| 8711A→G | Glu2904Gly | 2904 | AT41RM | Italian | Hmz |
| 2T→C | Met→Thr | 1 | AT8BI | British | Compd Htz |
| | Initiation codon | | | | |
| | abolished | | | | |
| 129T→C | Leu→Pro | 43 | AT51RM | Italian | Comp Htz |

¹Presented according to the nomenclature proposed by Beaudet & Tsui (1993). Nucleotids numbers refer to their positions in the sequence of the ATM transcript (accession number U33841). The first nucleotide of the open reading trace was designated +1.
²Three adjacent exons skipped.
³One exon skipped.
⁴This allele produces two transcripts, with one or two ajacent exons skipped.
⁵The same mutation was found in two affected siblings.
⁶Two exons skipped.
⁷This transcript is produced by an allele containing a large genomic deletion spanning approximately 85 Kb within the ATM gene in Family ISAT 9 (Savitsky, et al., 1995a).
⁸For deletions, the number of the first codon on the amino terminus side is indicated. Codon numbers are according to the ATM protein sequence published by Savitsky et al. (1995b). In each section of the table, the mutations are ordered according to the codon numbers in this column, beginning with the one closest to the carboxyl terminus.
⁹Consanguineous family.
¹⁰All patients are from the same region.
¹¹Genotypic combinations in which the mutation was found. Hmz: homozygote; Compd Htz: compound heterozygote. Each patient represents one family.

TABLE 4
Comparison of the ATM protein to related
proteins in different species

| | Size | | % identity/similarity | |
|---|---|---|---|---|
| Protein | (aa) | Species | Carboxy terminus* | Rest of proteins** |
| TEL1 | 2789 | S. cerevisiae | 45/67 | 19/44 |
| MEC1 | 2368 | S. cerevisiae | 37/63 | 20/46 |
| rad3 | 2386 | S. pombe | 38/59 | 21/46 |
| MEI-41 | 2356 | D. melangaster | 37/59 | 22/47 |
| TOR1 | 2470 | S. cerevisiae | 33/58 | 19/45 |
| TOR2 | 2473 | S. cerevisiae | 35/60 | 20/45 |
| mTOR | 2549 | R. norvegicus | 32/59 | 18/44 |
| DNA- | 4096 | H. sapiens | 28/51 | 18/43 |
| PK_{cs} | | | | |

*350 aa of the carboxy terminus, containing the P1-3 kinase-like domain.
**The entire protein excluding the carboxy terminal 350 aa. An average value is given, since the values obtained for different parts of the proteins vary only by 1-3%.

TABLE 5

GENOMIC ORGANIZATION

| | |
|---|---|
| Exon 3 | (SEQ ID No:11) |

| | |
|---|---|
| 1 | AGGCATACAT CACAATTTGG AATTATGCAT TGGTTTATCA ATTTACTTGT |
| 51 | TTATTGTCAC CCTGCTGCCC AGATATGACT TCATGAGGGT AGGATTTGTA |
| 101 | TCTGTTTAGT TCATTATTTG TCTAGCTATA AGTAGTAAAT ATTGTTTGCA |
| 151 | ACTATCAGTG AATGAGCATC TTCTGTTTAT GTAGATAATA CTGAACTG |

************************************************************

| | |
|---|---|
| Exons 4-5 | (SEQ ID No:12) |

| | |
|---|---|
| 1 | TTACAGCATT ACTTGTATAG ATTTTAAGGA GATCTCATTT TAAATACGGA |
| 51 | AATGTTAAGA AAAATTATTG TGCCTTTGAC CAGAATGTGC CTCTAATTGT |
| 101 | ACAGATAAAT CTAACTATAA ATGCTGCAGT ATAAAATAAT TACATACACA |
| 151 | TTTTTTCACA CCTCTTTCTC TCTATATATG CATATATACA TACACATATA |
| 201 | TATACCTATA TGTATTTTTT TTACAGACAG TGATGTGTGT TCTGAAATTG |
| 251 | TGAACCATGA GTCTAGTACT TAATGATCTG CTTATCTGCT GCCGTCAACT |
| 301 | AGAACATGAT AGAGCTACAG AACGAAAGGT AGTAAATTAC TTAAATTCAA |
| 351 | TTTTTCCTTG AAATGTGTGA TTAGTAACCC ATTATTATTT CCTTTTTATT |
| 401 | TTCAGAAAGA AGTTGAGAAA TTTAAGCGCC TGATTCGAGA TCCTGAAACA |
| 451 | ATTAAACATC TAGATCGGCA TTCAGATTCC AAACAAGGAA AATATTTGAA |
| 501 | TTGGGATGCT GTTTTTAGGT ATTCTATTCA AATTTATTTT ACTGTCTTTA |
| 551 | TTTTTCTCTT TCATATTTAT TTCTGTTGTG ATATTACTTT TGTGTGTAAG |
| 601 | TCTTAACATT TATCTTTGAT TCCTATATAT CATTATGCCT TGCATATGAA |
| 651 | TTTGGCATTT AATATTTATC CAAAACATAA TTTTTAAAGG TTGTTCATAT |
| 701 | AGAAACTTAA AAATTATAAA TTATTTCTTC AATAAAATGT TTTAGACATA |

************************************************************

| | |
|---|---|
| Exon 6 | (SEQ ID No:13) |

| | |
|---|---|
| 351 | GCAGTAAAGC AATAGAAAGT CATAGAAGAT TAAGAGCTTT GCAGACCAGA |
| 401 | TATTAAATTG GTCTTGTAGG AGTTAGGCCT TGAAAGAGAG ATTTAATTGT |
| 451 | TTTATTTGTT TTTTTCAGCT GATGTAGTAA TCTAAGCAAG GTGGTTTAAA |
| 501 | AGTTGCTCTT TGTGATGGCA TGAACAGCTT TTGAAATTAT TATAATTTAA |
| 551 | GTATTCAACG AGTTTCTGAA ATTGCATTTT GTTTTCTTGA AGATTTTTAC |
| 601 | AGAAATATAT TCAGAAAGAA ACAGAATGTC TGAGAATAGC AAAACCAAAT |
| 651 | GTATCAGCCT CAACACAAGC CTCCAGGCAG AAAAAGATGC AGGAAATCAG |
| 701 | TAGTTTGGTC AAATACTTCA TCAAATGTGC AAACAGAA |

************************************************************

| | |
|---|---|
| Exon 7 | (SEQ ID No:14) |

| | |
|---|---|
| 1 | GAGCACCTAG GCTAAAATGT CAAGAACTCT TAAATTATAT CATGGATACA |
| 51 | GTGAAAGATT CATCTAATGG TGCTATTTAC GGAGCTGATT GTAGCAACAT |
| 101 | ACTACTCAAA GACATTCTTT CTGTGAGAAA ATACTGGTGT GAAATATCTC |
| 151 | AGCAACAGTG GTTAGGTATG TTTTGAAGGT TGTTGTTTGT GAATTTTTCC |
| 201 | TCATGAAATG AAACTTCACC AAAGAAAGCA CTCTGTCTGT ATCTGTCTAT |
| 251 | ATCCCCCAAG TGACCTGACA GGTTTAACAG TACTTTAGTA AAATTATATG |
| 301 | GTTATCGAAC TGACCCTTAA TTTTTATTTA TTATGTAGCT TTTGAATAA |

************************************************************

| | |
|---|---|
| Exon 8 | (SEQ ID No:15) |

| | |
|---|---|
| 1 | AATTGTTCTC TGTGTACTTC AGGCTCTATC TGAAACCTTC ACAAGATGTT |
| 51 | CATAGAGTTT TAGTGGCTAG AATAATTCAT GCTGTTACCA AAGGATGCTG |
| 101 | TTCTCAGACT GACGGATTAA ATTCCAAATT TTTGGACTTT TTTTCCAAGG |
| 151 | CTATTCAGTG TGCGAGGTAA TCTAATCTCT TTTTCTTTGT TTTGTATTGA |
| 201 | AATACTTTTG ATCTTGCAAG ACCATGTTTT AGACTCAGTA ACTAAAAATT |
| 251 | CTACCTTAAA ATAAAACATT GATCCATCAT AACAGAACTA GTGGATTCCT |
| 301 | AAAGAGACAA CCAAGTCCAA CACTTTCTGA ATATCCAATA TGCAGAACAC |
| 351 | TACGTGAAGT TTTCAAGGGG GAGATGTGTC TTGCTGAT |

************************************************************

| | |
|---|---|
| Exon 9 | (SEQ ID No:16) |

| | |
|---|---|
| 1 | ACAGAGTGGT CTCTTACACC AAATAAGAAC TAATTTTTTG TCAGTGTGAA |
| 51 | GTAATGCTGT GATTTTTTTT TTAATGAATA GTTTTGAAAT TAAGACTACT |
| 101 | GTTTGAAAAT TAGGGTTTTG TTTTTTTTTC TTTCAGCATA CCACTTCATA |
| 151 | ACTGTTCAGT TTGTACAGTT TGTTCCCCCT GTTATACCCA GTTGAGCTTG |
| 201 | TTTGTTTCTT CACAGACAAG AAAAGAGCTC TTCAGGTCTA AATCATATCT |
| 251 | TAGCAGCTCT TACTATCTTC CTCAAGACTT TGGCTGTCAA CTTTCGAATT |
| 301 | CGAGTGTGTG AATTAGGAGA TGAAATTCTT CCCACTTTGC TTTATATTTG |
| 351 | GACTCAACAT AGGCTTAATG ATTCTTTAAA AGAAGTCATT ATTGAATTAT |
| 401 | TTCAACTGCA AATTTATATC CATCATCCGA AAGGAGCCAA AACCCAAGAA |
| 451 | AAAGGTATAA AGGAAATGTT TACTGTTTTG AATTTGCTTC TTCATTCAAA |
| 501 | CATAGAAGTC TAAGTATAAA ATTAGTGTTC TTTAGGAGGA TATGACTTTC |
| 551 | CTCTGGATTT CTCTGGTTGA TAATGTTACT TAGCCATGAG AATGTTTTTC |
| 601 | ATAGAGTTTT T |

************************************************************

| | |
|---|---|
| Exon 10 | (SEQ ID No:17) |

| | |
|---|---|
| 1 | GTGCTTATGA ATCAACAAAA TGGAGAAGTA TTTTATACAA CTTATATGAT |
| 51 | CTGCTAGTGA ATGAGATAAG TCATATAGGA AGTAGAGGAA AGTATTCTTC |
| 101 | AGGATTTCGT AATATTGCCG TCAAAGAAAA TTTGATTGAA TTGATGGCAG |
| 151 | ATATCTGTCA CCAGGTACAG TAAGTAGGTC ATGTCACATT TAGAAATTTC |
| 201 | CTGTTAATTT TTTTTTTAAA CTGGGCATTT GGGCTTTTAA AACCTGTGTT |
| 251 | CTCACAAAAA GCCTATAAAA TGACTCTGTA CATGCAACTA TTCCTTTCAA |
| 301 | ACTATCAGAA ATATTTGGAA TTACCCTTTT AACTTAAAAG TTAATGCTTT |
| 351 | TGCAGATATT TGAAAACTAA CAATGAACTT TTTCATTCTT AAATGATTGT |
| 401 | CTCTAGGAAA TAAGGTGACC CTAACCCTAA TGATTCGATT CGACTCGA |

************************************************************

| | |
|---|---|
| Exon 11 | (SEQ ID No:18) |

| | |
|---|---|
| 1 | GGTTGTGGTT ATACGAGATC GTGCTGTTCC ACTCCAACCT GGGCAACAAC |
| 51 | AGCGAAATCT GGCTCAAAAA AAAAAAAAAA GAAAAAAGTG GATTTATTTT |
| 101 | TATTTTACAG GTTTTTAATG AAGATACCAG ATCCTTGGAG ATTTCTCAAT |
| 151 | CTTACACTAC TACACAAAGA GAATCTAGTG ATTACAGTGT CCCTTGCAAA |
| 201 | AGGAAGAAAA TAGAACTAGG CTGGGAAGTA ATAAAAGATC ACCTTCAGAA |
| 251 | GTCACAGAAT GATTTTGATC TTGTGCCTTGGTAAAGTGTT ACCATTTTCT |
| 301 | CATTCAGTGT CATTTTAATC TCTTGTATGT TATTTTTCAG AAAACTTTCA |
| 351 | GTGGAATCCT TTCATCTCAA CCAGAACTAA GTCATTTGTC TACCCCCAAA |
| 401 | CCTATTACTA GCAAAGGGAT ATGTGATTGC CATGACAAAT GAGATCAATC |
| 451 | ATTAATGGCT CATTTGCTTG GGCCAAGTGC AGGGCCACCT ATTTTAATCA |

************************************************************

| | |
|---|---|
| Exon 12 | (SEQ ID No:19) |

| | |
|---|---|
| 1 | AACTATTAAC AGCCAGTTTA TTTTTAGAGT ACTATGGAAA TGATGGTGAT |
| 51 | TTCTAATTAG GATATTGTAA GAGTACCATG TCTATATATT TCCTTTTAGT |
| 101 | TTGTTAATGT GATGGAATAG TTTTCAAATA TCCTTTTTTT TTTTTTTTTA |
| 151 | GGCTACAGAT TGCAACCCAA TTAATATCAA AGTATCCTGC AAGTTTACCT |
| 201 | AACTGTGAGC TGTCTCCATT ACTGATGATA CTATCTCAGC TTCTACCCCA |
| 251 | ACAGCGACAT GGGGAACGTA CACCATATGT GTTACGATGC CTTACGGAAG |
| 301 | TTGCATTGTG TCAAGACAAG AGGTCAAACC TAGAAAGCTC ACAAAAGTCA |
| 351 | GATTTATTAA AACTCTGGAA TAAAATTTGG TGTATTACCT TTCGTGGTAT |
| 401 | AAGTTCTGAG CAAATACAAG CTGAAAACTT TGGCTTACTT GGAGCCATAA |
| 451 | TTCAGGGTAG TTTAGTTGAG GTTGACAGAG AATTCTGGAA GTTATTTACT |
| 501 | GGGTCAGCCT GCAGACCTTC ATGGTAAGTT CAGCATGCAT TATGTCTGAC |
| 551 | TTACAGATAA ACACACACAG ACACACACAC ACTCACATAT CCCTGATCAT |
| 601 | TTCCATAGTT TGTTACTTCA GTTAAAGATG TCAAATTCTA TTTCAGATGC |
| 651 | TTTTCTTGTT TGGCCGAGAA GACTTAATAA ATGCATAAGT GAATTTAGTT |
| 701 | TCAAATGTTG ACAAATTATT AAAGACTAAT GTTAAGGAAT TTCTTTTT |

************************************************************

| | |
|---|---|
| Exon 13 | (SEQ ID No:20) |

| | |
|---|---|
| 1 | AGAATTATGA AGAGTTTAAA TTTCTTTTAT GTGCAATTTA TCATTATTTA |
| 51 | TTAAATAGCC ATGTTTAAAT TGTAGTACTA TGCACTGTTA ATAAACGAGC |
| 101 | TATTTTTTAA TCAAGAATCT TCCCAAATGT AATCAGACTT TTAACAGTTT |
| 151 | TTATGTTCAT TTAGTCACCT TAACTAAATG TATGTGCCAG GCACTGTCCT |
| 201 | GATAGATAAA GTCTTTGCCC CTCCAATAGC TTGCTTTTCA CAATTGTCCT |
| 251 | TTGTTTTGTT ATAGTCCTGC AGTATGCTGT TTGACTTTGG CACTGACCAC |
| 301 | CAGTATAGTT CCAGGAACGG TAAAAATGGG AATAGAGCAA AATATGTGTG |
| 351 | AAGTAAATAG AAGCTTTTCT TTAAAGGAAT CAATAATGAA ATGGCTCTTA |
| 401 | TTCTATCAGT TAGAGGGTGA CTTAGAAAAT AGCACAGAAG TGCCTCCAAT |
| 451 | TCTTCACAGG TAATTTAAGT TCATTAGCAT GCTGCTGTTT TTTTTGTTTG |
| 501 | TTTTATCAGG CTCTCTCCAC TTATTTGATG CCAGATGGCT TTATTTTATA |
| 551 | ATAATAATGC AGAATTTCCC AGATCTAACC TTAATTATTA AATATTATGT |
| 601 | TTGTTTTTAC AGtTATCTGT GTCTTTATGC CTGATTGCTT CTGAAATAAA |
| 651 | GGGTTGTCTC ACTGTGAGAA TATGGGGGAT GTGCATGAAA AATGCACAT |

************************************************************

| | |
|---|---|
| Exon 14 | (SEQ ID No:21) |

| | |
|---|---|
| 1 | TGGATCCAAT TAAAGTACTT TTGCTTTAAT TTTACAACCT TTTATTTATT |
| 51 | TCAGAAATAA TGTTAAACAT GCTGTTTCTA AACAGTATTG GAAATGATAA |
| 101 | TAACAATGGT TGTCCTCCTT AAATTGTCCT TTTAGATATT AAGAAATTTA |
| 151 | GTATAGATGA AAGCAATTTT AATCTAGGAT CCAAATTTTA GAAGTCAAGA |
| 201 | TTTATAGCTA AACATGGATG TTAAAGTTTA AAGTATTCTT TACATGGCTT |
| 251 | TTGGTCTTCT AAGTGAAGCT TTTTGTTTTT CTTTGTAGTA ATTTTCCTCA |
| 301 | TCTTGTACTG GAGAAAATTC TTGTGAGTCT CACTATGAAA AACTGTAAAG |
| 351 | CTGCAATGAA TTTTTTCCAA AGCGTGCCAG AATGGTATGT TATCTAATAA |
| 401 | TGCTCTTTAT CATTTTAAGC TATAGCTTTA ATTACAAAGA TGATAATTTT |
| 451 | CGCTGGGTAG tAGCTGCATC TTAATAANGG TCACCTAACT TGGTCCAAAA |
| 501 | AAATTGCAAC TGTTAGCCAG GGAAGAGGTT GTTTTAATTC AGTGATTGT |

************************************************************

| | |
|---|---|
| Exon 15 | (SEQ ID No:22) |

| | |
|---|---|
| 1 | TTTCTACTGA ATAATGACAT TTGATATAAG TAGGTCTCAA AGTCCGAAGA |
| 51 | AGAGAAGGCA TTTAAAAGAA TAATCTATTA ATTATATGAA GTAGTCTTTG |
| 101 | AATGATGTAG ATACTAGGTT AATGTTTTCC TTTGTAATAT ATTGCTAATA |
| 151 | CATATAAGGC AAAGCATTAG GTACTTGGTT TATATATTAA AGATCTTACT |
| 201 | TTCTTGAAGT GAACACCACC AAAAAGATAA AGAAGAACTT TCATTCTCAG |
| 251 | AAGTAGAAGA ACTATTTCTT CAGACAACTT TTGACAAGAT GGACTTTTTA |
| 301 | ACCATTGTGA GAGAATGTGG TATAGAAAAG CACCAGTCCA GTATTGGCTT |
| 351 | CTCTGTCCAC CAGAATCTCA AGGAATCACT GGATCGCTGT CTTCTGGGAT |
| 401 | TATCAGAACA GCTTCTGAAT AATTACTCAT CTGAGGTGAG ATTTTTTAAA |
| 451 | AAAAGAACTA AGCTTATATA TGATTCAACT TTGGTAAACT GTTAGGAAGG |
| 501 | AGAAATAGGG GCAGGAAAAA CAGCAAGGAT GGTGGGAGGC TTCATTTTAA |
| 551 | AAGCAAAGTG GCAGTAAAGG GCTCTAAATT GGACAACTTA GCATAATTAA |
| 601 | AGGAAAACTC AAGAATAATA ATTTGAGTAC TTCCTTT |

************************************************************

| | |
|---|---|
| Exon 16 | (SEQ ID No:23) |

| | |
|---|---|
| 1 | CCATCAGGAG ATACTTAGGC TATTTTTCTT GAGAATCCTG GTTATAATTC |
| 51 | TACAGTGATC TCCTAGTTGT TTTTAGAGCT ATCCAGGATA TGCCACCTTT |
| 101 | AACTCAGTTA ACTGAACTTT TGTTTTTTAA TATGTATGTA GAATTTGTTC |
| 151 | TTACAAAAGA TAGAGTATAC TAAATTATTT ATGAAATATA TATATTTTTA |
| 201 | TTTGTGGTTT ACTTTAAGAT TACAAATTCA GAAACTCTTG TCCGGTGTTC |
| 251 | ACGTCTTTTG GTGGGTGTCC TTGGCTGCTA CTGTTACATG GGTGTAATAG |
| 301 | CTGAAGAGGA AGCATATAAG TCAGAATTAT TCCAGAAAGC CAAGGTAGGA |
| 351 | GAATTTATAC TAATAAAGTT TCGGATAAAT TTGAATGAAA TGTATTCCTG |
| 401 | TGAAAATTAT TACATTTGTT TGGAAGACAT TAAATTGTAT GCAGGTTAAC |
| 451 | CCTTTCTCTT TTATTTATGT AATGTGAGAA GAAATTATAC TATGTATTTT |
| 501 | TTAAATTGTT TTAATTGTTT AATTTTTAAT TATTATTATA CTTTAAGTTC |
| 551 | TGGGGTACAT GATGCAGA |

************************************************************

| | |
|---|---|
| Exon 17 | (SEQ ID No:24) |

| | |
|---|---|
| 1 | AAGATTTGCC ATTTTAAAAA ATTGTTAATG AGTTTTGCTT AAACTGTATG |
| 51 | ACCACGTGGA ACTTCTAAAA ACATTTCATT TTTTCTCTTA AGTGCACTTT |
| 101 | ATTTTTTATT TTATAGTATG TCCAAGATCA AAGTACACTG TAAAAAGCAA |
| 151 | TACTAAACTA TAATTTTAAC TGGAATTTGC ATTTTTCCTT CTATTCACAA |
| 201 | TAGTCTCTAA TGCAATGTGC AGGAGAAAGT ATCACTCTGT TTAAAAATAA |
| 251 | GACAAATGAG GAATTCAGAA TTGGTTCCTT GAGAAATATG ATGCAGCTAT |
| 301 | GTACACGTTG CTTGAGCAAC TGTACCAAGG TAAGATTTTC TTCTTCTTGT |
| 351 | TTTGTTTTTT GAGATAGGAT CTTTCTCTGT CACCCAGGCT GGAGTGCAGT |
| 401 | GGGATTGTCA CAACTCATTG TAGCCTTGAC CTCCTGGTTT CCAGCAATTC |
| 451 | TCCTGCCTCA GTCTCCCAAG TAATTGGGAC TACAGGCATG TACCACCTAG |
| 501 | CTAAAATTTT CTTTTTACTT GAAAGTGTAG C |

************************************************************

| | |
|---|---|
| Exon 18 | (SEQ ID No:25) |

| | |
|---|---|
| 1 | TGTTGTTAAA GCACAATGAA AGATGTACAG TATAGTTATT ATAACTCTAA |
| 51 | GAAAAGATGT GTTTTTGAAG CAGCATATAT ATTGGCCCTA ATAGTAAACT |
| 101 | ATTTATCTAC ATTCCATTCA AGATAGAGAA AACACTGTCT GCCAAGAATA |
| 151 | ATTGTTTTTA TTTCTTTGTT GCTTGGTTCT TTGTTTGTCT TAATTGCAGA |
| 201 | AGAGTCCAAA TAAGATTGCA TCTGGCTTTT TCCTGCGATT GTTAACATCA |
| 251 | AAGCTAATGA ATGACATTGC AGATATTTGT AAAAGTTTAG TAAGTATGCT |
| 301 | TCCTGTTTTG CTATCATATT TTGATTCTAA TAGGCATAAT TTTTTTGTTG |
| 351 | AAATATCTTT GTAAATAAGG ATGCATCTCA CAACATATAG CTCTTAACAT |
| 401 | TTTTACAAAT GTGGAAATTA AGGCCAGGTG CGGTGGCTCA TGCCTGTAAC |
| 451 | CCCAGCACTT TGGGAGGCCG AGGTGG |

************************************************************

| | |
|---|---|
| Exon 19 | (SEQ ID No:26) |

| | |
|---|---|
| 1 | TTACAGGTGC CCACCACCAC ACCCAGCTAA TTTTTGTATT TTAAATAGAG |
| 51 | ACAAGGTTTC ACCATGTTGG CCAGGCTGGT TTCGAACTCC CGACCTCAGG |
| 101 | TGATCCACCT GGCTCAGCCT CCCAAATTGC TGAGATTACA GATGTGAGCC |
| 151 | ACTGTGCCCA GCCTGATTAG GTAAATTTTG ACTACAGCAT GCTCCTGCAA |
| 201 | GAAGCCATCT TGAACATCTT TGTTTCTCTT CCTTGAAGGC ATCCTTCATC |
| 251 | AAAAAGCCAT TTGACCGTGG AGAAGTAGAA TCAATGGAAG ATGATACTAA |
| 301 | TGGAAATCTA ATGGAGGTGG AGGATCAGTC ATCCATGAAT CTATTTAACG |
| 351 | ATTACCCTGA TAGTAGTGTT AGTGATGCAA ACGAACCTGG AGAGAGCCAA |
| 401 | AGTACCATAGGTAAATACAT ATTTACTACT TGGGATTTCT TTTACTTCTT |
| 451 | TATATTGATT TGGCAGTATA AGAGGCCTCA TTGATATCAA TTTTGTGCTT |
| 501 | ATTTCATTTT CTCTTAGTAT AGCCTTTTAG GATTGTTCCT TTCTTATATA |
| 551 | CTTTATTTTT TTTTTATTTT TACTGGAATT TATTAGTTTC ATATTTTATC |
| 601 | CTCCATAGAA GGAACTTAAG ATAACTATTA AAGA |

************************************************************

| | |
|---|---|
| Exon 20 | (SEQ ID No:27) |

| | |
|---|---|
| 1 | TTTAAATTAT TTCTTGACAA CAGAATCTTG GATAATTTTT CAAAAAGACT |
| 51 | TTTGAAGCTT TCAGTATATA ATTAATTTCA CTATAATTTT GCTTTTCATA |
| 101 | TACTTTTTTT TGTGAAGAGG AGGAAATTTG AGTTAATATG ACTATATATG |
| 151 | GCTGTTGTGC CCTTCTCTTA GTGTTAATGA GTGCTTTTTA TTTTTAGGTG |
| 201 | CCATTAATCC TTTAGCTGAA GAATATCTGT CAAAGCAAGA TCTACTTTTC |
| 251 | TTAGACATGC TCAAGTTCTT GTGTTTGTGT GTAACTACTG CTCAGACCAA |
| 301 | TACTGTGTCC TTTAGGGCAG CTGATATTCG GAGGAAATTG TTAATGTTAA |
| 351 | TTGATTCTAG CACGCTAGAA CCTACCAAAT CCCTCCACCT GCATATGGTG |
| 401 | AGTTACGTTA AATGAAGAAG CTCTTGGATT TTATCTGATG TTGCTGACTA |
| 451 | AATGTAATGA GTTGACATGT AAGAATCACA TGGTGTCTTT GAAGAATTGA |
| 501 | AATTGCTTTC TTGAGAAATG AACCTGAGAC TAGTTGGAAA ATAACACTTT |
| 551 | TAACGTGCTG TGAGCAAATT TAAGTGGATG CTGAAATATT AAA |

************************************************************

| | |
|---|---|
| Exons 21-22 | (SEQ ID No:28) |

| | |
|---|---|
| 1 | AGGCGCCTGC CACCACCCCT GGCTAATTTT TGTGTTTTTA GTAGAGATGG |
| 51 | AGTTTCACCA TATTGGCCAG GCTGTTCTCA AACTCCTGAC CTTGTGATCT |
| 101 | GCCTGCTTCA GCCTCCCAAA GTGCTGGGAT TACAGGTtTG AGCCACTGCA |
| 151 | CCCGGCCTAT GTTTATATAC TTTTTAAAGT AAATGATTTG TGGATAAACC |
| 201 | TGATTTTTTT CCCTCCTACC ATCTTAGTAT CTAATGCTTT TAAAGGAGCT |
| 251 | TCCTGGAGAA GAGTACCCCT TGCCAATGGA AGATGTTCTT GAACTTCTGA |
| 301 | AACCACTATCGTAAGAAATT AAAACCCTTA TGTTATGTTC ACTTTAAAGT |
| 351 | TATAAAATAA CTGATGTGTT CTTAAGCTTA ATAAAGTGGA ACTTTTTTTT |
| 401 | TTTTTTTACC ACAGCAATGT GTGTTCTTTG TATCGTCGTG ACCAAGATGT |
| 451 | TTGTAAAACT ATTTTAAACC ATGTCCTTCA TGTAGTGAAA AACCTAGGTC |
| 501 | AAAGCAATAT GGACTCTGAG AACACAAGGG ATGCTCAAGG ACAGTTTCTT |
| 551 | ACAGTAATTG GAGCATTTTGGTAGGTACAG TCTATTTTGT GGTCCTATTT |
| 601 | TTCTTTTGCT ATCTGTGGAT ACGAATGCAA GTTTTGTATC CACATCAGTG |
| 651 | ATTTCTTCTG ATCTGCCTAC ATAGCTAATA CATCTGGAAA GAATAGCAGA |
| 701 | ATGTTATTTG TGTTTCCCTC AGTCGCTTGA AGAACTACAT TGCTTTTTGT |
| 751 | TTAAGGCTTG GCTTTCTAA |

************************************************************

| | |
|---|---|
| Exons 23-24 | (SEQ ID No:29) |

| | |
|---|---|
| 1 | ATTCCTTTTA CCACTAATTT CCTTTTAGCT TGAATTTTTG GCAAGGTGAG |
| 51 | TATGTTGGCA TATTCCACAT AATGACAAAT AAGTTTAGCA CAGAAAGACA |
| 101 | TATTGGAAGT AACTTATAAT AACCTTTCAG TGAGTTTTCT GAGTGCTTTT |
| 151 | ATCAGAATGA TTATTTAACT TTGGAAAACT TACTTGATTT CAGGCATCTA |
| 201 | ACAAAGAGA GGAAATATAT ATTCTCTGTA AGAATGGCCC TAGTAAATTG |
| 251 | CCTTAAAACT TTGCTTGAGG TGAGTTTTTG CATTTTTTTA GTAAGATCTC |
| 301 | CATTGAAAAT TTTAAAGCAG TCTTTGTTTG TTAATGAGTA ATTTTTCTCT |
| 351 | ATTTCATATT TAACCACAGT TCTTTTCCCG TAGGCTGATC CTTATTCAAA |
| 401 | ATGGGCCATT CTTAATGTAA TGGGAAAAGA CTTTCCTGTA AATGAAGTAT |
| 451 | TTACACAATT TCTTGCTGAC AATCATCACC AAGTTCGCAT GTTGGCTGCA |
| 501 | GAGTCAATCA ATAGGTAATG GGTCAAATAT TCATGAAGTA TTTGGAATGC |
| 551 | TGCAGATGGC AGTAGAATGT CTTACATAGT AACAGCTCAC AGTTGCAATA |
| 601 | TTAAAAATAG CTAACACTTG TTGAGTATAT ACGGTGTGCC TGGCATTTAT |
| 651 | GTTTATTCTT AATTCTTATA CTTCTGTCAC TTAGATTCTA TTATTTCCTT |
| 701 | CAATTTATAA AT |

************************************************************

| | |
|---|---|
| Exon 25 | (SEQ ID No:30) |

| | |
|---|---|
| 1 | CTAAGCTGCT GGTCTGAACC TCTTTAAATA AACTCAGGTT TTGTTAGTCT |
| 51 | TTAAGAAAGA GCTAGTATGT TATTATGTCT CACAGAGTGA TTTATTTTTG |
| 101 | TTCTGGATA TGCTTTGGAA AGTAGGGTTT GAAATTAGAA AATTATTTCA |
| 151 | CTTTTTGTTT GTTTGTTTGC TTGCTTGTTT TAAGATTGTT CCAGGACACG |
| 201 | AAGGGAGATT CTTCCAGGTT ACTGAAAGCA CTTCCTTTGA AGCTTCAGCA |
| 251 | AACAGCTTTT GAAAATGCAT ACTTGAAAGC TCAGGAAGGA ATGAGAGAAA |
| 301 | TGGTAATTTT AAGTAACATG TATTTGCTGT TATCATATGC TTGCTATGAA |
| 351 | TATCCCATAA ATTACTTCAC CAAGTTTGGT ATAAGAGAGT TTATAATCCA |
| 401 | GTAGTTTACA GTATAAAGCT GCTCTTCCCC AACTGTATGA ATTGATTGAA |
| 451 | ACTGCATTCT TTCTGGGTCA CAATGGGTCA AATCATAGCA ATTTCTTTTG |
| 501 | GTTTAGCA |

************************************************************

| | |
|---|---|
| Exon 26 | (SEQ ID No:31) |

| | |
|---|---|
| 1 | AAAATTGGTA TGTAAACAAT TACAATTTAC ATTACAATTT TTTTTTAAAT |
| 51 | TTCTTTTTAA GTCCCATAGT GCTGAGAACC CTGAAACTTT GGATGAAATT |
| 101 | TATAATAGAA AATCTGTTTT ACTGACGTTG ATAGCTGTGG TTTTATCCTG |
| 151 | TAGCCCTATC TGCGAAAAAC AGGCTTTGTT TGCCCTGTGT AAATCTGTGA |
| 201 | AAGAGAATGG ATTAGAACCT CACCTTGTGA AAAAGGTATA TATGGATGAG |
| 251 | TATTTTATTA GAAGCTTCCT TAGGTCACTG TGAAATAATT TAAAAAGTTA |
| 301 | AAGCTAGATT TTCTGAGTGG CACTTATTTA AGACTAGGAA ACAATTTTAT |
| 351 | TTTTTAGGTT GGGAATATTG GAAAGCAGTT ATACAAAAAC TATTCAAATG |
| 401 | GTATATTTAT GGTATGCACT GTTTCTTACA TTCCA |

************************************************************

| | |
|---|---|
| Exon 27 | (SEQ ID No:32) |

| | |
|---|---|
| 1 | TGCTATGtGT CAGATACTGT GCCAGTTGAG TACATTTTCT TAATTATTAT |
| 51 | TCCCATCTCA TAGATGAGGA AATCAAGAAA AGTTGAATGA ATGTTGTTTC |
| 101 | TAGGTCCTAC TCTAAATAAT ATTAACAAGC ATTTAAATGA TTTATTTTTT |
| 151 | TCATTTTTCT TAACACATTG ACTTTTTGGT TCGTGCAGGT TTTAGAGAAA |
| 201 | GTTTCTGAAA CTTTTGGATA TAGACGTTTA GAAGACTTTA TGGCATCTCA |
| 251 | TTTAGATTAT CTGGTTTTGG AATGGCTAAA TCTTCAAGAT ACTGAATACA |
| 301 | ACTTATCTTC TTTTCCTTTT ATTTTATTAA ACTACACAAA TATTGAGGAT |
| 351 | TTCTATAGGT AAGTTTATAC ATGACATATG TGAAATTTGT TTAATTTAAA |
| 401 | ATTAGTTAAC AATACTTAGC AAGTCCCCTC ACCAGCAACA CACATACCAT |
| 451 | ACCCATACAC ATGTGTGTGT GGGAGCCTAC ATAGTATGAG AAGCAGGACA |
| 501 | GCTTCTTTTA ATAAGAATGT ATTGAAGGGA GTCACTGGAC TTCAGATC |

************************************************************

| | |
|---|---|
| Exon 28 | (SEQ ID No:33) |

| | |
|---|---|
| 1 | ATTTTTTTAA TGTGACTATT TAGAATTTAC TTAATTTTTC CATTTATAAA |
| 51 | ATTAAAGAAT GTTTAATAAT CTGGATAAAG TATGATACTT TAATGCTGAT |
| 101 | GGTATTAAAA CAGTTTTTAA GAACTATTTT ATAAAATTTT ACTTGGAAAA |
| 151 | GTTATATATA ACCTGTATTT TAAATTTTTC TATTTTTAGA TCTTGTTATA |
| 201 | AGGTTTTGAT TCCACATCTG GTGATTAGAA GTCATTTTGA TGAGGTGAAG |
| 251 | TCCATTGCTA ATCAGATTCA AGAGGACTGG AAAAGTCTTC TAACAGACTG |
| 301 | CTTTCCAAAG ATTCTTGTAA ATATTCTTCC TTATTTTGCC TATGAGGGTA |
| 351 | CCAGAGACAG TGGGATGGCA CAGCAAAGAG AGACTGCTAC CAAGGTCTAT |
| 401 | GATATGCTTA AAAGTGAAAA CTTATTGGGA AAACAGGTAT GGCTTCAATT |
| 451 | TTTATGTACT TTTCATTCCC TGAATGATAT GAGATATAAC CTTTAAGTTT |
| 501 | TAAGGCTATT TATTCGATTT ATTCGTATTT ATATATTGAA ACTTAGCTTG |
| 551 | TGGTAATCAT TATCTAGCAT AGCCAACCCA TGAATTTTTT TGGTTATGTC |
| 601 | GTGTTGTCTC CCTCTGATTG GCTTTTAACT A |

************************************************************

| | |
|---|---|
| Exon 29 | (SEQ ID No:34) |

| | |
|---|---|
| 1 | GGGGGCCTTG TTTGGCTGAT TTTCATACTT TTTCCTCTCA GTCTACAGGT |
| 51 | TGGCTGCATA GAAGAAAAAG GTAGAGTTAT TTATAATCTT GTAAATCTTG |
| 101 | GACTTTGAGT CATCTATTTT CTTTTACAGT CATCGAATAC TTTTGGAAAT |
| 151 | AAGGTAATAT ATGCCTTTTG AGCTGTCTTG ACGTTCACAG ATATAAAATA |
| 201 | TTAAATATAT TTTAATTTTG TGCCCTTGCA GATTGATCAC TTATTCATTA |
| 251 | GTAATTTACC AGAGATTGTG GTGGAGTTAT TGATGACGTT ACATGAGCCA |
| 301 | GCAAATTCTA GTGCCAGTCA GAGCACTGAC CTCTGTGACT TTTCAGGGTA |
| 351 | TGTACATTTT AAACTTAGAG AACTAGCTCT AACTTCACAA GTTTTTAAAG |
| 401 | AAGTTTATTG GTTGACACCT TCAATGTCTA TTTCAATTTA TAGACATCAC |
| 451 | TCTTTTTAAA AAATTTTCTT CAAAAATAGC CACCTTTGAA TTGAGGTAA |

************************************************************

| | |
|---|---|
| Exons 30-31 | (SEQ ID No:35) |

| | |
|---|---|
| 1 | TAATCTGATT TATATATCTG GACTGTGATA TGTCATTTGT GATTTTATTG |
| 51 | AAAGTATAGT TTTTCAGTAG AAAAATGGTT TTTGAATTTG GGGGTTATTA |
| 101 | AAATCTAAAT TTTCATTTTG GAAGTTCACT GGCTATGAAC AAAACTTTTT |
| 151 | AAAACGATGA CTGTATTTTT TCCCTTAACT CTGTTAGGGA TTTGGATCCT |
| 201 | GCTCCTAATC CACCTCATTT TCCATCGCAT GTGATTAAAG CAACATTTGC |
| 251 | CTATATCAGC AATTGTCATA AAACCAAGTT AAAAAGCATT TTAGAAATTC |
| 301 | TTTCCAAAAG CCCTGTAAGT ATACATGATG AGTTTAATAA TAGAACATTC |
| 351 | CTTCTTTTTT AGCTAAAAAA ACTTTGTAAA TACATCTTAA AGAGGAAAAG |
| 401 | TAAACAAATG AAAAATTTAT CTCATAATTA AAAAGGAAAA CATTCATTTA |
| 451 | CAAGTTTAAA TGGTATTTTA CTTGTCAGCA TTAATTGAAA TATGTTACAT |
| 501 | ATGAGAACAG AATCTTGTGA CACTTTAGTG ATATATTAGC TCAGGGAATA |
| 551 | TATCTACTTT TTCATAGGAA TATACTATTT AATTGTAGTT TACTTTCTGA |
| 601 | AAATTAAATA AATTGGCAAT AGTTTAAGAT AGTAATTTTC TTAATGTAAC |
| 651 | ATTTTGTACT TGATATCAAA CCCAAATCTA AATTCTGTTA TTTAGTTATT |
| 701 | TTAAATATAA AATGTGTAGG TATTCAAATA TTTGAAGAAA AAATATAAAG |
| 751 | TGTATTTATT GTAGCCGAGT ATCTAATTAA ACAAGTTTTT ACTAAATCTG |
| 801 | TTTATTTTCT TAGGATTCCT ATCAGAAAAT TCTTCTTGCC ATATGTGAGC |
| 851 | AAGCAGCTGA AACAAATAAT GTTTATAAGA AGCACAGAAT TCTTAAAATA |
| 901 | TATCACCTGT TTGTTAGTTT ATTACTGAAA GATATAAAAA GTGGCTTAGG |
| 951 | AGGAGCTTGG GCCTTTGTTC TTCGAGACGT TATTTATACT TTGATTCACT |
| 1001 | ATATCAACCA AAGGTAAATA ACATATTTAG ACCAATATAT AAGCAGTCTT |
| 1051 | TCTATCCTGT TCTTCCTGTT TTTTTGCTTT GTTTTGTTTT GTTTTGAGAC |
| 1101 | AAAGACTCAC TCTGTCCGCC CAGGCTGGGT GCAGTCACGG CTCACTGCAT |
| 1151 | CCTCAACCTC CTGGGTTCAG ATTATCCTTC CTCCTCACCC TGCCGGGTAG |
| 1201 | CTGGGGCTAC AGGAG |

************************************************************

| | |
|---|---|
| Exons 32-33 | (SEQ ID No:36) |

| | |
|---|---|
| 1 | ATGTTTCTAT TAAAGGATGG AAGCTTAGAG CTGCCTATTC TGCATTTTGC |
| 51 | TGATGTGACT TCTCTTTTTG GCTTATAAGC CATTAAAATA TTTTNGTCAA |
| 101 | GGCATATAAG AATTAGAGAT GCTGAACCAA AGGACTTCTG AATGAATTTA |
| 151 | TTTCAGAGTA ATTTTCCAGA ACTTACTGGT TGTTGTTGTT TTTTTTTCTC |
| 201 | CCTATATTAG GCCTTCTTGT ATCATGGATG TGTCATTACG TAGCTTCTCC |
| 251 | CTTTGTTGTG ACTTATTAAG TCAGGTTTGC CAGACAGCCG TGACTTACTG |
| 301 | TAAGGATGCT CTAGAAAACC ATCTTCATGT TATTGTTGGT ACACTTATAC |
| 351 | CCCTTGTGTA TGAGCAGGTG GAGGTTCAGA AACAGGTAAT TTTCTGACTC |
| 401 | ATCTTCAAAA ATGGTATTTA AAATATATAA AGTATTGTTA GAAGGATTTG |
| 451 | AGTGTTTTAT GTTTATTTGG TATAATTGGT GATTTTATTG AGAATATTTT |
| 501 | TTGTAAAATG ATTGGAAAAA TATTCTTAAT GAATTAACCT TTGTAATCAA |
| 551 | TTACAGAGCA CTTGGTACTT TTGATAGTTT TATCTACTGT GCTGAAGTGG |
| 601 | AGAGGTAGTC AAAACTAGGG ATAGCAGTTC GCAACGTTAT GGTGGTATTT |
| 651 | GAGTTACTAC TTATATAAAC TGTTTCATTA ATATTGGCAT TTTTTTTAAC |
| 701 | CTCAGTACCC ATCTTGTAGT AGTACCTTAC ATAGTTATTG AATTATTTGA |
| 751 | AAACACAGAA ACTAAAAGCT GGGTATCTTA GACGTAATAA GAACATTTAA |
| 801 | TCTGATCTAG GTTAATAGAT TTTATCATTT ATTACAGTAA GTTTTGTTGG |
| 851 | CTTACTTTAA AATTATTTCT CTCCTTATAA TTTTTTCTTT TTAAATTATA |
| 901 | TTTAGGTATT GGACTTGTTG AAATACTTAG TGATAGATAA CAAGGATAAT |
| 951 | GAAAACCTCT ATATCACGAT TAAGCTTTTA GATCCTTTTC CTGACCATGT |
| 1001 | TGTTTTTAAG GATTTGCGTA TTACTCAGCA AAAAATCAAA TACAGTAGAG |
| 1051 | GACCCTTTTC ACTCTTGGAGGTAATAAAAA TTTCATCATC TACTATTTTT |
| 1101 | TATTAGAGAA CATAGTAGTA CTTTTCAAAA ATCTGTAATG CTCTAGCAGT |
| 1151 | AAAAAATGGA ATCTTTTCTT TAATTGTGAT TAAAAATATA TACGTAGGCC |
| 1201 | AGGCACATTG GCTCATGCCT ATAATCTCAG CACTTT |

************************************************************

| | |
|---|---|
| Exon 34 | (SEQ ID No:37) |

| | |
|---|---|
| 51 | AAATCATTTA TTTCTTCCTT GATTAGTAGT AATAGAGACA TGAGTCAGTG |
| 101 | TCTATAAATG GCACTTAACT AATTTTTTTC TTTTATTAAG TTTTATTTCA |
| 151 | CAGGCTTAAC CAATACGTGT TAAAAGCAAG TTACATTTTC TCTTTTAGGA |
| 201 | AATTAACCAT TTTCTCTCAG TAAGTGTTTA TGATGCACTT CCATTGACAA |
| 251 | GACTTGAAGG ACTAAAGGAT CTTCGAAGAC AACTGGAACT ACATAAAGAT |
| 301 | CAGATGGTGG ACATTATGAG AGCTTCTCAG GGTGCTAATT TTAAATGACA |
| 351 | TGGGCTATTT CTACCTGTTT CTTTTTGGAA AGAATATTTT GCAAAGTCTT |
| 401 | GCTCTTGGTT TCATTGTCAC AGACTTAGTT CAGACTCTCA TCATTTAGTT |
| 451 | CAGACCCTCA TTTCTCATCT AACTGTAAAA CTGGTCCTAA CTGGTCTTCT |
| 501 | CACCCTGAAC TCTTCCTGTT TTATTCATCC TCTGCCAGAT G |

************************************************************

| | |
|---|---|
| Exon 36 | (SEQ ID No:38) |

| | |
|---|---|
| 1 | ACCTCTGTCT CCCAAAGTGC TGGGATTACA GTCGTGAGCC ACCGCACTCG |
| 51 | GCCTTAAGGT TAATTCTTGA AGTACAGAAA AACAGCATTA TAGTTTGGAA |
| 101 | ATTAGAAAAT ATCAGTTTTA TGTATGATCT CTTACCTATG ACTCTACTGA |
| 151 | AATAGAATTT CTATATGTAG AGGCTGTTGG AAGCTGCTTG GGAGAAGTGG |
| 201 | GTCCTATAGA TTTCTCTACC ATAGCTATAC AACATAGTAA AGATGCATCT |
| 251 | TATACCAAGG CCCTTAAGTT ATTTGAAGAT AAAGAACTTC AGTGGACCTT |
| 301 | CATAATGCTG ACCTACCTGA ATAACACACT GGTAGAAGAT TGGTGAGTAT |
| 351 | TTATTGATAC CTTATATGTA ATCTCAATAT GACATTCATG GAGAATGATA |
| 401 | CTTCACACAA ATAGATATTC TCAGTAACTA AAGCTTTGTC CTTTTTTAAA |
| 451 | TCTCAGTGTC TTTATGAAAA TTCTTATATT TTTATTAATT CACATAATTA |
| 501 | TTTACCCTAC TATGTGCCAG ACACTTGATA TAATGGT |

************************************************************

| | |
|---|---|
| Exon 37 | (SEQ ID No:39) |

| | |
|---|---|
| 1 | GTTGCAGTGA TTAGTAATTC AAGTTTACTG AATGACTAGT GAAAGTCCTT |
| 51 | TGATACTTTT ATTTGATATT GGAGAATTTT GTAAATGTAA AGTTTCCTAA |
| 101 | AACCAATTTT AAATTTTAGT TTTGAAATTT TTTCAGTGGA GGTTAACATT |
| 151 | CATCAAGATT AATAACTGGT GTACTTGATA GGCATTTGAA TTGTTTTTTT |
| 201 | CAGTGTCAAA GTTCGATCAG CAGCTGTTAC CTGTTTGAAA AACATTTTAG |
| 251 | CCACAAAGAC TGGACATAGT TTCTGGGAGA TTTATAAGAT GACAACAGAT |
| 301 | CCAATGCTGG CCTATCTACA GCCTTTTAGA ACATCAAGAA AAAAGGTCTC |
| 351 | TTAAGTAATA AATGTTTATT GAATACCCAG CATATCTAAA ACAGTTCTGT |
| 401 | TTGCTGTGGG TCATGACTGT TAAATTGCTT GAAATAGTAT TGTACTAACT |
| 451 | ATTAACCTTT CCTATAAGTA ATTTAAGCCA TATTTCATAA ATCCAGGGAA |
| 501 | TGTGTTATTT TTAATTTATT ATGGCAGTGT G |

************************************************************

| | |
|---|---|
| Exon 38 | (SEQ ID No:40) |

| | |
|---|---|
| 1 | AAACATAAAT GTTTTCATCT TAAAAGGTAA ACATTGCCTC CAGATTTAGT |
| 51 | TTTAACTGTA TTTAGCTTTA TTCAGAAAGA TTTGTTATAC TCATTTTGTG |
| 101 | TAGGAAAGGT ACAATGATTT CCACTTCTCT TATTTACATT TTCTAATCCC |
| 151 | TTTCTTTCTA GTTTTTAGAA GTACCCAGAT TTGACAAAGA AAACCCTTTT |
| 201 | GAAGGCCTGG ATGATATAAA TCTGTGGATT CCTCTAAGTG AAAATCATGA |
| 251 | CATTTGGATA AAGACACTGA CTTGTGCTTT TTTGGACAGT GGAGGCACAA |
| 301 | AATGTGAAAT TCTTCAATTA TTAAAGCCAA TGTGTGAAGT AAGAAGATTA |
| 351 | ATTAGTCTGA TATAATTCCT TGTTTATGAC CTGTTTATCT AAAGAGTGCT |
| 401 | GTGATACTGC ACATCATCTT CACATAATAT CACCCCCACT CAAACTGTTG |
| 451 | TAAATTTATT AAAGTGAGCA TCCGTATTTA GTCATAACTT TATGCATTAG |
| 501 | GTTTCAGCTT CGGGATAGCA ACATACT |

************************************************************

| | |
|---|---|
| Exon 39 | (SEQ ID No:41) |

| | |
|---|---|
| 1 | CCTAGTGTGG TTTTTTAAAC ACCACCTAAT ACATGTTTTT TGTTTGTTTT |
| 51 | TTTAGCAGTA TGTTGAGTTT ATGGCAGATT AATCTATCAT CTTTTAGAAA |
| 101 | TTTAATATGT CAACGGGGCA TGAAAATTTT AAGTAAAATG TATTAATTTT |
| 151 | ACTCATTTTT ACTCAAACTA TTGGGTGGAT TTGTTTGTAT ATTCTAGGTG |
| 201 | AAAACTGACT TTTGTCAGAC TGTACTTCCA TACTTGATTC ATGATATTTT |
| 251 | ACTCCAAGAT ACAAATGAAT CATGGAGAAA TCTGCTTTCT ACACATGTTC |
| 301 | AGGGATTTTT CACCAGCTGT CTTCGACACT TCTCGCAAAC GAGCCGATCC |
| 351 | ACAACCCCTG CAAACTTGGA TTCAGGTATT CTATTAAATT TTTAACATTA |
| 401 | ATACTGTAAA CTCAGTTCTA GAGAAAGATG GATTTAAGAT GGAATCCCAC |
| 451 | TAAAAGCACT TTACAGGATT AAATCTATAA CCTCTAAATT TGTTTCTTCA |
| 501 | TCTATGGAAT GGAGATAAAA GTTGCCAACA GTTGCAACAA GTTTTCAATG |
| 551 | AAATAATGTG TGTAAAG |

************************************************************

| | |
|---|---|
| Exon 40 | (SEQ ID No:42) |

| | |
|---|---|
| 1 | TAAGTTCTCA CTTTTTTTAA GATAACAGTT TCTTTTAAAA GCAAAAGAAA |
| 51 | TCCTATTAAA TTCCTTCAGA ACCAATTTTG TGTTAGGTAC TGCCCACCAG |
| 101 | AACCTTATAG CATAGTGGGA GACAGACACA TAAACAAGAA GGAAGAAGGT |
| 151 | GTGTAAGCAA GAATGCCTGG GACTGAGGGG AGATATTTTT GTTTGTCAGA |
| 201 | GTCAGAGCAC TTTTTCCGAT GCTGTTTGGA TAAAAAATCA CAAAGAACAA |
| 251 | TGCTTGCTGT TGTGGACTAC ATGAGAAGAC AAAAGAGGTA ATGTAATGAG |
| 301 | TGTTGCTTCT TACGTTTAGG ATCTAGAGTG TAACTTGTTA ACTATCGGCT |
| 351 | GAATTTTAAC ATGATTATTT TAGGTGAAGG TGTTGCAAAG TGTTATATTT |
| 401 | AATTTGTGTG ATATTTATAT CTCCTTGCAG tAATCCATAT TCAGGATAGC |
| 451 | AGTTTGGTTA AATCAGTGTC AAGAA |

************************************************************

| | |
|---|---|
| Exon 41 | (SEQ ID No:43) |

| | |
|---|---|
| 1 | TTAATTAAAT AGGACTCTTC AGCCATGTTA TCTTATAATG TTTATAGGTA |
| 51 | TATATTGGGG AAATGTGGTT TTTGGGAATT TGTAATTTTC TGTTAAGCAG |
| 101 | TCACTACCAT TGTATTCTAT ATCAACATGC TTTTATTTTG ATATTGAAGT |
| 151 | TTAAAAAAGT GAATGACATT ATATCTCATT TTTCTTTAGA CCTTCTTCAG |
| 201 | GAACAATTTT TAATGATGCT TTCTGGCTGG ATTTAAATTA TCTAGAAGTT |
| 251 | GCCAAGGTAG CTCAGTCTTG TGCTGCTCAC TTTACAGCTT TACTCTATGC |
| 301 | AGAAATCTAT GCAGATAAGA AAAGTATGGA TGATCAAGAG AAAAGGTAAT |
| 351 | GGAATTTAGA ATTTTTGGTT TTTAAAATTA ATGTTGGCAT TGTCTCAATA |
| 401 | AGGGTATATA GTAAAGATTT ATTTTGCCTC CTGTTCCCCA TTTAAAAGAT |
| 451 | ATTTTAGATA GAAATTTTGT TTTAAAGTGA AATTATAATA AATTTTTAAA |
| 501 | AAGGAATATG TAATTCCTGT TCTGAAAT |

************************************************************

| | |
|---|---|
| Exon 42 | (SEQ ID No:44) |

| | |
|---|---|
| 1 | CTTTTCCATC CTAGGTATAA ATGGTATTAT GTTTTAAAGT ATAAGTGATT |
| 51 | TATTCTGTTT TGTTTGCCAC CTTCATTAGT TTTTTTCTGT CAAAGTCTAT |
| 101 | AGTATATGTA TTCAGGAGCT TCCAAATAGT ATGTTCTCAT TAAAAGAGGT |
| 151 | GTTCTTGTGA CAAACAGAAG TCTTGCATTT GAAGAAGGAA GCCAGAGTAC |
| 201 | AACTATTTCT AGCTTGAGTG AAAAAAGTAA AGAAGAAACT GGAATAAGTT |
| 251 | TACAGGTAAA TATTAGAGGC TCTATTATTT ATGACAGTAT TTATCTCATA |
| 301 | CTTTGGGTTA TTTTGTTATA GACACTGTAC AGATGCCATG TGATTTTTAA |
| 351 | ACTGAATTTA CTTACTGGAC TAAGCATCAT ATATATAAAA TTATGGTCTG |
| 401 | AAGCTTAAGC CTTAGAGTAG ACAGACTTGA GTTCTAATAC TGAC |

************************************************************

| | |
|---|---|
| Exons 43-44 | (SEQ ID No:45) |

| | |
|---|---|
| 1 | TTGTCCTGCA CAGTTCAAAC TCGTGTTGTT TGAACTGTAT TTCAGAACTG |
| 51 | TATTTCAGAA TCATTACATT TTATTTCTAT AACATAACAT TTAGAGTTGG |
| 101 | GAGTTACATA TTGGTAATGA TACAATTTAA AATTTGCTAA ATTTATAGAC |
| 151 | CGATTTTTTT TCCTTCTTCA ATTTTTGTTG TTTCCATGTT TTCAGGATCT |
| 201 | TCTCTTAGAA ATCTACAGAA GTATAGGGGA GCCAGATAGT TTGTATGGCT |
| 251 | GTGGTGGAGG GAAGATGTTA CAACCCATTA CTAGGTAAAT TGCATTTTTC |
| 301 | TAAACAACGG TATAGTAATT CTGTTTATGA AGGAGTTATG TGTGTGTTAA |
| 351 | ACCCAAAGCT ATTTTCACAA TCTTTTCTTA TAGACTACGA ACATATGAAC |
| 401 | ACGAAGCAAT GTGGGGCAAA GCCCTAGTAA CATATGACCT CGAAACAGCA |
| 451 | ATCCCCTCAT CAACACGCCA GGCAGGAATC ATTCAGGTAC ATTTTTTCCC |
| 501 | AGATTTGGTA AAGCCATCAC TAGTGTAGTG CTGAGGTTAT TTCAGTATGT |
| 551 | TGGTGGATAT TTACACAGCC AGATAAACTC TAGAGATAAG ACTAGAACTT |
| 601 | ATCTGTTTTT CAGAGGATTA GGCTAAACAT TCAGGGATAC TCCTGAAGCA |
| 651 | GAGGGATGCA AAAAAAAGAG AAAAAATTCA GGGAGACA |

************************************************************

| | |
|---|---|
| Exon 45 | (SEQ ID No:46) |

| | |
|---|---|
| 1 | CCTCCTGCCT CAGCCTCCCA AAGTGCTGAG ATTACAGGCA TGAGCCACCA |
| 51 | CACCCAGCTG ATATTTTGGG ATTTTAAATG ATATTGTGAA CTAAAATTTG |
| 101 | TCTAAGTTAA TTTGTATCTT TGCTGTTTTT TTCTCTGGTT TTCTGTTGAT |
| 151 | ATCTTTGATT ACTTAACTTA AAAACAAAAT AACTCCTGTT TAGGCCTTGC |
| 201 | AGAATTTGGG ACTCTGCCAT ATTCTTTCCG TCTATTTAAA AGGATTGGAT |
| 251 | TATGAAAATA AAGACTGGTG TCCTGAACTA GAAGAACTTC ATTACCAAGC |
| 301 | AGCATGGAGG AATATGCAGT GGGACCATTG CACTTCCGTC AGGTAAGAAA |
| 351 | TTTGACTTGA TTTTTTTTTT TTTGCCTCTC TCCTCATTCT AAACAACAAC |
| 401 | TGTTTTTCTC TTCTATGA |

************************************************************

| | |
|---|---|
| Exon 46 | (SEQ ID No:47) |

| | |
|---|---|
| 1 | TTTTTCTGCT TAAAGAATTT AAATGACTCA TAAAATTTGT ATTTCTTACC |
| 51 | AAAAATTCTA GAAATGCATT TTTTAGAATG GAGAAATGTT AATTTAAAAA |
| 101 | TTTTGTCCTT TGGTGAAGCT ATTTATACAT GTATATCTTA GGGTTCTGTT |
| 151 | TTTAAGTATA TTTTTTTCTT TGACTTATCT CACAGCAAAG AAGTAGAAGG |
| 201 | AACCAGTTAC CATGAATCAT TGTACAATGC TCTACAATCT CTAAGAGACA |
| 251 | GAGAATTCTC TACATTTTAT GAAAGTCTCA AATATGCCAGGTATTATGAA |
| 301 | AAGACAAAGT TACTGTATTT TAACATTTAA TGTCATGGCT TCTTTTCTGA |
| 351 | AAACTTGAGA AACAATTTTA ATGTAAGGAT TTGCATTGAT GAAGAGATAA |
| 401 | AGACTTGGTG GCTGTGATCA GATGTTTCCT TGTAATTCTC TGCCCTCCTT |
| 451 | CAAAACAAAT TGTTTCTGGG ATTCCAGGTT CATTCTTTAC CCTGACCCTT |
| 501 | CAAGAAAGTT TTG |

************************************************************

| | |
|---|---|
| Exon 47 | (SEQ ID No:48) |

| | |
|---|---|
| 1 | AGTGTAAGTG GACCATGCAT TTAAATTTGT GTTGTTCAAG GGTCAGTTGT |
| 51 | ATTCTGTTTC CACTGCTATT TTGTACTCAC TGCTGCTTGT TAGTATTATT |
| 101 | AGATCAGTAG CATAGCCTAT GATGAGAACT CTTTAACAAC AAATTTAAAC |
| 151 | ATTTATTTCC CTGCAAACCT CTTCTTTATT TTCAGAGTGT CTTTTCTTTT |
| 201 | TTGCTACTAG AGTAAAAGAA GTGGAAGAGA TGTGTAAGCG CAGCCTTGAG |
| 251 | TCTGTGTATT CGCTCTATCC CACACTTAGC AGGTTGCAGG CCATTGGAGA |
| 301 | GCTGGAAAGC ATTGGGGAGC TTTTCTCAAGGTATGTAATT CGTATGACTT |
| 351 | TGTTATCCTA AAGTGCAGCT TTTCTGTTAC CAATAGTGAC TTTAAAAAAT |
| 401 | AAAAACTATA GGCCGGGCAC GGTGGCTCAT GCCTGTAATC CTAGCACTTT |
| 451 | AGAAGGCTGA AGTGGGTGGA TCACTTGAGG TCAGGAGTTC AAGACCAGCC |
| 501 | TGGCCAACAT GGT |

************************************************************

| | |
|---|---|
| Exons 48-49 | (SEQ ID No:49) |

| | |
|---|---|
| 1 | TGTATTATTA TAATATTATA TCGTAAGTTC CAGGACTTAC ATAGTTTTTT |
| 51 | TTTTTTTTTT TTCATTTCTC TTGCTTACAT GAACTCTATG TCGTGGCATT |
| 101 | CAGATCAGTC ACACATAGAC AACTCTCTGA AGTATATATT AAGTGGCAGA |
| 151 | AACACTCCCA GCTTCTCAAG GACAGTGATT TTAGTTTTCA GGAGCCTATC |
| 201 | ATGGCTCTAC GCACAGTCAT TTTGGAGATC CTGATGGAAA AGGAAATGGA |
| 251 | CAACTCACAA AGAGAATGTA TTAAGGACAT TCTCACCAAA CACCTTGTAG |
| 301 | AACTCTCTAT ACTGGCCAGA ACTTTCAAGA ACACTCAGGT AAATACAATT |
| 351 | TAAAACTATG TCATCTTACC TCTTGACTTT CCTTTTATTA TTTAAAAAAC |
| 401 | TGAAAGCCTG AGGGAAAAAG AAATGTCATT AAGAGATAGA GATCTCTATT |
| 451 | AATATATAGT AAAAATAATT GTTTAAGAGT TCCCATTTTG GAATTAGATC |
| 501 | TGACTTTTAA GCCTTGGGCA AGGGTACTTA ATCTTTTCTC AACCTCAATT |
| 551 | TCCTGGTTAT AAAATGAGAA GATACCTAAC TTACTATATT GATAACAATT |
| 601 | CAGTGATTTT ATATACTGTG TGTATGTACA CACAGATACA CATACATACA |
| 651 | TATAGAGAGA GACAGACAGA CAGACAGATA GGCAGACGTG GGGTGGGGAG |
| 701 | ATTGTCAATG CAGACAGAGA GGGTCCTTAA AGATAGTCCC TGACAAGTAG |
| 751 | TTAAAGTCCT CAAATGAATG GTAGTTGCTG CTTTCATTAT TATTATTATT |
| 801 | CAAGGTAGTA GTATCAAGTA GTAAAAGTAT TTATTCCCAT ATGTCATTTT |
| 851 | CATTTCAGCT CCCTGAAAGG GCAATATTTC AAATTAAACA GTACAATTCA |
| 901 | GTTAGCTGTG GAGTCTCTGA GTGGCAGCTG GAAGAAGCAC AAGTATTCTG |
| 951 | GGCAAAAAAG GAGCAGAGTC TTGCCCTGAG TATTCTCAAG CAAATGATCA |
| 1001 | AGAAGTTGGA TGCCAGCTGT GCAGCGGTTT GTTTTTTTTA TTGGCTGGAT |
| 1051 | TAGTGTTTTA CTGTTATTTA AAAAAACACA AATGTACTTT AAAATATTTT |
| 1101 | TAATAACAAT TTTATTAGAG CCTTGAAATT AGTAATTTAT TAACAAGATA |
| 1151 | TTGTAAAACT AGTCTTGAAA ATTAATTTGT AAATGAAGTT TAGAAACTTT |
| 1201 | TTCCTATATA TCACAATTCT ATCAGTCCAT CATGTGGTCG ATTCATTTAA |
| 1251 | TATA |

************************************************************

| | |
|---|---|
| Exon 50 | (SEQ ID No:50) |

| | |
|---|---|
| 1 | TAATAATAAT AAACAGAGGA TGATCATTTC CtACATGGGA TTATTAAAAT |
| 51 | AGTTGTATGG CAAAAGCAGA TGAGGAAAAA CTTTTTTTTT CCCACCCACC |
| 101 | AAGGAAAAAC ATTTTTAACC TGCTTTTTTC CCCCGTACAT GAAGGGCAGT |
| 151 | TGGGTACAGT CATGGTAATG CATTATATTT TAAGATTTTG CCTTTCTTAT |
| 201 | ACAGAACAAT CCCAGCCTAA AACTTACATA CACAGAATGT CTGAGGGTTT |
| 251 | GTGGCAACTG GTTAGCAGAA ACGTGCTTAG AAAATCCTGC GGTCATCATG |
| 301 | CAGACCTATC TAGAAAAGGT AAGATTTTTG GAGCAACCCT TAAGATAGTT |
| 351 | ACTTAGCATG AATATGCTTC ATCTTTTCAT CAAGATCAAT ATATTTCCAA |
| 401 | AGCAAATAAA AGTATGGTTT TATTTTTCTA TATATTATTA CTGTTGTAGC |
| 451 | TCTGTATAGT CTCTAGGGTG GAGTGAAACA TTGTTACAAA ACAAAGCAGC |
| 501 | CAATTTGAAA AGTAAGCCCA AGTATAGTAT CTCTTCT |

************************************************************

| | |
|---|---|
| Exon 51 | (SEQ ID No:51) |

| | |
|---|---|
| 1 | TTTAAGAAAA TGTACGAATT TGTGTTGGGC CACATTCAAA GCCGTCCTGG |
| 51 | GCCACATGCG GCCCATGGGC CGTGGGTTGG ACAAGTTTGC AATAGTTCAT |
| 101 | ATAATTTAGC TAGCTTTTAT ATGTATATAA GTTAAATTTT AGTGTATTAC |
| 151 | CTTAATTTGA GTGATTCTTT AGATGTATTT AGTATTTGTA AATATAATTT |
| 201 | AAATTGGTTG TGTTTTCTTG AAGGCAGTAG AAGTTGCTGG AAATTATGAT |
| 251 | GGAGAAAGTA GTGATGAGCT AAGAAATGGA AAAATGAAGG CATTTCTCTC |
| 301 | ATTAGCCCGG TTTTCAGATA CTCAATACCA AAGAATTGAA AACTACATGA |
| 351 | AATCATCGGA ATTTGAAAAC AAGCAAGCTC TCCTGAAAAG AGCCAAAGAG |
| 401 | GAAGTAGGTC TCCTTAGGGA ACATAAAATT CAGACAAACA GGTAACTAGG |
| 451 | TTTCTACAAG TGACAATTTT ATGTTCACCA GTTAACTGAG TGAGTGTTTT |
| 501 | TGCATAGAAA GAGTGACTTG GTCTTTTTAT CTGATATAGT TTTGAGCTCT |
| 551 | AAAGGTCGGC TTAACTATAT ATAGATTATC TTGGTCTTTT GGGTTCTTTT |
| 601 | CGGTTTTTGT TTTTTGTGTT TTTTTTTGAG ACAAGGTCTC ACTCTCTCAC |
| 651 | CCAGGCTGGA GTACAgtGGc GTGATCACT |

************************************************************

| | |
|---|---|
| Exon 52 | (SEQ ID No:52) |

| | |
|---|---|
| 1 | TCCCCTTTGT CCTTTGATGC TTAGGAAGGT GTGTGAATTG CACAGTTAAG |
| 51 | ACAAAAGTAA GTTTATTCCC TTTATAATCC TTAGAAGTTT GCTTTTTTCC |
| 101 | CTGGGATAAA AACCCAACTT TTTTCATTAA ATGTTGTATA TCATGTGTGA |
| 151 | TTTTGTAGTT CTGTTAAAGT TCATGGCTTT TGTGTTTTAC CTTAATTATT |
| 201 | CTATGCAAGA TACACAGTAA AGGTTCAGCG AGAGCTGGAG TTGGATGAAT |
| 251 | TAGCCCTGCG TGCACTGAAA GAGGATCGTA AACGCTTCTT ATGTAAAGCA |
| 301 | GTTGAAAATT ATATCAACTG CTTATTAAGT GGAGAAGAAC ATGATATGTG |
| 351 | GGTATTCCGG CTTTGTTCCC TCTGGCTTGA AAATTCTGGA GTTTCTGAAG |
| 401 | TCAATGGCAT GATGAAGGCA AGTGTTACTC AGCCCAATAT TCTACCCTGT |
| 451 | GCTTGAAAAA CTTAGACATA AGCCCCTTGA TGTCAGGAAT CGTGTATACC |
| 501 | TCTTTGTATT CCTAGCACTT GGTCCAGTGC TCTACACATA AGTAGCATTT |
| 551 | TGTAGTTTTC TAAACTTTGA TCCATATTTA GGATTATTTA CAAGTTCTAG |
| 601 | TCTTGTTTCT |

************************************************************

| | |
|---|---|
| Exon 53-54 | (SEQ ID No:53) |

| | |
|---|---|
| 1 | AATCCAGTTT AATTTAGGAC CCAATATTTT GATTTACCAA TGCATTAATC |
| 51 | TAGAGTACCC ATTAGAAAGA CCTTCAGATA AGAAAAGAAA TGAAGGAAAA |
| 101 | CAATATAGTT AGTGAAGTTT TGTTAACCAC TTGTGCTAAT AGAGGAGGAC |
| 151 | TGTCTTAAAA TAACTTACTT GCTTAGATGT GAGAATATTT GAAATACCTT |
| 201 | GTTTCTTAAT TTTGTGTCTT TTTTTTAATG GTAGAGAGAC GGAATGAAGA |
| 251 | TTCCAACATA TAAATTTTTG CCTCTTATGT ACCAATTGGC TGCTAGAATG |
| 301 | GGGACCAAGA TGATGGGAGG CCTAGGATTT CATGAAGTCC TCAATAATGT |
| 351 | AAGTAAACCT GAAAATCAAA CCACAATAAT TATTTTTATT CTATTATTAC |
| 401 | TATATATTAT ATAAAGTATA TATACCATTC CCTCTAAGAA ATGGAAATAC |
| 451 | AAAATTTTGT ATTTTTTGTC TTCTCACATC ACATAAGTTA CTCATTTTCT |
| 501 | CTCTCTAATT CCTCATAGGC CTCTGCCTTT TTCTCACACA TGCAGGCATA |
| 551 | CACGCTCTAC CCACTGCAGT ATCTAGACAG TAATACACAT TTTAATGTTA |
| 601 | AGCAAAATGA AAAATATGGA TTATATTTTT TTGTTTATTT GCATAAATCT |
| 651 | AATAGTTCTT TTCTTACAGC TAATCTCTAG AATTTCAATG GATCACCCCC |
| 701 | ATCACACTTT GTTTATTATA CTGGCCTTAG CAAATGCAAA CAGAGATGAA |
| 751 | TTTCTGACTA AACCAGAGGT AGCCAGAAGA AGCAGAATAA CTAAAAATGT |
| 801 | GCCTAAACAA AGCTCTCAGC TTGATGAGGT ATTGGGATTA ACCATACGTA |
| 851 | CCTTTTAGAA GTGTGATATT CAGTCTTTCC TAGAATATTT CTTTTTAAAA |
| 901 | TCTTGTGTTA TTAAGATGCC ATCTAAAATC GGTTCAAGGC TGGCACGGTG |
| 951 | GCTCACGCCT GTAATCCCAG CACTTTGGGA GGCTGAGG |

************************************************************

| | |
|---|---|
| Exon 55 | (SEQ ID No:54) |

| | |
|---|---|
| 1 | GAAAGGCACC TAAGTCATTG ACGAGAGTAT GTATCTTTGA TGTATTTCAT |
| 52 | TTATGACTGT TTTGTTTGTA TCTGAGGAAT TATAATCATT CCATAGTCTA |
| 101 | GATTTGTGCA TAAATTCTGT TTTTCTCTTT GTTTTTCTAA CTCTGAGAAG |
| 151 | TTTAAATGTT GGGTAGTTCC TTATGTAATG TTTTTTGTTT TTTATTAATA |
| 201 | GGATCGAACA GAGGCTGCAA ATAGAATAAT ATGTACTATC AGAAGTAGGA |
| 251 | GACCTCAGAT GGTCAGAAGT GTTGAGGCAC TTTGTGATGC TTATATTATA |
| 301 | TTAGCAAACT TAGATGCCAC TCAGTGGAAG ACTCAGAGAAGTATGTTTTT |
| 351 | TTTAAAGAAG AAACGTTACT TTCTTGCTGT GTTACTCTCT GTAGAGATAT |
| 401 | ATTAGTTATA GAGCCTAATA AGTAAATCTG CTTAAAATCA CAAACGTAAT |
| 451 | CCAAAAGCTT AATTTATATC TGATGGCTTC AACATTCCCT GGTTACTTTT |
| 501 | TCACTTAATA TCTCTTAATA GAACTGGTAA TAGGTGA |

************************************************************

| | |
|---|---|
| Exon 56 | (SEQ ID No:55) |

| | |
|---|---|
| 1 | CTGTTGAGCT TTGACTCTGA GCTGCATAGT GGCCAAAGCC CAGAGTCTTC |
| 51 | ATTTCTCAAT CAGAGCCTGA ACCACAGATT AGCAACAAGT TGGGGCCAGT |
| 101 | GGTATCTGCT GACTATTCCT GCTTGACCTT CAATGCTGTT CCTCAGTTTG |
| 151 | TCACTAAAAT CTCTTCATTT TTAAATACAG AAGGCATAAA TATTCCAGCA |
| 201 | GACCAGCCAA TTACTAAACT TAAGAATTTA GAAGATGTTG TTGTCCCTAC |
| 251 | TATGGAAATT AAGGTAATTT GCAATTAACT CTTGATTTTT TTTAAACTAA |
| 301 | ATTTTTTTTA TTAGATTGAA CCATTTGAAA TAGTATTTTT ATGTAGGTCA |
| 351 | AAATTGGTTA AATATTGGCA AATTTCATAT GTTTCAACCT ATAATTTCTC |
| 401 | AGTATTATAT TTCCTTTGCC C |

************************************************************

| | |
|---|---|
| Exon 57 | (SEQ ID No:56) |

| | |
|---|---|
| 1 | ATATTAGTGA ATCTTTGATG AAACAGTAGT TAAAGTTACG AGCGTGAGCC |
| 51 | ACCACACCCG GCCTAAAGTT GTAGTTCTTA ACCACTATCA CATCGTCATT |
| 101 | TGTTTCTCTG TTTAATATTA AAATTGCCAT TTATAATGTA TTGTGCTTTA |
| 151 | AGTGCAAATA GTGTATCCGA CCTATTAGCA ATCATGTTTA TACTTTTATT |
| 201 | AGGTGGACCA CACAGGAGAA TATGGAAATC TGGTGACTAT ACAGTCATTT |
| 251 | AAAGCAGAAT TTCGCTTAGC AGGAGGTGTA AATTTACCAA AAATAATAGA |
| 301 | TTGTGTAGGT TCCGATGGCA AGGAGAGGAG ACAGCTTGTT AAGGTGAGCC |
| 351 | TTCCCTTCTC TGGCTTAGCC CTTAGAGTTT TAGTGATGAA AATTTTTAGT |
| 401 | TCATATTTCT TTCTGCTTTA TTGGGGATTT GGGTCTTTAT TTGGGAATA |

************************************************************

| | |
|---|---|
| Exon 58 | (SEQ ID No:57) |

| | |
|---|---|
| 1 | GGGAAACTTT CTAAATCAGT GTAAATGTTG TAGCTTATTC TAAATGAAAG |
| 51 | AATGGCAGTA GGTATTTAAT TATTTGGGAG ACTGTCAAGA GGTGCACAGA |
| 101 | TGCTCAGATT GGTTTGAGTG CCCTTTGCTA TTCTCAGATG ACTCTGTGTT |
| 151 | TTTATAATAA AATAAACTGT ACTTGTTTAT TCATGCTTAA TTATTCTGAA |
| 201 | GGGCCGTGAT GACCTGAGAC AAGATGCTGT CATGCAACAG GTCTTCCAGA |
| 251 | TGTGTAATAC ATTACTGCAG AGAAACACGG AAACTAGGAA GAGGAAATTA |
| 301 | ACTATCTGTA CTTATAAGGT AACTATTTGT ACTTCTGTTA GTTCACCAAA |
| 351 | AACATATAAA AGATGCCATT TGGTTGGGTG AAGTGGCTCA TGCCCATATT |
| 401 | CATAATGCTT TGGGAGGCCA AGGTGGGAGG ATTGCTTGAG GCCAGGAGTT |
| 451 | CGAGACCAGC CTCAGCAACA TAGTGAGACC CCATCTTGAC AAAAAGTTAA |
| 501 | AAAAAAAAAA AAAACCAGAG |

************************************************************

| | |
|---|---|
| Exon 59 | (SEQ ID No:58) |

| | |
|---|---|
| 1 | TTCTTTGAGC TTAAGTTTAT TTCCGATTGG TTTCCTCCAA GGAGCTTTGT |
| 51 | CTTCTATGGA CAGAGAAATA TTAATACAAC TTGAAAAAAA ATGCTTTGCA |
| 101 | CTGACTCTGA TAGCTGAATG ATCATCAAAT GCTCTTTAAT GGCCTTTTAA |
| 151 | AAGTAAAAGG TATTTAATCT GTAACTCCAG GTGGTTCCCC TCTCTCAGCG |
| 201 | AAGTGGTGTT CTTGAATGGT GCACAGGAAC TGTCCCCATT GGTGAATTTC |
| 251 | TTGTTAACAA TGAAGATGGT GCTCATAAAA GATACAGGCC AAATGATTTC |
| 301 | AGTGCCTTTC AGTGCCAAAA GAAAATGATGGTGAGTGACA CCCAAAATTA |
| 351 | AAGGTTATTG TAAGATTATT TAATGGCTTA TTAAAGCTGA CAGCTGTCAG |
| 401 | ATATTATAGA ATACAAAAAA ACTTTAATTT CATCAGGTAA TTGTCAAAGA |
| 451 | TACTAAGTAA AAGAAAAACT CATCAGAATG AAAGTGTGTG AGTGAAAAAG |
| 501 | GAAGGATTTT AAACTACAT |

************************************************************

| | |
|---|---|
| Exon 60 | (SEQ ID No:59) |

| | |
|---|---|
| 1 | ATCACTTGTA ATTAATTGCT TCCCTGTCCA GACTGTTAGC TTCTTGTAGG |
| 51 | TAATGTATCC TGTTCATCTT TATTGCCCCT ATATCTGTCA TATTTTTATA |
| 101 | TAAAAATGTG TATATTAGTT TAATTGAACA CAATATTGAA AAATAATTAT |
| 151 | ATATATTCTC TATTTAAAGG AGGTGCAAAA AAAGTCTTTT GAAGAGAAAT |
| 201 | ATGAAGTCTT CATGGATGTT TGCCAAAATT TTCAACCAGT TTTCCGTTAC |
| 251 | TTCTGCATGG AAAAATTCTT GGATCCAGCT ATTTGGTTTG AGAAGCGATT |
| 301 | GGCTTATACG CGCAGTGTAG CTACTTCTTC TATTGGTAAT CTTCTTGTAC |
| 351 | ATATAGTAGA TTGAGCACTT TGTTGTTTGG CAGGTTTTAT TTTTGTTTGA |
| 401 | TTCAGCACTT TTTCTACATT CTGAGTTGCA GGGGGATGAT AGTGATGATG |
| 451 | TGGTTAGTAA CCATCCCATC TTCATTATTA AATCATATGT TTCTTGTTCA |
| 501 | TCCTGATTCT TAGTGTCTAC CTTTTTATAA CTTATGCAGA AGAGAATTCT |

************************************************************

| | |
|---|---|
| Exon 61 | (SEQ ID No:60) |

| | |
|---|---|
| 1 | AATCATCTAG GATTTGTAAA ATGCAATATG CATTAAAATA GCTGGCAAGA |
| 51 | TTTGAGTTAA ACTCaACATG GCCGGTTATG CACATCATTT AAGTAGGCTA |
| 101 | AAAATCCTAA ACTACTTAAA GATTATACCA AGTCAGTGGT CTTAATTGAA |
| 151 | ATTATGGCTA TATATTAGAA AGAGATGGAA TCAGTGATTT CAGATTGTTT |
| 201 | GTTTCTTTTT TCTCCAGTTG GTTACATACT TGGACTTGGT GATAGACATG |
| 251 | TACAGAATAT CTTGATAAAT GAGCAGTCAG CAGAACTTGT ACATATAGAT |
| 301 | CTAGGTAAGT AATAAAATCT ATGTATCTAT TCTTTTTAGT AAATATTTGG |
| 351 | TCATCATGGA ATGTTGTTTG CCTACCAAGA TATTACAAAT ATAAGAGACA |
| 401 | GATAAATCGA AGCAGTAAAT ATTGGGTTTT TTTGTTTTCA GCATAAACAG |
| 451 | TTGTCCTAGA AGAAACAGTT AACT |

************************************************************

| | |
|---|---|
| Exon 62 | (SEQ ID No:61) |

| | |
|---|---|
| 1 | GATTTGAGGT GGATCTCACA GACAGTGACA AAGATGAGGA AGGCAGCCAG |
| 51 | AGCAGAAGTA AACTACTGTA CATACTAGTG TTCATAGAAC GTAGGTAACA |
| 101 | TGTGGTTTCT TGCCTTTGTA AAGTTCACAT TCTAACTGGA AAGAAAGTAA |
| 151 | ATTAGCTGTC AAACCTCCTA ACTTCACTGT ATTCTTTACT TTAGGTGTTG |
| 201 | CTTTTGAACA GGGCAAAATC CTTCCTACTC CTGAGACAGT TCCTTTTAGA |
| 251 | CTCACCAGAG ATATTGTGGA TGGCATGGGC ATTACGGGTG TTGAAGGTGT |
| 301 | CTTCAGAAGG TAAGTGATAT GAAGTAAAGG AGGGAAATAA TTTTTGATGT |
| 351 | CAAAATTACA TGGGCTGGGC ATGGTTCTTT GCACCTGTAA TCCCAGCTAC |
| 401 | TCAAGAGGCT GAAGTGGGAG GATTGTTTGA GCCCAGGAGT TTGAGTCCAG |
| 451 | CCTAGGCAAT ACAGCAAGAC CCTGTATCTA AAA |

************************************************************

| | |
|---|---|
| Exon 63 | (SEQ ID No:62) |

| | |
|---|---|
| 1 | TTCTTGTAAA TGCCAAGCTT GTGAAATAGT CAAATACATA TTTGTATTCA |
| 51 | TTTCAAACGT CTAATGAAAG CCCACTCTGC CAAGTATTAT GCTATTTTGA |
| 101 | GATACAGATA TGTAGATTAT TAAGCATAGG CTCAGCATAC TACACATGAG |
| 151 | AGTATACAGA TAAAGATATG TTGACAACAT TGGTGTGTAA CAAAATCCGT |
| 201 | ATTTATAATG TGTTTGACTC TAGATGCTGT GAGAAAACCA TGGAAGTGAT |
| 251 | GAGAAACTCT CAGGAAACTC TGTTAACCAT TGTAGAGGTA AAGTATTTTA |
| 301 | TAAGGAAGAC TTTATTTT |

************************************************************

| | |
|---|---|
| Exons 64-65 | (SEQ ID No:63) |

| | |
|---|---|
| 1 | AAGAACAGAT GTTCTCTCTG TTTAGGTCCT TCTATATGAT CCACTCTTTG |
| 51 | ACTGGACCAT GAATCCTTTG AAAGCTTTGT ATTTACAGCA GAGGCCGGAA |
| 101 | GATGAAACTG AGCTTCACCC TACTCTGAAT GCAGATGACC AAGAATGCAA |
| 151 | ACGAAATCTC AGGTGAGCAG TATTTTAAGA AGGTCCTGTT GTCAGTATTT |
| 201 | CAGATTTTCT TATTCCCAAG GCCTTTAAAC TGGTCACCTG GACTGGAACC |
| 251 | TTTGTGTTTT TGTCCTTAGT GATATTGACC AGAGTTTCGA CAAAGTAGCT |
| 301 | GAACGTGTCT TAATGAGACT ACAAGAGAAA CTGAAAGGAG TGGAAGAAGG |
| 351 | CACTGTGCTC AGTGTTGGTG GACAGGTGAA TTTGCTCATA CAGCAGGCCA |
| 401 | TAGACCCCAA AAATCTCAGC CGACTTTTCC CAGGATGGAA AGCTTGGGTG |
| 451 | TGA |

(stop codon) Exon 65 continues beyond the TGA stop

| | |
|---|---|
| codon into the 3′UTR | (SEQ ID No:8) |

************************************************************

Each exon and its flanking intron sequences are listed separately with the exon underlined.
Exons are separated by a row of ****.
Example primers are in bold.

### REFERENCES

Aicardi et al., “Ataxia-ocularmotor apraxia: A syndrome mimicking ataxia-telangiectasia” Ann. Neurol. 24:497-502 (1988).

Aksentijevitch et al., “Familial Mediterranean fever in Moroccan Jews: Demonstration of a founder effect by extended haplotype analysis” Am. J. Hum. Genet., 53:644-651 (1993).

Ambrose et al., “A physical map across chromosome 11q22-23 containing the major locus for ataxia-telangiectasia. Genomics, 21:612-619 (1994a).

Ambrose et al., 1994b. Structure and expression of the Huntington's disease gene: evidence against simple inactivation due to an expanded CAG repeat. Som. Cell Mol. Genet. 20:27-38.

Anderson and Kunkel, “The molecular and biochemical basis of Duchenne muscular dystrophy” Trends Riochem. Sci. 17:289-292 (1992).

Attree et al., “The Lowe's oculocerebrorenal syndrome gene encodes protein highly homologous to inositol polyphosphate-5-phosphatase” Nature, 358:239-242 (1992).

Ballabio et al., “Molecular heterogeneity of steroid sulfatase deficiency: a multicenter study on 57 unrelated patients, at DNA and protein levels” Genomics 4:36-40 (1989).

Barker, “A more robust, rapid alkaline denaturation sequencing method”, BioTechniques, 14(2):168-169 (1993).

Barnes, 1994. PCR amplification of up to 35-kb DNA with high fidelity and high yield from lambda bacteriophage templates. Proc. Natl. Acad. Sci. 91:2216-2220.

Berger et al., “Isolation of a candidate gene for Norrie disease by positional cloning” Nature Genet. 1:199-203, (1992)

Beaudet and Tsui, “A suggested nomenclature for designating mutations” Hum. Mutat. 2:245-248 (1993).

Broughton et al., “Mutations in the xeroderma pigmentosum group D DNA repair/transcription gene in patients with trichothiodystrophy” Nature Genet. 7:189-194 (1994).

Broughton et al., “Molecular and cellular analysis of the DNA repair defect in a patient in xeroderma pigmentosum group D who has the clinical features of xeroderma pigmentosum and Cockayne's syndrome” Am. J. Hum. Genet. 56:167-174 (1995).

Brown et al., “Control of p70 S6 kinase by kinase activity of FRAP in vivo” Nature 377:441-446 (1995).

Buckler et al., “Exon amplification: a strategy to isolate mammalian genes based on RNA splicing” Proc. Natl. Acad. Sci. USA, 88:4005-4009 (1991).

Burke and Olson, “Preparation of Clone Libraries in Yeast Artificial-Chromosome Vectors” in Methods in Enzymolocy, Vol. 194, “Guide to Yeast Genetics and Molecular Biology”, eds. C. Guthrie and G. Fink, Academic Press, Inc., Chap. 17, pp. 251-270 (1991).

Byrne et al., “Ataxia-without-telangiectasia” J Neurol. Sci. 66:307-317 (1984).

Capecchi, “Altering the genome by homologous recombination” Science 244:1288-1292 (1989).

Chakravarti et al., “Nonuniform recombination within the human beta-globin gene cluster” Am. J. Hum. Genet., 36:1239-1258 (1984).

Chelly et al., “Isolation of a candidate gene for Menkes disease that encodes a potential heavy metal binding protein” Nature Genet. 3:14-19 (1993).

Cheng et al., 1994. Effective amplification of long targets from cloned inserts and human genomic DNA. Proc. Natl. Acad. Sci. 91:5695-5699.

Chessa et al., “Heterogeneity in ataxia telangiectasia: classical phenotype associated with intermediate cellular radiosensitivity” Am. J. Med. Genet. 42:741-746 (1992).

Chillon et al., “Mutations in the cystic fibrosis gene in patients with congenital absence of the vas deferens” New Enql. J. Med. 332:1475-1480 (1995).

Church et al., “Isolation of genes from complex sources of mammalian genomic DNA using exon amplification” Nature Genet. 6:98-104 (1993).

Church et al., 1994. Isolation of genes from complex sources of mammalian genomic DNA using exon amplification. Nature Genet. 6:98-94.

Collins, F. S. “Positional cloning: let's not call it reverse anymore” Nature Genet., 1:3-6 (1992).

Cooper and Krawczak, Human gene mutation. BIOS Scientific Publishers, London (1993).

Davies et al., “Targeted alterations in yeast artificial chromosomes for inter-species gene transfer”, Nucleic Acids Research, 20(11):2693-2698 (1992).

Derry et al., “WSP gene mutations in Wiskott-Aldrich syndrome and X-linked thrombocytopenia” Hum. Mol. Genet. 4:1127-1135 (1995).

Dickinson et al., “High frequency gene targeting using insertional vectors”, Human Molecular Genetics, 2(8):1299-1302 (1993).

Dietz and Kendzior, “Maintenance of an open reading frame as an additional level of scrutiny during splice site selection” Nature Genet. 8:183-188 (1994).

Duyk et al., “Exon trapping: A genetic screen to identify candidate transcribed sequences in cloned mammalian genomic DNA” Proc. Natl. Acad. Sci. USA, 87:8995-8999 (1990).

Fiorilli et al., “Variant of ataxia-telangiectasia with low-level radiosensitivity” Hum. Genet. 70:274-277 (1985).

Fodor et al, “Multiplexed biochemical assays with biological chips”, Nature 364:555-556 (1993)

Foord and Rose, 1994. Long-distance PCR. PCR Methods Appl. 3:S149-S161.

Foroud et al. “Localization of the AT locus to an 8 cM interval defined by STMY and S132” Am. J. Hum. Genet., 49:1263-1279 (1991).

Friedman and Weitberg, “Ataxia without telangiectasia” Movement Disorders 8:223-226 (1993).

Frohman, M. A. “On beyond classic RACE (rapid amplification of cDNA ends)” PCR Methods and Applications, 4:S40-S58 (1994).

Frohman et al., “Rapid production of full-length cDNAs from rare transcripts: Amplification using a single gene-specific oligonucleotide primer” Proc. Natl. Acad. Sci. USA, 85:8998-9002 (1988).

Gatti et al., “Genetic haplotyping of ataxia-telangiectasia families localizes the major gene to an 850 kb region on chromosome 11q23.1” Int. J. Radiat. Biol. (1994).

Gatti et al. “Localization of an ataxia-telangiectasia gene to chromosome 11q22-23” Nature, 336: 577-580 (1988).

Gibson et al., “A nonsense mutation and exon skipping in the Fanconi anaemia group C gene” Hum. Mol. Genet. 2:797-799 (1993).

Gilad et al., Hum. Mol. Genet. 5:433-439 (1996).

Gilboa et al. “Transfer and expression of cloned genes using retroviral vectors” BioTechniques 4(6):504-512 (1986).

Gottlieb and Jackson, “Protein kinases and DNA damage” Trends Biochem. Sci. 19:500-503 (1994).

Greenwell et al., “TEL1, a gene involved in controlling telomere length in Saccharomyces cerevisiae, is homologous to the human ataxia telangiectasia (ATM) gene” Cell 82:823-829 (1995).

Hastbacka et al., “Linkage disequilibrium mapping in isolated founder populations: diastrophic dysplasia in Finland” Nature Genet., 2:204-211 (1992).

Harding, “Clinical features and classification of inherited ataxias” Adv. Neurol. 61:1-14 (1993).

Harnden, “The nature of ataxia-telangiectasia: problems and perspectives” Int. J. Radiat. Biol. 66:S13-S19 (1994).

Hogervorst et al., “Rapid detection of BRCA1 mutations by the protein truncation test” Nature Genetics 10:208-212 (1995).

Huxley et al., “The human HPRT gene on a yeast artificial chromosome is functional when transferred to mouse cells by cell fusion”, Genomics, 9:742-750 (1991).

Jackson, 1991. A reappraisal of non-consensus mRNA splice sites. Nucleic Acids Res. 19:3795-3798.

Jakobovits et al., “Germ-line transmission and expression of a human-derived yeast artificial chromosome”, Nature 362:255-261 (1993).

James et al., “A radiation hybrid map of 506 STS markers spanning human chromosome 11”, Nature Genet. 8:70 (1994).

Jarvi et al., Cystic fibrosis transmembrane conductance regulator and obstructive azoospermia” The Lancet 345:1578 (1995).

Jaspers et al., “Genetic complementation analysis of Ataxia-Telangiectasia and Nijmegen breakage syndrome: A survey of 50 patients”, Cytogenet. Cell Genet., 49:259 (1988).

Kawasaki E S. Amplification of RNA. In: PCR protocols: A Guide to Methods and Applications, Innis M A, Gelfand D H, Sninsky J J, White T J, eds. Academic Press, 1990, pp21-27.

Kerem et al., “Identification of the cystic fibrosis gene: genetic analysis” Science, 245:1073-1080 (1989).

Kolluri et al., “Identification of WASP mutations in patients with Wiskott-Aldrich syndrome and isolated thrombocytopenia reveals allelic heterogeneity at the WAS locus” Hum. Mol. Genet. 4:1119-1126 (1995).

Kozak et al., “An analysis of 5′-noncoding sequences from 699 vertebrate messenger RNAs” Nucleic Acids Res., 15:8125-8148 (1987).

Lamb et al., “Introduction and expression of the 400 kilobase precursor amyloid protein gene in transgenic mice”, Nature Genetics, 5:22-29 (1993).

Lange et al., “Localization of an ataxia-telangiectasia gene to a 850 kb interval on chromosome 11q23.1 by linkage analysis of 176 families in an international consortium” Am. J. Hum. Genet. 57:112-119 (1995).

Lehesjoki et al., “Localization of the EPM1 gene for progressive myoclonus epilepsy on chromosome 21: linkage disequilibrium allows high resolution mapping” Hum. Mol. Genet., 2:1229-1234 (1993).

Lichter et al., “High-resolution mapping of human chromosome 11 by in situ hybridization with cosmid clones” Science 247:64-69 (1990).

Litt and Luty, “A hypervariable microsatellite revealed by in vitro amplification of a dinucleotide repeat within the cardiac muscle actin gene” Am. J. Hum. Genet., 44:397-401 (1989).

Liu and Sommer, “Restriction endonuclease fingerprinting (REF): a sensitive method for screening mutations in long, contiguous segments of DNA” BioTechniques 18:470-477 (1995).

Llerena et al., “Spontaneous and induced chromosome breakage in chorionic villus samples: a cytogenetic approach to first trimester prenatal diagnosis of ataxia-telangiectasia syndrome” J. Med. Genet., 26:174-178 (1989).

Lovett et al., “Direct selection: A method for the isolation of cDNA encoded by large genomic regions”, Proc. Natl. Acad. Sci. USA 88, 9628 (1991)

Maserati et al., “Ataxia-without-telangiectasia in two sisters with rearrangements of chromosomes 7 and 14” Clin. Genet. 34:283-287 (1988).

McConville et al., “Genetic and physical mapping of the ataxia-telangiectasia locus on chromosome 11q22-23” Int. J. Radiat. Biol. (1994).

McConville et al., “Paired STSs amplified from radiation hybrids, and from associated YACs, identify highly Polymorphic loci flanking the ataxia-telangiectasia locus on chromosome 11q22-23” Hum. Mol. Genet., 2:969-974 (1993).

McConville et al., “Fine mapping of the chromosome 11q22-23 region using PFGE, linkage and haplotype analysis; localization of the gene for ataxia telangiectasia to a 5 cM region flanked by NCAM/DRD2 and STMY/CJ52.75, phi2.22” Nucleic Acids Res., 18:4335-4343 (1990).

Miki et al. “A strong candidate for the breast and ovarian cancer susceptibility gene BRCA1” Science, 266:66-71 (1994).

Mitchison et al., “Fine genetic mapping of the Batten Disease locus (CLN3) by haplotype analysis and demonstration of allelic association with chromosome 16p microsatellite loci” Genomics, 16:455-460 (1993).

Morgan et al., “The selective isolation of novel cDNAs encoded by the regions surrounding the human interleukin 4 and 5 genes” Nucleic Acids Res., 20:5173-5179 (1992).

Nehls et al., 1994a. Exon amplification from complete libraries of genomic DNA using a novel phage vector with automatic plasmid excision facility: application to the mouse neurofibromatosis-1 locus. Oncogene 9:2169-2175.

Nehls et al., 1994b. The sequence complexity of exons trapped from the mouse genome. Current Biology 4:983-989.

Orita et al. Detection of polymorphisms of human DNA by gel electrophoresis as single-strand conformation polymorphisms. Proc Natl Acad Sci USA 1989; 86:2766-2770

Oskato et al., “Ataxia-telangiectasia: allelic association with 11q22-23 markers in Moroccan-Jewish patients. 43rd Annual Meeting of the American Society of Human Genetics, New Orleans, La. (1993).

Ozelius et al., “Strong alleleic association between the torsion dystonia gene (DYTI) and loci on chromosome 9q34 in Ashkenazi Jews” Am. J. Hum. Genet. 50:619-628 (1992).

Parimoo et al., “cDNA selection: Efficient PCR approach for the selection of cDNAs encoded in large chromosomal DNA fragments” Proc. Natl. Acad. Sci. USA, 88:9623-9627 (1991).

Pease et al., “Light-generated oligonucleotide arrays for rapid DNA sequence analysis”, Proc. Natl. Acad. Sci. USA 91(11):5022-5026 (1994)

Richard et al., “A radiation hybrid map of human chromosme 11q22-23 containing the Ataxia-Telangiectasia disease locus”, Genomics 17, 1 (1993).

Roberts et al., 1993. Exon structure of the human dystrophin gene. Genomics 16:536-538.

Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).

Rotman et al., “Three dinucleotide repeat polymorphisms at the ataxia-telangiectasia locus” Human Molecular Genetics (1994b).

Rotman et al., “A YAC contig spanning the ataxia-telangiectasia locus (groups A and C) on chromosome 11q22-23. Genomics (1994c).

Rotman et al., “Physical and genetic mapping of the ATA/ATC locus in chromosome 11q22-23” Int. J. Radiat. Biol. (1994d).

Rotman et al., “Rapid identification of polymorphic CA-repeats in YAC clones” Molecular Biotechnology (1995).

Savitsky et al., “A single gene with homologies to phosphatidylinositol 3-kinases and rad3+ is Mutated in all complementation groups of ataxia-telangiectasia” Science, 268:1749-1753 (Jun. 23, 1995a)

Savitsky et al., “The complete sequence of the coding region of the ATM gene reveals similarity to cell cycle regulators in different species” Hum. Mol. Genet. 4:2025-2032 (1995b).

Schedl et al., “A yeast artificial chromosome covering the tyrosinase gene confers copy number-dependent expression in transgenic mice”, Nature, 362:258-261 (1993).

Sirugo et al., “Friedreich ataxia in Louisiana Acadians: Demonstration of a founder effect by analysis of microsatellite-generated extended haplotypes” Am. J.Hum. Genet., 50:559-566 (1992).

Shiloh, “Ataxia-telangiectasia: closer to unraveling the mystery” European Journal of Human Genetics (1995)

Shiloh et al., Am. J. Hum. Genet. 55 (suppl.), A49 (1994a)

Shiloh, et al., 1994b. Genetic, physical and functional analysis of the ataxia-telangiectasia locus on chromosome 11q22-23. 44th Annual Meeting of the American Society of Human Genetics, Montreal. Am. J. Hum. Genet. 55:A49.

Sommer, “Recent human germ-line mutation: Inferences from patients with hemophilia B” Trends Gene. 11:141-147-(1995).

Steingrimsdottir et al., “Mutations which alter splicing in the human hypoxanthine-guanine phosphoribosyl-transferase gene” Nucleic Acids Res. 6:1201-1208 (1992).

Strauss et al., “Germ line transmission of a yeast artificial chromosome spanning the murine α₁ (I) collagen locus”, Science, 259:1904-1907 (1993).

Tagle et al., “Magnetic capture of expressed sequences encoded within large genomic segments” Nature, 361:751-753 (1993).

Taylor et al., “Genetic and cellular features of ataxia telangiectasia” Int. J. Radiat. Biol. 65:65-70 (1994).

Taylor et al., Variant forms of ataxia telangiectasia. J. Med. Genet. 24, 669-677 (1987).

The European Polycystic Kidney Disease Consortium, “The polycystic kidney disease 1 gene encodes a 14 kb transcript and lies within a duplicated region on chromosome 16” Cell, 77:881-894 (1994).

The Huntington's Disease Collaborative Research Group, “A novel gene containing a trinucleotide repeat that is expanded and unstable on Huntington's disease chromosomes” Cell, 72:971-983 (1993).

Trofatter et al., “A novel moesin-, ezrin-, radixin-like gene is a candidate for the neurofibromatosis 2 tumor suppressor” Cell, 72:791-800 (1993).

Vanagaite et al., “Physical localization of microsatellite markers at the ataxia-telangiectasia locus at 11q22-23. Genomics, 22:231-233 (1994a).

Vanagaite et al., “High-density microsatellite map of ataxia-telangiectasia locus” Human Genetics 95:451-453 (1995).

Vetrie et al., “The gene involved in X-linked agammaglobulinemia is a member of the src family of protein-tyrosine kinases” Nature, 361:226-233 (1993).

Weber and May, “Abundant class of human DNA polymorphisms which can be typed using the polymerase chain reaction” Am. J. Hum. Genet., 44:388-396 (1989).

Weemaes et al., “Nijmegen breakage syndrome: A progress report” Int. J. Radiat. Biol. 66:S185-S188 (1994).

Ying and Decoteau, “Cytogenetic anomalies in a patient with ataxia, immune deficiency, and high alpha-fetoprotein in the absence of telangiectasia” Cancer Genet. Cytocenet. 4:311-317 (1983).

Zakian, “ATM-related genes: What do they tell us about functions of the human gene?” Cell 82:685-687 (1995).

Ziv et al., “Ataxia-telangiectasia: linkage analysis in highly inbred Arab and Druze families and differentiation from an ataxia-microcephaly-cataract syndrome” Hum. Genet., 88:619-626 (1992).

Ziv et al., “The ATC (ataxia-telangiectasia complementation group C) locus localizes to 11q22-q23. Genomics, 9:373-375 (1991) .

Ziv et al., “Ataxia telangiectasia: a variant with altered in vitro phenotype of fibroblast cells” Mutation Res. 210:211-219 (1989).

1. A purified, isolated and cloned nucleic acid sequence encoding a gene, designated ATM, mutations in which cause ataxia-telangiectasia and polymorphisms thereof and which has a genomic organization as set forte in Tables 1 and 5.

2. A nucleic acid sequence as set forth in claim 1 having a cDNA sequence set forth in SEQ ID Nos: 2, 8 and 9.

3. A purified, isolated and cloned nucleic acid sequence according to claim 1 wherein the nucleic acid is mRNA.

4. The nucleic acid sequence of claim 1 wherein a mutation event selected from the group consisting of point mutations, deletions and insertions has occurred such that the resulting sequence is altered imparting ataxia-telangiectasia.

5. The nucleic acid sequence of claim 4 wherein the mutation events are those set forth in Table 3.

6. The nucleic acid sequence of claim 1 wherein a mutation event selected from the group consisting of point mutations, deletions and insertions has occurred such that th resulting amino acid sequence is altered imparting ataxia-telangiectasia.

7. The nucleic acid sequence of claim 1 wherein a mutation event selected from the group consisting of point mutations, deletions, insertions and rearrangements has occurred within the flanking sequences of ATM such that regulation of ATM is altered imparting ataxia-telangiectasia.

8. The nucleic acid sequence of claim 1 wherein a mutation event selected from the group consisting of point mutations, deletions, insertions and rearrangements has occurred within the regulatory sequences of ATM such that regulation of ATM is altered imparting ataxia-telangiectasia.

9. A vector comprising an expression control sequence operatively linked to the nucleic acid sequence of claim 1.

10. A host cell, wherein the host cell is selected from the group of suitable eucaryotic and procaryotic cells, which is transformed with the vector of claim 9.

11. The host cell of claim 10 wherein it is E. coli.

12. A purified protein as encoded by altered nucleic acid sequences as set forth in claim 4 which allow production of a protein product said protein product imparting ataxia-telangiectasia.

13. An antibody which specifically binds to a polypeptide of claim 12.

14. A method for detecting carriers of a defective gene which causes ataxia-telangiectasia, said method comprising the steps of:
a) isolating genetic material from an isolated cell sample from a test subject;
b) performing restriction endonuclease fingerprinting (REF) or sequence analysis; and
c) detecting the presence of the gene defect which causes ataxia-telangiectasia.

15. A method as set forth in claim 14 wherein the cell is a lymphocyte.

16. A method as set forth in claim 14 wherein the sample is amniotic fluid.

17. A method as set forth in claim 14 wherein the sample is chorionic villi.

18. A method of detecting a protein set forth in claim 12 which causes ataxia-telangiectasia including the step of:
assaying an isolated specimen for protein with an assay selected from the group consisting of immunohistochemical and immunocytochemical staining, ELISA, RIA, immunoblots, immunoprecipitation, Western blotting, functional assays and protein truncation test.

19. A method of detecting a mRNA as set forth in claim 3 and polymorphisms and mutations thereof including the steps of:
isolating a specimen containing nucleic acid; and
assaying the specimen for complementary mRNA with an assay selected from the group consisting of in situ hybridization, Northern blotting and reverse transcriptase-polymerase chain reaction.

20. A method of detecting a nucleic acid sequence of claim 1 and polymorphisms and mutations thereof including the steps of:
isolating a specimen containing nucleic acid; and
assaying the specimen with an assay selected from the group consisting of in situ hybridization, Southern blotting, single strand conformational polymorphism, restriction endonuclease fingerprinting (REF), PCR amplification and DNA-chip analysis using nucleic acid sequence of claim 1 and polymorphisms and mutations thereof specific primers.

21. A method of screening for homozygous or heterozygous carriers of ATM mutant genes as set forth in claim 4 for genetic counseling including the step of:
assaying an isolated specimen from a potential carrier for mutations of the ATM gene.

22. A kit for detecting a nucleic acid sequence, ATM gene, of claim 1 and polymorphisms and mutations thereof in a population which cause ataxia-telangiectasia said kit including:
a molecular probe including PCI primers for a non-mutant ATM gene
at least one molecular probe including PCR primers for a genetic sequence ot a detective AIM gene which causes ataxia-telangiectasia in a population, and
detection means for indicating the presence of the defective allele

23. A kit for detecting a defective gene product associated with mutations of ataxia-telangiectasia in a population as set forth in claim 12, said kit comprising:
an antibody which recognizes mutant epitopes on the ATM gene product encoded by a mutant gene sequence which are associated with ataxia-telangiectasia in a population and
detection means for detecting the binding of the antibody to the gene product in a sample thereby indicating the presence of the defective gene product.

24. A method of screening subjects presenting with “partial A-T phenotypes” for ATM mutant genes for genetic counseling including the step of:
assaying an isolated specimen from a subject in a population presenting with a “partial A-T phenotype” specimen for mutations of the ATM gene wherein a nucleic acid sequence of claim 1 and polymorphisms and mutations thereof are identified by restriction endonuclease fingerprinting (REF).

25. A method of detecting a mRNA sequence and polymorphisms and mutations thereof including the steps of:
extracting from an isolated specimen mRNA;
performing reverse transcription on the mRNA
amplifying a selected open reading frame from the mRNA using RT-PCR obtained products;
amplifying the resulting products using nested PCR primers and obtaining second products;
subjecting the second products to restriction endonuclease fingerprinting (REF).

26. A method of detecting a nucleic acid sequence of claim 3 and polymorphisms and mutations thereof including the steps of:
extracting from an isolated specimen mRNA;
performing reverse transcription on the mRNA amplifying the ATM open reading frame (SEQ ID No: 2) from the mRNA using RT-PCR obtaining products;
amplifying the resulting products using nested PCR primers of SEQ ID Nos: 64-65 obtaining second products;
subjecting the second products to restriction endonuclease fingerprinting (REF) with primers of SEQ ID Nos: 66-81.

27. A method of detecting a genomic DNA nucleic acid sequence of claim 1 and polymorphisms and mutations thereof including the steps of:
isolating genomic DNA from a sample;
amplifying the genomic DNA using PCR with primers designed based on the exon-intron genomic structure of the ATM gene as set forth in Tables 1 and 5.


SEQUENCE LISTING (91 sequences)

SEQ ID NO: 1 - DNA (cDNA), 5912 bp; single-stranded, linear
  immediate source clone: 7-9
catactttttcctcttagtctacaggttggctgcatagaagaaaaaggtagagttatttataatcttgtaaatcttggactttgagtcatctattttcttttacagtcatcgaatacttttggaaataaggtaatatatgccttttgagctgtcttgacgttcacagatataaaatattaaatatattttaattttgtgcccttgcagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcgacaaagtagctgaacgtgtcttaatgagactacaagagaaactgaaaggagtggaagaaggcactgtgctcagtgttggtggacaggtgaatttgctcatacagcaggccatagaccccaaaaatctcagccgacttttcccaggatggaaagcttgggtgtgatcttcagtatatgaattaccctttcattcagcctttagaaattatattttagcctttatttttaacctgccaacatactttaagtagggattaatatttaagtgaactattgtgggtttttttgaatgttggttttaatacttgatttaatcaccactcaaaaatgttttgatggtcttaaggaacatctctgctttcactctttagaaataatggtcattcgggctgggcgcagcggctcacgcctgtaatcccagcactttgggaggccgaggtgagcggatcacaaggtcaggagttcgagaccagcctggccaagagaccagcctggccagtatggtgaaaccctgtctctactaaaaatacaaaaattagccgagcatggtggcgggcacctgtagtcccagctactcgagaggctgaggcaggagaatctcttgaacctgggaggtgaaggttgctgtgggccaaaatcatgccattgcactccagcctgggtgacaagagcgaaactccatctcaaaaa

SEQ ID NO: 2 - DNA (cDNA), 9171 bp, Homo sapiens; single-stranded, linear
  chromosome/segment: 11q22-23
atgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtga

SEQ ID NO: 3 - protein, 3056 aa, Homo sapiens [sequence omitted]

SEQ ID NO: 4 - protein, 15 aa
HEPANSSASQSTDLC

SEQ ID NO: 5 - protein, 15 aa
CKRNLSDIDQSFDKV

SEQ ID NO: 6 - protein, 18 aa
PEDETELHPTLNADDQEC

SEQ ID NO: 7 - protein, 26 aa
CKSLASFIKKPFDRGEVESMEDDTNG

SEQ ID NO: 8 - DNA (cDNA), 3607 bp, Homo sapiens; single-stranded, linear
  3′UTR 1..3607
tcttcagtatatgaattaccctttcattcagcctttagaaattatattttagcctttatttttaacctgccaacatactttaagtagggattaatatttaagtgaactattgtgggtttttttgaatgttggttttaatacttgatttaatcaccactcaaaaatgttttgatggtcttaaggaacatctctgctttcactctttagaaataatggtcattcgggctgggcgcagcggctcacgcctgtaatcccagcactttgggaggccgaggtgagcggatcacaaggtcaggagttcgagaccagcctggccaagagaccagcctggccagtatggtgaaaccctgtctctactaaaaatacaaaaattagccgagcatggtggcgggcacctgtaatcccagctactcgagaggctgaggcaggagaatctcttgaacctgggaggtgaaggttgctgtgggccaaaatcatgccattgcactccagcctgggtgacaagagcgaaactccatctcaaaaaaaaaaaaaaaaaacagaaacttatttggatttttcctagtaagatcactcagtgttactaaataatgaagttgttatggagaacaaatttcaaagacacagttagtgtagttactatttttttaagtgtgtattaaaacttctcattctattctctttatcttttaagcccttctgtactgtccatgtatgttatctttctgtgataacttcatagattgccttctagttcatgaattctcttgtcagatgtatataatctcttttaccctatccattgggcttcttctttcagaaattgtttttcatttctaattatgcatcatttttcagatctctgtttcttgatgtcatttttaatgtttttttaatgttttttatgtcactaattattttaaatgtctgtacctgatagacactgtaatagttctattaaatttagttcctgctgtttatatctgttgatttttgtatttgataggctgttcatccagttttgtctttttgaaaagtgagtttattttcagcaaggctttatctatgggaatcttgagtgtctgtttatgtcatattcccagggctgttgctgcacacaagcccattcttattttaatttcttggctttagggtttccatacctgaagtgtagcataaatactgataggagatttcccaggccaaggcaaacacacttcctcctcatctccttgtgctagtgggcagaatatttgattgatgcctttttcactgagagtataagcttccatgtgtcccacctttatggcaggggtggaaggaggtacatttaattcccactgcctgcctttggcaagccctgggttctttgctccccatatagatgtctaagctaaaagccgtgggttaatgagactggcaaattgttccaggacagctacagcatcagctcacatattcacctctctggtttttcattcccctcatttttttctgagacagagtcttgctctgtcacccaggctggagtgcagtggcatgatctcagctcactgaaacctctgcctcctgggttcaagcaattctcctgcctcagcctcccgagtagctgggactacaggcgtgtgccaacacgcccggctaattttttgtatttttattagagacggagtttcaccgtgttagccaggatggtctcgatcgcttgacctcgtgatccaccctcctcggcctcccaaagtgctgggattacaggtgtgagccaccgcgcccggcctcattcccctcatttttgaccgtaaggatttcccctttcttgtaagttctgctatgtatttaaaagaatgttttctacattttatccagcatttctctgtgttctgttggaagggaagggcttaggtatctagtttgatacataggtagaagtggaacatttctctgtcccccagctgtcatcatataagataaacatcagataaaaagccacctgaaagtaaaactactgactcgtgtattagtgagtataatctcttctccatccttaggaaaatgttcatcccagctgcggagattaacaaatgggtgattgagctttctcctcgtatttggaccttgaaggttatataaatttttttcttatgaagagttggcatttctttttattgccaatggcaggcactcattcatatttgatctcctcaccttcccctcccctaaaaccaatctccagaactttttggactataaatttcttggtttgacttctggagaactgttcagaatattactttgcatttcaaattacaaacttaccttggtgtatctttttcttacaagctgcctaaatgaatatttggtatatattggtagttttattactatagtaaatcaaggaaatgcagtaaacttaaaatgtctttaagaaagccctgaaatcttcatgggtgaaattagaaattatcaactagataatagtatagataaatgaatttgtagctaattcttgctagttgttgcatccagagagctttgaataacatcattaatctactctttagccttgcatggtatgctatgaggctcctgttctgttcaagtattctaatcaatggctttgaaaagtttatcaaatttacatacagatcacaagcctaggagaaataactaattcacagatgacagaattaagattataaaagatttttttttggtaattttagtagagacagggttgccattgtattccagccttggcgacagagcaagactctgcctcaaaaaaaaaaaaaaaaaggttttgccaagctggaactctttctgcaaatgactaagatagaaaactgccaaggacaaatgaggagtagttagattttgaaaatattaatcatagaatagttgttgtatgctaagtcactgacccatattatgtacagcatttctgatctttactttgcaagattagtgatactatgccaatacactgctggagaaatcagaatttggagaaataagttgtccaaggcaagaagatagtaaattataagtacaagtgtaatatggacagtatctaacttgaaaagatttcaggcgaaaagaatctggggtttgccagtcagttgctcaaaaggtcaatgaaaaccaaatagtgaagctatcagagaagctaataaattatagactgcttgaacagttgtgtccagattaagggagataatagctttcccaccctactttgtgcaggtcatacctccccaaagtgtttacctaatcagtaggttcacaaactcttggtcattatagtatatgcctaaaatgtatgcacttaggaatgctaaaaatttaaatatggtctaaagcaaataaaagcaaagaggaaaaactttggacatcgtaaagactagaatagtcttttaaaaagaaagccagtatattggtttgaaatatagagatgtgtcccaatttcaagtattttaattgcaccttaatgaaattatctattttctatagattttagtactattgaatgtattactttactgttacctgaatttattataaagtgtttttgaataaataattctaaaagcaaaaaaaaaaaaaaaaa

SEQ ID NO: 9 - DNA (cDNA), 884 bp, Homo sapiens; single-stranded, linear
  5′UTR 1..884
tcctccttttaaacgccctgaattgaaccctgcctcctgcgcatcctctttttgtgtcaccttagggttcagatttaactacgcgacttgactagtcatcttttgatctctctctcgtatttagtacttttagtcagcgagcatttattgatatttcaacttcagcctcgcggttaagagcttgggctctggaatcatacggctggaattggaattctgtcagtcgtgtggccgctctctactgtcttgtgaagataagtgagataatcttgacctgtggtgagcactcgtgagcgttagctgctgtatttaccaggtacagataagacaactacagtggatgataatgtatgtggtgataggggagtactctgatggtagaggagtgactttggttctctgcaaactcagcctgagactatcaattcagtttgtggtgagacctcgcagtgttaccttggcagatggtagaagccttccagatggaaggaaaaatgcgtgtaaaggcacaaagtgtagaaggaccctgaagctccagcgtgaggcctggcattgaatgaaatatattttgtgggttttcagctgctgaagtcataggaatggatgagaccaagaaaacaaagctgtttttgaggtatgagcggaagaagagatatcaggagactttcgaaacagtcataacggaagttaatatgatcattgctaacatttgctgtgtttcaggcactgtaagcatgtatatgggtccttaaagggactcatagagaggcatacatcacaatttggaattatgcattggtttatcaatttacttgtttattgtcaccctgctgcccagatatgacttcatgaggacagtgatgtgtgttctgaaattgtgaacc

SEQ ID NO: 10 - DNA (cDNA), 120 bp; single-stranded, linear
aggtagctgcgtggctaacggagaaaagaagccgtggccacgggaggaggcgagaggagtcgggatctgcgctgcagccaccgccgcggttgatactactttgaccttccgagtgcagtg

SEQ ID NO: 11 - DNA (genomic), 198 bp; single-stranded, linear
  exon 1..88
aggcatacatcacaatttggaattatgcattggtttatcaatttacttgtttattgtcaccctgctgcccagatatgacttcatgagggtaggatttgtatctgtttagttcattatttgtctagctataagtagtaaatattgtttgcaactatcagtgaatgagcatcttctgtttatgtagataatactgaactg

SEQ ID NO: 12 - DNA (genomic), 750 bp; single-stranded, linear
ttacagcattacttgtatagattttaaggagatctcattttaaatacggaaatgttaagaaaaattattgtgcctttgaccagaatgtgcctctaattgtacagataaatctaactataaatgctgcagtataaaataattacatacacattttttcacacctctttctctctatatatgcatatatacatacacatatatatacctatatgtattttttttacagacagtgatgtgtgttctgaaattgtgaaccatgagtctagtacttaatgatctgcttatctgctgccgtcaactagaacatgatagagctacagaacgaaaggtagtaaattacttaaattcaatttttccttgaaatgtgtgattagtaacccattattatttcctttttattttcagaaagaagttgagaaatttaagcgcctgattcgagatcctgaaacaattaaacatctagatcggcattcagattccaaacaaggaaaatatttgaattgggatgctgtttttaggtattctattcaaatttattttactgtctttatttttctctttcatatttatttctgttgtgatattacttttgtgtgtaagtcttaacatttatctttgattcctatatatcattatgccttgcatatgaatttggcatttaatatttatccaaaacataatttttaaaggttgttcatatagaaacttaaaaattataaattatttcttcaataaaatgttttagacata

SEQ ID NO: 13 - DNA (genomic), 388 bp; single-stranded, linear
gcagtaaagcaatagaaagtcatagaagattaagagctttgcagaccagatattaaattggtcttgtaggagttaggccttgaaagagagatttaattgttttatttgtttttttcagctgatgtagtaatctaagcaaggtggtttaaaagttgctctttgtgatggcatgaacagcttttgaaattattataatttaagtattcaacgagtttctgaaattgcattttgttttcttgaagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaa

SEQ ID NO: 14 - DNA (genomic), 349 bp; single-stranded, linear
gagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttaggtatgttttgaaggttgttgtttgtgaatttttcctcatgaaatgaaacttcaccaaagaaagcactctgtctgtatctgtctatatcccccaagtgacctgacaggtttaacagtactttagtaaaattatatggttatcgaactgacccttaatttttatttattatgtagcttttgaataa

SEQ ID NO: 15 - DNA (genomic), 388 bp; single-stranded, linear
aattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgaggtaatctaatctctttttctttgttttgtattgaaatacttttgatcttgcaagaccatgttttagactcagtaactaaaaattctaccttaaaataaaacattgatccatcataacagaactagtggattcctaaagagacaaccaagtccaacactttctgaatatccaatatgcagaacactacgtgaagttttcaagggggagatgtgtcttgctgat

SEQ ID NO: 16 - DNA (genomic), 611 bp; single-stranded, linear
acagagtggtctcttacaccaaataagaactaattttttgtcagtgtgaagtaatgctgtgattttttttttaatgaatagttttgaaattaagactactgtttgaaaattagggttttgtttttttttctttcagcataccacttcataactgttcagtttgtacagtttgttccccctgttatacccagttgagcttgtttgtttcttcacagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtataaaggaaatgtttactgttttgaatttgcttcttcattcaaacatagaagtctaagtataaaattagtgttctttaggaggatatgactttcctctggatttctctggttgataatgttacttagccatgagaatgtttttcatagagttttt

SEQ ID NO: 17 - DNA (genomic), 448 bp; single-stranded, linear
gtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtacagtaagtaggtcatgtcacatttagaaatttcctgttaattttttttttaaactgggcatttgggcttttaaaacctgtgttctcacaaaaagcctataaaatgactctgtacatgcaactattcctttcaaactatcagaaatatttggaattacccttttaacttaaaagttaatgcttttgcagatatttgaaaactaacaatgaactttttcattcttaaatgattgtctctaggaaataaggtgaccctaaccctaatgattcgattcgactcga

SEQ ID NO: 18 - DNA (genomic), 500 bp; single-stranded, linear
ggttgtggttatacgagatcgtgctgttccactccaacctgggcaacaacagcgaaatctggctcaaaaaaaaaaaaaaagaaaaaagtggatttatttttattttacaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggtaaagtgttaccattttctcattcagtgtcattttaatctcttgtatgttatttttcagaaaactttcagtggaatcctttcatctcaaccagaactaagtcatttgtctacccccaaacctattactagcaaagggatatgtgattgccatgacaaatgagatcaatcattaatggctcatttgcttgggccaagtgcagggccacctattttaatca

SEQ ID NO: 19 - DNA (genomic), 748 bp; single-stranded, linear
aactattaacagccagtttatttttagagtactatggaaatgatggtgatttctaattaggatattgtaagagtaccatgtctatatatttccttttagtttgttaatgtgatggaatagttttcaaatatccttttttttttttttttaggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatggtaagttcagcatgcattatgtctgacttacagataaacacacacagacacacacacactcacatatccctgatcatttccatagtttgttacttcagttaaagatgtcaaattctatttcagatgcttttcttgtttggccgagaagacttaataaatgcataagtgaatttagtttcaaatgttgacaaattattaaagactaatgttaaggaatttcttttt

SEQ ID NO: 20 - DNA (genomic), 699 bp; single-stranded, linear
agaattatgaagagtttaaatttcttttatgtgcaatttatcattatttattaaatagccatgtttaaattgtagtactatgcactgttaataaacgagctattttttaatcaagaatcttcccaaatgtaatcagacttttaacagtttttatgttcatttagtcaccttaactaaatgtatgtgccaggcactgtcctgatagataaagtctttgcccctccaatagcttgcttttcacaattgtcctttgttttgttatagtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacaggtaatttaagttcattagcatgctgctgttttttttgtttgttttatcaggctctctccacttatttgatgccagatggctttattttataataataatgcagaatttcccagatctaaccttaattattaaatattatgtttgtttttacagttatctgtgtctttatgcctgattgcttctgaaataaagggttgtctcactgtgagaatatgggggatgtgcatgaaaaatgcacat

SEQ ID NO: 21 - DNA (genomic), 548 bp; single-stranded, linear
tggatccaattaaagtacttttgctttaattttacaaccttttatttatttcagaaataatgttaaacatgctgtttctaaacagtattggaaatgataataacaatggttgtcctccttaaattgtccttttagatattaagaaatttagtatagatgaaagcaattttaatctaggatccaaattttagaagtcaagatttatagctaaacatggatgttaaagtttaaagtattctttacatggcttttggtcttctaagtgaagctttttgtttttctttgtagtaattttcctcatcttgtactggagaaaattcttgtgagtctcactatgaaaaactgtaaagctgcaatgaattttttccaaagcgtgccagaatggtatgttatctaataatgctctttatcattttaagctatagctttaattacaaagatgataattttcgctgggtagtagctgcatcttaataaggtcacctaacttggtccaaaaaaattgcaactgttagccagggaagaggttgttttaattcagtgattgt

SEQ ID NO: 22 - DNA (genomic), 637 bp; single-stranded, linear
tttctactgaataatgacatttgatataagtaggtctcaaagtccgaagaagagaaggcatttaaaagaataatctattaattatatgaagtagtctttgaatgatgtagatactaggttaatgttttcctttgtaatatattgctaatacatataaggcaaagcattaggtacttggtttatatattaaagatcttactttcttgaagtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgaggtgagattttttaaaaaaagaactaagcttatatatgattcaactttggtaaactgttaggaaggagaaataggggcaggaaaaacagcaaggatggtgggaggcttcattttaaaagcaaagtggcagtaaagggctctaaattggacaacttagcataattaaaggaaaactcaagaataataatttgagtacttccttt

SEQ ID NO: 23 - DNA (genomic), 568 bp; single-stranded, linear
ccatcaggagatacttaggctatttttcttgagaatcctggttataattctacagtgatctcctagttgtttttagagctatccaggatatgccacctttaactcagttaactgaacttttgttttttaatatgtatgtagaatttgttcttacaaaagatagagtatactaaattatttatgaaatatatatatttttatttgtggtttactttaagattacaaattcagaaactcttgtccggtgttcacgtcttttggtgggtgtccttggctgctactgttacatgggtgtaatagctgaagaggaagcatataagtcagaattattccagaaagccaaggtaggagaatttatactaataaagtttcggataaatttgaatgaaatgtattcctgtgaaaattattacatttgtttggaagacattaaattgtatgcaggttaaccctttctcttttatttatgtaatgtgagaagaaattatactatgtattttttaaattgttttaattgtttaatttttaattattattatactttaagttctggggtacatgatgcaga

SEQ ID NO: 24 - DNA (genomic), 531 bp; single-stranded, linear
aagatttgccattttaaaaaattgttaatgagttttgcttaaactgtatgaccacgtggaacttctaaaaacatttcattttttctcttaagtgcactttattttttattttatagtatgtccaagatcaaagtacactgtaaaaagcaatactaaactataattttaactggaatttgcatttttccttctattcacaatagtctctaatgcaatgtgcaggagaaagtatcactctgtttaaaaataagacaaatgaggaattcagaattggttccttgagaaatatgatgcagctatgtacacgttgcttgagcaactgtaccaaggtaagattttcttcttcttgttttgttttttgagataggatctttctctgtcacccaggctggagtgcagtgggattgtcacaactcattgtagccttgacctcctggtttccagcaattctcctgcctcagtctcccaagtaattgggactacaggcatgtaccacctagctaaaattttctttttacttgaaagtgtagc

SEQ ID NO: 25 - DNA (genomic), 476 bp; single-stranded, linear
tgttgttaaagcacaatgaaagatgtacagtatagttattataactctaagaaaagatgtgtttttgaagcagcatatatattggccctaatagtaaactatttatctacattccattcaagatagagaaaacactgtctgccaagaataattgtttttatttctttgttgcttggttctttgtttgtcttaattgcagaagagtccaaataagattgcatctggctttttcctgcgattgttaacatcaaagctaatgaatgacattgcagatatttgtaaaagtttagtaagtatgcttcctgttttgctatcatattttgattctaataggcataatttttttgttgaaatatctttgtaaataaggatgcatctcacaacatatagctcttaacatttttacaaatgtggaaattaaggccaggtgcggtggctcatgcctgtaaccccagcactttgggaggccgaggtgg

SEQ ID NO: 26 - DNA (genomic), 634 bp; single-stranded, linear
ttacaggtgcccaccaccacacccagctaatttttgtattttaaatagagacaaggtttcaccatgttggccaggctggtttcgaactcccgacctcaggtgatccacctggctcagcctcccaaattgctgagattacagatgtgagccactgtgcccagcctgattaggtaaattttgactacagcatgctcctgcaagaagccatcttgaacatctttgtttctcttccttgaaggcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtaaatacatatttactacttgggatttcttttacttctttatattgatttggcagtataagaggcctcattgatatcaattttgtgcttatttcattttctcttagtatagccttttaggattgttcctttcttatatactttattttttttttatttttactggaatttattagtttcatattttatcctccatagaaggaacttaagataactattaaaga

SEQ ID NO: 27 - DNA (genomic), 593 bp; single-stranded, linear
tttaaattatttcttgacaacagaatcttggataatttttcaaaaagacttttgaagctttcagtatataattaatttcactataattttgcttttcatatacttttttttgtgaagaggaggaaatttgagttaatatgactatatatggctgttgtgcccttctcttagtgttaatgagtgctttttatttttaggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatggtgagttacgttaaatgaagaagctcttggattttatctgatgttgctgactaaatgtaatgagttgacatgtaagaatcacatggtgtctttgaagaattgaaattgctttcttgagaaatgaacctgagactagttggaaaataacacttttaacgtgctgtgagcaaatttaagtggatgctgaaatattaaa

SEQ ID NO: 28 - DNA (genomic), 769 bp; single-stranded, linear
aggcgcctgccaccacccctggctaatttttgtgtttttagtagagatggagtttcaccatattggccaggctgttctcaaactcctgaccttgtgatctgcctgcttcagcctcccaaagtgctgggattacaggtttgagccactgcacccggcctatgtttatatactttttaaagtaaatgatttgtggataaacctgatttttttccctcctaccatcttagtatctaatgcttttaaaggagcttcctggagaagagtaccccttgccaatggaagatgttcttgaacttctgaaaccactatcgtaagaaattaaaacccttatgttatgttcactttaaagttataaaataactgatgtgttcttaagcttaataaagtggaactttttttttttttttaccacagcaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggtaggtacagtctattttgtggtcctatttttcttttgctatctgtggatacgaatgcaagttttgtatccacatcagtgatttcttctgatctgcctacatagctaatacatctggaaagaatagcagaatgttatttgtgtttccctcagtcgcttgaagaactacattgctttttgtttaaggcttggctttctaa

SEQ ID NO: 29 - DNA (genomic), 712 bp; single-stranded, linear
attccttttaccactaatttccttttagcttgaatttttggcaaggtgagtatgttggcatattccacataatgacaaataagtttagcacagaaagacatattggaagtaacttataataacctttcagtgagttttctgagtgcttttatcagaatgattatttaactttggaaaacttacttgatttcaggcatctaacaaaggagaggaaatatatattctctgtaagaatggccctagtaaattgccttaaaactttgcttgaggtgagtttttgcatttttttagtaagatctccattgaaaattttaaagcagtctttgtttgttaatgagtaatttttctctatttcatatttaaccacagttcttttcccgtaggctgatccttattcaaaatgggccattcttaatgtaatgggaaaagactttcctgtaaatgaagtatttacacaatttcttgctgacaatcatcaccaagttcgcatgttggctgcagagtcaatcaataggtaatgggtcaaatattcatgaagtatttggaatgctgcagatggcagtagaatgtcttacatagtaacagctcacagttgcaatattaaaaatagctaacacttgttgagtatatacggtgtgcctggcatttatgtttattcttaattcttatacttctgtcacttagattctattatttccttcaatttataaat

SEQ ID NO: 30 - DNA (genomic), 508 bp; single-stranded, linear
ctaagctgctggtctgaacctctttaaataaactcaggttttgttagtctttaagaaagagctagtatgttattatgtctcacagagtgatttatttttgttctggaatatgctttggaaagtagggtttgaaattagaaaattatttcactttttgtttgtttgtttgcttgcttgttttaagattgttccaggacacgaagggagattcttccaggttactgaaagcacttcctttgaagcttcagcaaacagcttttgaaaatgcatacttgaaagctcaggaaggaatgagagaaatggtaattttaagtaacatgtatttgctgttatcatatgcttgctatgaatatcccataaattacttcaccaagtttggtataagagagtttataatccagtagtttacagtataaagctgctcttccccaactgtatgaattgattgaaactgcattctttctgggtcacaatgggtcaaatcatagcaatttcttttggtttagca

SEQ ID NO: 31 - DNA (genomic), 435 bp; single-stranded, linear
aaaattggtatgtaaacaattacaatttacattacaatttttttttaaatttctttttaagtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggtatatatggatgagtattttattagaagcttccttaggtcactgtgaaataatttaaaaagttaaagctagattttctgagtggcacttatttaagactaggaaacaattttattttttaggttgggaatattggaaagcagttatacaaaaactattcaaatggtatatttatggtatgcactgtttcttacattcca

SEQ ID NO: 32 - DNA (genomic), 548 bp; single-stranded, linear
tgctatgtgtcagatactgtgccagttgagtacattttcttaattattattcccatctcatagatgaggaaatcaagaaaagttgaatgaatgttgtttctaggtcctactctaaataatattaacaagcatttaaatgatttatttttttcatttttcttaacacattgactttttggttcgtgcaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctataggtaagtttatacatgacatatgtgaaatttgtttaatttaaaattagttaacaatacttagcaagtcccctcaccagcaacacacataccatacccatacacatgtgtgtgtgggagcctacatagtatgagaagcaggacagcttcttttaataagaatgtattgaagggagtcactggacttcagatc

SEQ ID NO: 33 - DNA (genomic), 631 bp; single-stranded, linear
atttttttaatgtgactatttagaatttacttaatttttccatttataaaattaaagaatgtttaataatctggataaagtatgatactttaatgctgatggtattaaaacagtttttaagaactattttataaaattttacttggaaaagttatatataacctgtattttaaatttttctatttttagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacaggtatggcttcaatttttatgtacttttcattccctgaatgatatgagatataacctttaagttttaaggctatttattcgatttattcgtatttatatattgaaacttagcttgtggtaatcattatctagcatagccaacccatgaatttttttggttatgtcgtgttgtctccctctgattggcttttaacta

SEQ ID NO: 34 - DNA (genomic), 499 bp; single-stranded, linear
gggggccttgtttggctgattttcatactttttcctctcagtctacaggttggctgcatagaagaaaaaggtagagttatttataatcttgtaaatcttggactttgagtcatctattttcttttacagtcatcgaatacttttggaaataaggtaatatatgccttttgagctgtcttgacgttcacagatataaaatattaaatatattttaattttgtgcccttgcagattgatcacttattcattagtaatttaccagagattgtggtggagttattgatgacgttacatgagccagcaaattctagtgccagtcagagcactgacctctgtgacttttcagggtatgtacattttaaacttagagaactagctctaacttcacaagtttttaaagaagtttattggttgacaccttcaatgtctatttcaatttatagacatcactctttttaaaaaattttcttcaaaaatagccacctttgaattgaggtaa

SEQ ID NO: 35 - DNA (genomic), 1215 bp; single-stranded, linear
taatctgatttatatatctggactgtgatatgtcatttgtgattttattgaaagtatagtttttcagtagaaaaatggtttttgaatttgggggttattaaaatctaaattttcattttggaagttcactggctatgaacaaaactttttaaaacgatgactgtattttttcccttaactctgttagggatttggatcctgctcctaatccacctcattttccatcgcatgtgattaaagcaacatttgcctatatcagcaattgtcataaaaccaagttaaaaagcattttagaaattctttccaaaagccctgtaagtatacatgatgagtttaataatagaacattccttcttttttagctaaaaaaactttgtaaatacatcttaaagaggaaaagtaaacaaatgaaaaatttatctcataattaaaaaggaaaacattcatttacaagtttaaatggtattttacttgtcagcattaattgaaatatgttacatatgagaacagaatcttgtgacactttagtgatatattagctcagggaatatatctactttttcataggaatatactatttaattgtagtttactttctgaaaattaaataaattggcaatagtttaagatagtaattttcttaatgtaacattttgtacttgatatcaaacccaaatctaaattctgttatttagttattttaaatataaaatgtgtaggtattcaaatatttgaagaaaaaatataaagtgtatttattgtagccgagtatctaattaaacaagtttttactaaatctgtttattttcttaggattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggtaaataacatatttagaccaatatataagcagtctttctatcctgttcttcctgtttttttgctttgttttgttttgttttgagacaaagactcactctgtccgcccaggctgggtgcagtcacggctcactgcatcctcaacctcctgggttcagattatccttcctcctcaccctgccgggtagctggggctacaggag

SEQ ID NO: 36 - DNA (genomic), 1235 bp; single-stranded, linear
atgtttctattaaaggatggaagcttagagctgcctattctgcattttgctgatgtgacttctctttttggcttataagccattaaaatattttgtcaaggcatataagaattagagatgctgaaccaaaggacttctgaatgaatttatttcagagtaattttccagaacttactggttgttgttgtttttttttctccctatattaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtaattttctgactcatcttcaaaaatggtatttaaaatatataaagtattgttagaaggatttgagtgttttatgtttatttggtataattggtgattttattgagaatattttttgtaaaatgattggaaaaatattcttaatgaattaacctttgtaatcaattacagagcacttggtacttttgatagttttatctactgtgctgaagtggagaggtagtcaaaactagggatagcagttcgcaacgttatggtggtatttgagttactacttatataaactgtttcattaatattggcattttttttaacctcagtacccatcttgtagtagtaccttacatagttattgaattatttgaaaacacagaaactaaaagctgggtatcttagacgtaataagaacatttaatctgatctaggttaatagattttatcatttattacagtaagttttgttggcttactttaaaattatttctctccttataattttttctttttaaattatatttaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggtaataaaaatttcatcatctactattttttattagagaacatagtagtacttttcaaaaatctgtaatgctctagcagtaaaaaatggaatcttttctttaattgtgattaaaaatatatacgtaggccaggcacattggctcatgcctataatctcagcacttt

SEQ ID NO: 37 - DNA (genomic), 541 bp; single-stranded, linear
gactctgtctaaaaaagagtattaaaacattgtagggtttgcagtggaagaaatcatttatttcttccttgattagtagtaatagagacatgagtcagtgtctataaatggcacttaactaatttttttcttttattaagttttatttcacaggcttaaccaatacgtgttaaaagcaagttacattttctcttttaggaaattaaccattttctctcagtaagtgtttatgatgcacttccattgacaagacttgaaggactaaaggatcttcgaagacaactggaactacataaagatcagatggtggacattatgagagcttctcagggtgctaattttaaatgacatgggctatttctacctgtttctttttggaaagaatattttgcaaagtcttgctcttggtttcattgtcacagacttagttcagactctcatcatttagttcagaccctcatttctcatctaactgtaaaactggtcctaactggtcttctcaccctgaactcttcctgttttattcatcctctgccagatg

SEQ ID NO: 38 - DNA (genomic), 537 bp; single-stranded, linear
acctctgtctcccaaagtgctgggattacagtcgtgagccaccgcactcggccttaaggttaattcttgaagtacagaaaaacagcattatagtttggaaattagaaaatatcagttttatgtatgatctcttacctatgactctactgaaatagaatttctatatgtagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattggtgagtatttattgataccttatatgtaatctcaatatgacattcatggagaatgatacttcacacaaatagatattctcagtaactaaagctttgtccttttttaaatctcagtgtctttatgaaaattcttatatttttattaattcacataattatttaccctactatgtgccagacacttgatataatggt

SEQ ID NO: 39 - DNA (genomic), 531 bp; single-stranded, linear
gttgcagtgattagtaattcaagtttactgaatgactagtgaaagtcctttgatacttttatttgatattggagaattttgtaaatgtaaagtttcctaaaaccaattttaaattttagttttgaaattttttcagtggaggttaacattcatcaagattaataactggtgtacttgataggcatttgaattgtttttttcagtgtcaaagttcgatcagcagctgttacctgtttgaaaaacattttagccacaaagactggacatagtttctgggagatttataagatgacaacagatccaatgctggcctatctacagccttttagaacatcaagaaaaaaggtctcttaagtaataaatgtttattgaatacccagcatatctaaaacagttctgtttgctgtgggtcatgactgttaaattgcttgaaatagtattgtactaactattaacctttcctataagtaatttaagccatatttcataaatccagggaatgtgttatttttaatttattatggcagtgtg

SEQ ID NO: 40 - DNA (genomic), 527 bp; single-stranded, linear
aaacataaatgttttcatcttaaaaggtaaacattgcctccagatttagttttaactgtatttagctttattcagaaagatttgttatactcattttgtgtaggaaaggtacaatgatttccacttctcttatttacattttctaatccctttctttctagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtaagaagattaattagtctgatataattccttgtttatgacctgtttatctaaagagtgctgtgatactgcacatcatcttcacataatatcacccccactcaaactgttgtaaatttattaaagtgagcatccgtatttagtcataactttatgcattaggtttcagcttcgggatagcaacatact

SEQ ID NO: 41 - DNA (genomic), 567 bp; single-stranded, linear
cctagtgtggttttttaaacaccacctaatacatgttttttgtttgtttttttagcagtatgttgagtttatggcagattaatctatcatcttttagaaatttaatatgtcaacggggcatgaaaattttaagtaaaatgtattaattttactcatttttactcaaactattgggtggatttgtttgtatattctaggtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcaggtattctattaaatttttaacattaatactgtaaactcagttctagagaaagatggatttaagatggaatcccactaaaagcactttacaggattaaatctataacctctaaatttgtttcttcatctatggaatggagataaaagttgccaacagttgcaacaagttttcaatgaaataatgtgtgtaaag

SEQ ID NO: 42 - DNA (genomic), 475 bp; single-stranded, linear
taagttctcactttttttaagataacagtttcttttaaaagcaaaagaaatcctattaaattccttcagaaccaattttgtgttaggtactgcccaccagaaccttatagcatagtgggagacagacacataaacaagaaggaagaaggtgtgtaagcaagaatgcctgggactgaggggagatatttttgtttgtcagagtcagagcactttttccgatgctgtttggataaaaaatcacaaagaacaatgcttgctgttgtggactacatgagaagacaaaagaggtaatgtaatgagtgttgcttcttacgtttaggatctagagtgtaacttgttaactatcggctgaattttaacatgattattttaggtgaaggtgttgcaaagtgttatatttaatttgtgtgatatttatatctccttgcagtaatccatattcaggatagcagtttggttaaatcagtgtcaagaa

SEQ ID NO: 43 - DNA (genomic), 528 bp; single-stranded, linear
ttaattaaataggactcttcagccatgttatcttataatgtttataggtatatattggggaaatgtggtttttgggaatttgtaattttctgttaagcagtcactaccattgtattctatatcaacatgcttttattttgatattgaagtttaaaaaagtgaatgacattatatctcatttttctttagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaaggtaatggaatttagaatttttggtttttaaaattaatgttggcattgtctcaataagggtatatagtaaagatttattttgcctcctgttccccatttaaaagatattttagatagaaattttgttttaaagtgaaattataataaatttttaaaaaggaatatgtaattcctgttctgaaat

SEQ ID NO: 44 - DNA (genomic), 444 bp; single-stranded, linear
cttttccatcctaggtataaatggtattatgttttaaagtataagtgatttattctgttttgtttgccaccttcattagtttttttctgtcaaagtctatagtatatgtattcaggagcttccaaatagtatgttctcattaaaagaggtgttcttgtgacaaacagaagtcttgcatttgaagaaggaagccagagtacaactatttctagcttgagtgaaaaaagtaaagaagaaactggaataagtttacaggtaaatattagaggctctattatttatgacagtatttatctcatactttgggttattttgttatagacactgtacagatgccatgtgatttttaaactgaatttacttactggactaagcatcatatatataaaattatggtctgaagcttaagccttagagtagacagacttgagttctaatactgac

SEQ ID NO: 45 - DNA (genomic), 688 bp; single-stranded, linear
ttgtcctgcacagttcaaactcgtgttgtttgaactgtatttcagaactgtatttcagaatcattacattttatttctataacataacatttagagttgggagttacatattggtaatgatacaatttaaaatttgctaaatttatagaccgattttttttccttcttcaatttttgttgtttccatgttttcaggatcttctcttagaaatctacagaagtataggggagccagatagtttgtatggctgtggtggagggaagatgttacaacccattactaggtaaattgcatttttctaaacaacggtatagtaattctgtttatgaaggagttatgtgtgtgttaaacccaaagctattttcacaatcttttcttatagactacgaacatatgaacacgaagcaatgtggggcaaagccctagtaacatatgacctcgaaacagcaatcccctcatcaacacgccaggcaggaatcattcaggtacattttttcccagatttggtaaagccatcactagtgtagtgctgaggttatttcagtatgttggtggatatttacacagccagataaactctagagataagactagaacttatctgtttttcagaggattaggctaaacattcagggatactcctgaagcagagggatgcaaaaaaaagagaaaaaattcagggagaca

SEQ ID NO: 46 - DNA (genomic), 418 bp; single-stranded, linear
cctcctgcctcagcctcccaaagtgctgagattacaggcatgagccaccacacccagctgatattttgggattttaaatgatattgtgaactaaaatttgtctaagttaatttgtatctttgctgtttttttctctggttttctgttgatatctttgattacttaacttaaaaacaaaataactcctgtttaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcaggtaagaaatttgacttgatttttttttttttgcctctctcctcattctaaacaacaactgtttttctcttctatga

SEQ ID NO: 47 - DNA (genomic), 513 bp; single-stranded, linear
tttttctgcttaaagaatttaaatgactcataaaatttgtatttcttaccaaaaattctagaaatgcattttttagaatggagaaatgttaatttaaaaattttgtcctttggtgaagctatttatacatgtatatcttagggttctgtttttaagtatatttttttctttgacttatctcacagcaaagaagtagaaggaaccagttaccatgaatcattgtacaatgctctacaatctctaagagacagagaattctctacattttatgaaagtctcaaatatgccaggtattatgaaaagacaaagttactgtattttaacatttaatgtcatggcttcttttctgaaaacttgagaaacaattttaatgtaaggatttgcattgatgaagagataaagacttggtggctgtgatcagatgtttccttgtaattctctgccctccttcaaaacaaattgtttctgggattccaggttcattctttaccctgacccttcaagaaagttttg

SEQ ID NO: 48 - DNA (genomic), 513 bp; single-stranded, linear
agtgtaagtggaccatgcatttaaatttgtgttgttcaagggtcagttgtattctgtttccactgctattttgtactcactgctgcttgttagtattattagatcagtagcatagcctatgatgagaactctttaacaacaaatttaaacatttatttccctgcaaacctcttctttattttcagagtgtcttttcttttttgctactagagtaaaagaagtggaagagatgtgtaagcgcagccttgagtctgtgtattcgctctatcccacacttagcaggttgcaggccattggagagctggaaagcattggggagcttttctcaaggtatgtaattcgtatgactttgttatcctaaagtgcagcttttctgttaccaatagtgactttaaaaaataaaaactataggccgggcacggtggctcatgcctgtaatcctagcactttagaaggctgaagtgggtggatcacttgaggtcaggagttcaagaccagcctggccaacatggt

SEQ ID NO: 49 - DNA (genomic), 1254 bp; single-stranded, linear
tgtattattataatattatatcgtaagttccaggacttacatagttttttttttttttttttcatttctcttgcttacatgaactctatgtcgtggcattcagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcaggtaaatacaatttaaaactatgtcatcttacctcttgactttccttttattatttaaaaaactgaaagcctgagggaaaaagaaatgtcattaagagatagagatctctattaatatatagtaaaaataattgtttaagagttcccattttggaattagatctgacttttaagccttgggcaagggtacttaatcttttctcaacctcaatttcctggttataaaatgagaagatacctaacttactatattgataacaattcagtgattttatatactgtgtgtatgtacacacagatacacatacatacatatagagagagacagacagacagacagataggcagacgtggggtggggagattgtcaatgcagacagagagggtccttaaagatagtccctgacaagtagttaaagtcctcaaatgaatggtagttgctgctttcattattattattattcaaggtagtagtatcaagtagtaaaagtatttattcccatatgtcattttcatttcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcggtttgttttttttattggctggattagtgttttactgttatttaaaaaaacacaaatgtactttaaaatatttttaataacaattttattagagccttgaaattagtaatttattaacaagatattgtaaaactagtcttgaaaattaatttgtaaatgaagtttagaaactttttcctatatatcacaattctatcagtccatcatgtggtcgattcatttaatata

SEQ ID NO: 50 - DNA (genomic), 537 bp; single-stranded, linear
taataataataaacagaggatgatcatttcctacatgggattattaaaatagttgtatggcaaaagcagatgaggaaaaactttttttttcccacccaccaaggaaaaacatttttaacctgcttttttcccccgtacatgaagggcagttgggtacagtcatggtaatgcattatattttaagattttgcctttcttatacagaacaatcccagcctaaaacttacatacacagaatgtctgagggtttgtggcaactggttagcagaaacgtgcttagaaaatcctgcggtcatcatgcagacctatctagaaaaggtaagatttttggagcaacccttaagatagttacttagcatgaatatgcttcatcttttcatcaagatcaatatatttccaaagcaaataaaagtatggttttatttttctatatattattactgttgtagctctgtatagtctctagggtggagtgaaacattgttacaaaacaaagcagccaatttgaaaagtaagcccaagtatagtatctcttct

SEQ ID NO: 51 - DNA (genomic), 679 bp; single-stranded, linear
tttaagaaaatgtacgaatttgtgttgggccacattcaaagccgtcctgggccacatgcggcccatgggccgtgggttggacaagtttgcaatagttcatataatttagctagcttttatatgtatataagttaaattttagtgtattaccttaatttgagtgattctttagatgtatttagtatttgtaaatataatttaaattggttgtgttttcttgaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacaggtaactaggtttctacaagtgacaattttatgttcaccagttaactgagtgagtgtttttgcatagaaagagtgacttggtctttttatctgatatagttttgagctctaaaggtcggcttaactatatatagattatcttggtcttttgggttcttttcggtttttgttttttgtgtttttttttgagacaaggtctcactctgtcacccaggctggagtacagtggcgtgatcact

SEQ ID NO: 52 - DNA (genomic), 610 bp; single-stranded, linear
tcccctttgtcctttgatgcttaggaaggtgtgtgaattgcacagttaagacaaaagtaagtttattccctttataatccttagaagtttgcttttttccctgggataaaaacccaacttttttcattaaatgttgtatatcatgtgtgattttgtagttctgttaaagttcatggcttttgtgttttaccttaattattctatgcaagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccggctttgttccctctggcttgaaaattctggagtttctgaagtcaatggcatgatgaaggcaagtgttactcagcccaatattctaccctgtgcttgaaaaacttagacataagccccttgatgtcaggaatcgtgtatacctctttgtattcctagcacttggtccagtgctctacacataagtagcattttgtagttttctaaactttgatccatatttaggattatttacaagttctagtcttgtttct

SEQ ID NO: 53 - DNA (genomic), 988 bp; single-stranded, linear
aatccagtttaatttaggacccaatattttgatttaccaatgcattaatctagagtacccattagaaagaccttcagataagaaaagaaatgaaggaaaacaatatagttagtgaagttttgttaaccacttgtgctaatagaggagcactgtcttaaaataacttacttgcttagatgtgagaatatttgaaataccttgtttcttaattttgtgtcttttttttaatggtagagagacggaatgaagattccaacatataaatttttgcctcttatgtaccaattggctgctagaatggggaccaagatgatgggaggcctaggatttcatgaagtcctcaataatgtaagtaaacctgaaaatcaaaccacaataattatttttattctattattactatatattatataaagtatatataccattccctctaagaaatggaaatacaaaattttgtattttttgtcttctcacatcacataagttactcattttctctctctaattcctcataggcctctgcctttttctcacacatgcaggcatacacgctctacccactgcagtatctagacagtaatacacattttaatgttaagcaaaatgaaaaatatggattatatttttttgtttatttgcataaatctaatagttcttttcttacagctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggtattgggattaaccatacgtaccttttagaagtgtgatattcagtctttcctagaatatttctttttaaaatcttgtgttattaagatgccatctaaaatcggttcaaggctggcacggtggctcacgcctgtaatcccagcactttgggaggctgagg

SEQ ID NO: 54 - DNA (genomic), 537 bp; single-stranded, linear
gaaaggcacctaagtcattgacgagagtatgtatctttgatgtatttcatttatgactgttttgtttgtatctgaggaattataatcattccatagtctagatttgtgcataaattctgtttttctctttgtttttctaactctgagaagtttaaatgttgggtagttccttatgtaatgttttttgttttttattaataggatcgaacagaggctgcaaatagaataatatgtactatcagaagtaggagacctcagatggtcagaagtgttgaggcactttgtgatgcttatattatattagcaaacttagatgccactcagtggaagactcagagaagtatgttttttttaaagaagaaacgttactttcttgctgtgttactctctgtagagatatattagttatagagcctaataagtaaatctgcttaaaatcacaaacgtaatccaaaagcttaatttatatctgatggcttcaacattccctggttactttttcacttaatatctcttaatagaactggtaataggtga

SEQ ID NO: 55 - DNA (genomic), 421 bp; single-stranded, linear
ctgttgagctttgactctgagctgcatagtggccaaagcccagagtcttcatttctcaatcagagcctgaaccacagattagcaacaagttggggccagtggtatctgctgactattcctgcttgaccttcaatgctgttcctcagtttgtcactaaaatctcttcatttttaaatacagaaggcataaatattccagcagaccagccaattactaaacttaagaatttagaagatgttgttgtccctactatggaaattaaggtaatttgcaattaactcttgattttttttaaactaaattttttttattagattgaaccatttgaaatagtatttttatgtaggtcaaaattggttaaatattggcaaatttcatatgtttcaacctataatttctcagtattatatttcctttgccc

SEQ ID NO: 56 - DNA (genomic), 449 bp; single-stranded, linear
atattagtgaatctttgatgaaacagtagttaaagttacgagcgtgagccaccacacccggcctaaagttgtagttcttaaccactatcacatcgtcatttgtttctctgtttaatattaaaattgccatttataatgtattgtgctttaagtgcaaatagtgtatccgacctattagcaatcatgtttatacttttattaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaaggtgagccttcccttctctggcttagcccttagagttttagtgatgaaaatttttagttcatatttctttctgctttattggggatttgggtctttatttgggaata

SEQ ID NO: 57 - DNA (genomic), 520 bp; single-stranded, linear
gggaaactttctaaatcagtgtaaatgttgtagcttattctaaatgaaagaatggcagtaggtatttaattatttgggagactgtcaagaggtgcacagatgctcagattggtttgagtgccctttgctattctcagatgactctgtgtttttataataaaataaactgtacttgtttattcatgcttaattattctgaagggccgtgatgacctgagacaagatgctgtcatgcaacaggtcttccagatgtgtaatacattactgcagagaaacacggaaactaggaagaggaaattaactatctgtacttataaggtaactatttgtacttctgttagttcaccaaaaacatataaaagatgccatttggttgggtgaagtggctcatgcccatattcataatgctttgggaggccaaggtgggaggattgcttgaggccaggagttcgagaccagcctcagcaacatagtgagaccccatcttgacaaaaagttaaaaaaaaaaaaaaaaccagag

SEQ ID NO: 58 - DNA (genomic), 519 bp; single-stranded, linear
ttctttgagcttaagtttatttccgattggtttcctccaaggagctttgtcttctatggacagagaaatattaatacaacttgaaaaaaaatgctttgcactgactctgatagctgaatgatcatcaaatgctctttaatggccttttaaaagtaaaaggtatttaatctgtaactccaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggtgagtgacacccaaaattaaaggttattgtaagattatttaatggcttattaaagctgacagctgtcagatattatagaatacaaaaaaactttaatttcatcaggtaattgtcaaagatactaagtaaaagaaaaactcatcagaatgaaagtgtgtgagtgaaaaaggaaggattttaaactacat

SEQ ID NO: 59 - DNA (genomic), 550 bp; single-stranded, linear
atcacttgtaattaattgcttccctgtccagactgttagcttcttgtaggtaatgtatcctgttcatctttattgcccctatatctgtcatatttttatataaaaatgtgtatattagtttaattgaacacaatattgaaaaataattatatatattctctatttaaaggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattggtaatcttcttgtacatatagtagattgagcactttgttgtttggcaggttttatttttgtttgattcagcactttttctacattctgagttgcagggggatgatagtgatgatgtggttagtaaccatcccatcttcattattaaatcatatgtttcttgttcatcctgattcttagtgtctacctttttataacttatgcagaagagaattct

SEQ ID NO: 60 - DNA (genomic), 474 bp; single-stranded, linear
aatcatctaggatttgtaaaatgcaatatgcattaaaatagctggcaagatttgagttaaactcaacatggccggttatgcacatcatttaagtaggctaaaaatcctaaactacttaaagattataccaagtcagtggtcttaattgaaattatggctatatattagaaagagatggaatcagtgatttcagattgtttgtttcttttttctccagttggttacatacttggacttggtgatagacatgtacagaatatcttgataaatgagcagtcagcagaacttgtacatatagatctaggtaagtaataaaatctatgtatctattctttttagtaaatatttggtcatcatggaatgttgtttgcctaccaagatattacaaatataagagacagataaatcgaagcagtaaatattgggtttttttgttttcagcataaacagttgtcctagaagaaacagttaact

SEQ ID NO: 61 - DNA (genomic), 483 bp; single-stranded, linear
gatttgaggtggatctcacagacagtgacaaagatgaggaaggcagccagagcagaagtaaactactgtacatactagtgttcatagaacgtaggtaacatgtggtttcttgcctttgtaaagttcacattctaactggaaagaaagtaaattagctgtcaaacctcctaacttcactgtattctttactttaggtgttgcttttgaacagggcaaaatccttcctactcctgagacagttccttttagactcaccagagatattgtggatggcatgggcattacgggtgttgaaggtgtcttcagaaggtaagtgatatgaagtaaaggagggaaataatttttgatgtcaaaattacatgggctgggcatggttctttgcacctgtaatcccagctactcaagaggctgaagtgggaggattgtttgagcccaggagtttgagtccagcctaggcaatacagcaagaccctgtatctaaaa

SEQ ID NO: 62 - DNA (genomic), 318 bp; single-stranded, linear
ttcttgtaaatgccaagcttgtgaaatagtcaaatacatatttgtattcatttcaaacgtctaatgaaagcccactctgccaagtattatgctattttgagatacagatatgtagattattaagcataggctcagcatactacacatgagagtatacagataaagatatgttgacaacattggtgtgtaacaaaatccgtatttataatgtgtttgactctagatgctgtgagaaaaccatggaagtgatgagaaactctcaggaaactctgttaaccattgtagaggtaaagtattttataaggaagactttatttt

SEQ ID NO: 63 - DNA (genomic), 453 bp; single-stranded, linear
aagaacagatgttctctctgtttaggtccttctatatgatccactctttgactggaccatgaatcctttgaaagctttgtatttacagcagaggccggaagatgaaactgagcttcaccctactctgaatgcagatgaccaagaatgcaaacgaaatctcaggtgagcagtattttaagaaggtcctgttgtcagtatttcagattttcttattcccaaggcctttaaactggtcacctggactggaacctttgtgtttttgtccttagtgatattgaccagagtttcgacaaagtagctgaacgtgtcttaatgagactacaagagaaactgaaaggagtggaagaaggcactgtgctcagtgttggtggacaggtgaatttgctcatacagcaggccatagaccccaaaaatctcagccgacttttcccaggatggaaagcttgggtgtga

SEQ ID NO: 64 - DNA (other nucleic acid), 21 bp; single-stranded, linear
  description: /desc = “primer”
gtgcagtgaggcatacatcac

SEQ ID NO: 65 - DNA (other nucleic acid), 23 bp; single-stranded, linear
  description: /desc = “primer”
aaggctgaatgaaagggtaattc

SEQ ID NO: 66 - DNA, 20 bp; single-stranded, linear
caccctgctgcccagatatg

SEQ ID NO: 67 - DNA, 24 bp; single-stranded, linear
cttataccacgaaaggtaatacac

SEQ ID NO: 68 - DNA, 23 bp; single-stranded, linear
gaggtcaaacctagaaagctcac

SEQ ID NO: 69 - DNA, 20 bp; single-stranded, linear
cctctcctttgttagatgcc

SEQ ID NO: 70 - DNA, 23 bp; single-stranded, linear
ctaggtcaaagcaatatggactc

SEQ ID NO: 71 - DNA, 21 bp; single-stranded, linear
catgcgatggaaaatgaggtg

SEQ ID NO: 72 - DNA, 23 bp; single-stranded, linear
cagagattgtggtggagttattg

SEQ ID NO: 73 - DNA, 22 bp; single-stranded, linear
gcattatgaaggtccactgaag

SEQ ID NO: 74 - DNA, 22 bp; single-stranded, linear
cttcagtggaccttcataatgc

SEQ ID NO: 75 - DNA, 21 bp; single-stranded, linear
ccatacaaactatctggctcc

SEQ ID NO: 76 - DNA, 24 bp; single-stranded, linear
ctggaataagtttacaggatcttc

SEQ ID NO: 77 - DNA, 25 bp; single-stranded, linear
gatgatttcatgtagttttcaattc

SEQ ID NO: 78 - DNA, 22 bp; single-stranded, linear
gatggagaaagtagtgatgagc

SEQ ID NO: 79 - DNA, 23 bp; single-stranded, linear
agtcaccagatttccatattctc

SEQ ID NO: 80 - DNA, 23 bp; single-stranded, linear
aagatgttgttgtccctactatg

SEQ ID NO: 81 - DNA, 23 bp; single-stranded, linear
aaggctgaatgaaagggtaattc

SEQ ID NO: 82 - DNA (other nucleic acid), 24 bp; single-stranded, linear
  description: /desc = “5′ primer for exon 4-5”
cacacctctttctctctatatatg

SEQ ID NO: 83 - DNA (other nucleic acid), 24 bp; single-stranded, linear
  description: /desc = “3′ primer for exon 4-5”
cacacaaaagtaatatcacaacag

SEQ ID NO: 84 - DNA (other nucleic acid), 23 bp; single-stranded, linear
  description: /desc = “5′ primer for exon 17”
gtatgtccaagatcaaagtacac

SEQ ID NO: 85 - DNA (other nucleic acid), 24 bp; single-stranded, linear
  description: /desc = “3′ primer for exon 17”
ggtgacagagaaagatcctatctc

SEQ ID NO: 86 - DNA (other nucleic acid), 24 bp; single-stranded, linear
  description: /desc = “5′ primer for exon 25”
ctggaatatgctttggaaagtagg

SEQ ID NO: 87 - DNA (other nucleic acid), 25 bp; single-stranded, linear
  description: /desc = “3′ primer for exon 25”
ccaaacttggtgaagtaatttatgg

SEQ ID NO: 88 - DNA (other nucleic acid), 21 bp; single-stranded, linear
  description: /desc = “5′ primer for exon 34”
cacaggcttaaccaatacgtg

SEQ ID NO: 89 - DNA (other nucleic acid), 21 bp; single-stranded, linear
  description: /desc = “3′ primer for exon 34”
caggtagaaatagcccatgtc

SEQ ID NO: 90 - DNA (other nucleic acid), 24 bp; single-stranded, linear
  description: /desc = “5′ primer for exon 46”
gtcctttggtgaagctatttatac

SEQ ID NO: 91 - DNA (other nucleic acid), 26 bp; single-stranded, linear
  description: /desc = “3′ primer for exon 46”
ccaagtctttatctcttcatcaatgc